SEQUENCE LISTING (51 sequences)

SEQ ID NO: 1 - DNA, 4476 bp, Artificial Sequence
  A synthetic oligonucleotide sequence
ctaaattcggccttgctcagagactcctggattttggctaacaacgcagtcccttcgatgcatatagctaggccacaaattatgccaataacggtccatgggttgatgttttcttgaattctttcgtttttcatgctatttgcgtcttcccaagtcccagcgttccagtattcatactgcgcgttagagtggtagccataagagccggcatattggtaattttcagtattaacgttagaacgtggtgaatacgatgtggtccagccttgcctcgttgtgtcatatacgatctttttctttgggtcacaaagaatatcatatgcttgagagatgactttaaatctatgtagtttttcgcttgatgttagcagcagcggtgatttactatcactgttggtaaccttttctgagctaaatatttgaatgttatcggaatggtcagggtggtacaattttacataacgatgatattttttttttaacgacttcttgtccagtttaggatttccagatccggcctttggaatgccaaaaatatcatagggagttggatctgccaactcaggccattgttcatcccttatcgtaagttttctattgccatttttatcgttcgctgtagcatacttagctataaaagtgatttgtgggggacacttttctacacatgataagtgccacttgaataaaaatgggtatacgaacttatggtgtagcataacaaatatattgcaagtagtgacctatggtgtgtagatatacgtacagttagttacgagcctaaagacacaacgtgtttgttaattatactgtcgctgtaatatcttctcttccattatcaccggtcattccttgcaggggcggtagtacccggagaccctgaacttttctttttttttttgcgaaattaaaaagttcattttcaattcgacaatgagatctacaagccattgttttatgttgatgagagccagcttaaagagttctcgagatctcccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagtttaaaacaccaagaacttagtttcgaataaacacacataaacaaacaaatctagaatgtctattagtgaaaaatattttcctcaagaacctcaatctcctgaattgaaaactgcaattccaggtcctcaatcaaaggcaaagctcgaggaattatctgctgtctatgatacaaaggctgcatattttgttaccgactactacaaatctcttggtaactatattgtggatgcagatggcaacaagctactagattcttattgccaaatctcttctatcgcattgggttacaataatccagcattattaaaagtagcacattctgatgaaatggcagttgctttatgtaacagacctgctttggcatgttttccatccactgattactatgaaatactaaagaagggattgttgtccgttgctccaaagggattagataaggtttgtactgcacacacgggatctgatgccaatgaaatggcatttaaggctgcatttttgtttcaagcaagtaagaagagaggtgacaaaccatttaccagcgaagagctggaatctgtcatggagaacaagttgccaggcacctctgacatggttatcctgtcatttgaaaaagggttccatggtagattgtttggatctttatctaccactagatctaaagctattcacaaactggatattcctgcgtttgaatggccaaaggctccattccctcagttaaagtatcctctggatcaattccaagctgaaaacaaagcagaagaagaaagatgtttgaaggctttagaggaaattattgtcaactctcctgccaaaattgcagctgcaatcattgaaccggtccaatctgaaggtggtgataatcatgcttcaccagaattcttccaaggtattagagaaatcaccaaaaagcacggtgtcattcttattgttgatgaagttcaaacaggaggtggtgcttctggtaagatgtggttacatgaacactatggcattgtcccagacatcatgactttttctaaaaaaatgcaaaatgcaggtttctttttcagtgaagcaggtcttgctggggaccaaccattcagacaattcaatacctggtgcggtgatccatcaaaagctctaattgcaagaaccataattgaagaaattaaagataagaacctattgactagtgttaccgaaacaggtgactacctatattcaaagctcgaagcaatttcagcaaagtatgacaaaatgatcaacttgagaggtaagggaagaggtttctttattgcatttgatgccccaacaccggagttaagaaacaaatttattgctgaatgtaagaaattaggtttaaacattggtggatgcggtgaacaaggtgttagattgagacctgcattagtttttgaaaagaagcatgctgatatcttagcctccattattgatcaagctttttccaaaatttaattaattaaacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgggcagacggccggccataacttcgtataatgtatgctatacgaagttatggcaacggttcatcatctcatggatctgcacatgaacaaacaccagagtcaaacgacgttgaaattgaggctactgcgccaattgatgacaatacagacgatgataacaaaccgaagttatctgatgtagaaaaggattagagatgctaagagatagtgatgatatttcataaataatgtaattctatatatgttaattaccttttttgcgaggcatatttatggtgaaggataagttttgaccatcaaagaaggttaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacgcggccgc

SEQ ID NO: 2 - DNA, 4355 bp, Artificial Sequence
  A synthetic oligonucleotide sequence
ggccgctccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaaagcttcatggcctttataaaaaggaaccatccaatacctcgccagaaccaagtaacagtattttacggggcacaaatcaagaacaataagacaggactgtaaagatggacgcattgaactccaaagaacaacaagagttccaaaaagtagtggaacaaaagcaaatgaaggatttcatgcgtttgataacttcgtataatgtatgctatacgaagttatctcgagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctgttgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaagtctagaatgtctattagtgaaaaatattttcctcaagaacctcaatctcctgaattgaaaactgcaattccaggtcctcaatcaaaggcaaagctcgaggaattatctgctgtctatgatacaaaggctgcatattttgttaccgactactacaaatctcttggtaactatattgtggatgcagatggcaacaagctactagattcttattgccaaatctcttctatcgcattgggttacaataatccagcattattaaaagtagcacattctgatgaaatggcagttgctttatgtaacagacctgctttggcatgttttccatccactgattactatgaaatactaaagaagggattgttgtccgttgctccaaagggattagataaggtttgtactgcacacacgggatctgatgccaatgaaatggcatttaaggctgcatttttgtttcaagcaagtaagaagagaggtgacaaaccatttaccagcgaagagctggaatctgtcatggagaacaagttgccaggcacctctgacatggttatcctgtcatttgaaaaagggttccatggtagattgtttggatctttatctaccactagatctaaagctattcacaaactggatattcctgcgtttgaatggccaaaggctccattccctcagttaaagtatcctctggatcaattccaagctgaaaacaaagcagaagaagaaagatgtttgaaggctttagaggaaattattgtcaactctcctgccaaaattgcagctgcaatcattgaaccggtccaatctgaaggtggtgataatcatgcttcaccagaattcttccaaggtattagagaaatcaccaaaaagcacggtgtcattcttattgttgatgaagttcaaacaggaggtggtgcttctggtaagatgtggttacatgaacactatggcattgtcccagacatcatgactttttctaaaaaaatgcaaaatgcaggtttctttttcagtgaagcaggtcttgctggggaccaaccattcagacaattcaatacctggtgcggtgatccatcaaaagctctaattgcaagaaccataattgaagaaattaaagataagaacctattgactagtgttaccgaaacaggtgactacctatattcaaagctcgaagcaatttcagcaaagtatgacaaaatgatcaacttgagaggtaagggaagaggtttctttattgcatttgatgccccaacaccggagttaagaaacaaatttattgctgaatgtaagaaattaggtttaaacattggtggatgcggtgaacaaggtgttagattgagacctgcattagtttttgaaaagaagcatgctgatatcttagcctccattattgatcaagctttttccaaaatttaattaattaatttaccagcttactatccttcttgaaaatatgcactctatatcttttagttcttaattgcaacacatagatttgctgtataacgaattttatgctatttttttaatttggagttcggtgatgaaagtgtcacagcgaatttcctcacatgtagggaccgaattgtttacaagttctctgtaccaccatggagacatcaaagattgaaaatctatggaaagatatggacggtagcaacaagaatatagcacgagccgcggagttcatttcgttacttttgatatcgctcacaactattgcgaagcgcttcagtgaaaaaatcataaggaaaagttgtaaatattattggtagtattcgtttggtaaagtagagggggtaatttttcccctttattttgttcatacattcttaaattgctttgcctctccttttggaaagctatacttcggagcactgttgagcgaaggctcaggccggcagcacgcagcacgctgtatttacgtatttaattttatatatttgtgcatacactactagggaagacttgaaaaaaacctaggaaatgaaaaaacgacacaggaagtcccgtatttactattttttccttccttttgatggggcagggcggaaatagaggataggataagcctactgcttagctgtttccgtctctacttcggtagttgtctcaattgtcgtttcagtattacctttagagccgctagacgatggttgagctatttgttgagggaaaactaagttcatgtaacacacgcataacccgattaaactcatgaatagcttgattgcaggaggctggtccattggagatggtgccttattttccttataggcaacgatgatgtcttcgtcggtgttcaggtagtagtgtacactctgaatcagggagaaccaggcaatgaacttgttcctcaagaaaatagcggccataggcatggattggttaaccacaccagatatgcttggtgtggcagaatatagtccttttggtggcgcaattttcttgtacctgtggtagaaagggagcggttgaactgttagtatatattggcaatatcagcaaatttgaaagaaaattgtcggtgaaaaacatacgaaacacaaaggtcgggccttgcaacgttattcaaagtcattgtttagttgaggaggtagcagcggagtatatgtattccttttttttgcctatggatgttgtaccatgcccattctgctcaagcttttgttaaaattatttttcagtattttttcttccatgttgcgcgttacgagaacagaagcgacagataaccgcaatcatacaactagcgctactgcggggtgtaaaaagcacaagaactaagccaagatcacaacagttat

SEQ ID NO: 3 - protein, 467 aa, Issachenkia orientalis
MSISEKYFPQEPQSPELKTAIPGPQSKAKLEELSAVYDTKAAYFVTDYYKSLGNYIVDADGNKLLDSYCQISSIALGYNNPALLKVAHSDEMAVALCNRPALACFPSTDYYEILKKGLLSVAPKGLDKVCTAHTGSDANEMAFKAAFLFQASKKRGDKPFTSEELESVMENKLPGTSDMVILSFEKGFHGRLFGSLSTTRSKAIHKLDIPAFEWPKAPFPQLKYPLDQFQAENKAEEERCLKALEEIIVNSPAKIAAAIIEPVQSEGGDNHASPEFFQGIREITKKHGVILIVDEVQTGGGASGKMWLHEHYGIVPDIMTFSKKMQNAGFFFSEAGLAGDQPFRQFNTWCGDPSKALIARTIIEEIKDKNLLTSVTETGDYLYSKLEAISAKYDKMINLRGKGRGFFIAFDAPTPELRNKFIAECKKLGLNIGGCGEQGVRLRPALVFEKKHADILASIIDQAFSKI

SEQ ID NO: 4 - DNA, 8446 bp, Artificial Sequence
  A synthetic oligonucleotide sequence
catctcccatcacataggaagcaacaggcgcgttggacttttaattttcgaggaccgcgaatccttacatcacacccaatcccccacaagtgatcccccacacaccatagcttcaaaatgtttctactccttttttactcttccagattttctcggactccgcgcatcgccgtaccacttcaaaacacccaagcacagcatactaaattccccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgtttttttttctctttcgatgacctcccattgatatttaagttaataaatggtcttcaatttctcaagtttcagtttcatttttcttgttctattacaactttttttacttcttgctcattagaaagaaagcatagcaatctaatctaagttttaattacaaatctagaatggggaaagagaagacgcatgtctctaggcccagactgaatagcaatatggatgccgacttatacgggtataagtgggccagagataatgttggtcagagcggggcgaccatctatcgtctttatggtaagccagacgcgccagaattattcctaaaacacggtaagggttctgttgctaatgacgttacggatgaaatggttaggctgaactggctgactgagtttatgcctttacctacaataaagcacttcataagaactccggatgacgcctggttactaacgactgctattcccggtaagaccgcattccaagtattggaggagtacccggacagtggggagaatatagttgacgcattagctgtttttttaaggaggctacattctataccagtatgtaactgcccctttaatagtgacagagtgtttcgtcttgctcaggcgcagagtcgtatgaataacgggttagttgatgcgtctgatttcgacgatgagagaaacggttggcccgttgaacaagtctggaaagagatgcacaaattgcttccttttagtcccgattctgtggtaacacacggtgacttctctttggataatctaatatttgatgaaggaaagttaatcgggtgcatagatgtgggccgtgtaggcatagcagatcgttaccaagatttagccattttatggaattgtctaggggagtttagtccctcactgcaaaaaaggttatttcagaaatacggcatcgacaaccccgacatgaacaagctgcagttccacctaatgttggacgaatttttttaattaatttaccagcttactatccttcttgaaaatatgcactctatatcttttagttcttaattgcaacacatagatttgctgtataacgaattttatgctatttttttaatttggagttcggtgatgaaagtgtcacagcgaatttcctcacatgtagggaccgaattgtttacaagttctctgtaccaccatggagacatcaaagattgaaaatctatggaaagatatggacggtagcaacaagaatatagcacgagccgcggagttcatttcgttacttttgatatcgctcacaactattgcgaagcgcttcagtgaaaaaatcataaggaaaagttgtaaatattattggtagtattcgtttggtaaagtagagggggtaatttttcccctttattttgttcatacattcttaaattgctttgcctctccttttggaaagctatacttcggagcactgttgagcgaaggctcagcggccgcgccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctgttgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaagaattcatgtctatttacttactgttcaccaaaacttgcctgcattaccagttgacgcaacctccgatgaagtcagaaagaaccttatggatatgtttagagatagacaagctttctccgaacatacttggaaaatgttattatccgtttgtagatcctgggccgcttggtgtaaacttaacaatagaaaatggtttcctgctgaaccagaagacgtcagagattacttactttacttacaagctagaggtttggctgttaaaactatccaacaacacttaggtcaattgaatatgttacacagaagatccggtttaccaagaccatccgattccaacgcagtttcccttgttatgagaagaattagaaaagaaaatgttgacgctggtgaaagagctaaacaagcattagcatttgaaagaaccgatttcgatcaagttagatccttaatggaaaattccgatagatgtcaagatattagaaacttagctttcttaggtattgcttacaacacattattaagaatcgctgaaattgctagaattagagttaaagatatttcaagaaccgatggcggtagaatgttaatccacattggcagaacaaaaaccttagtctccacagcaggcgtcgaaaaagcattatcattaggtgttactaaattagttgaacgttggatttccgtttccggtgttgcagatgacccaaacaactacttattctgtcgtgttagaaaaaatggtgttgccgctccttccgctacctcacaattatccacaagagcattagaaggcatttttgaagctacccacagacttatttatggtgcaaaagacgattccggtcaaagatatttagcttggtctggtcattccgctagagttggtgccgcaagagacatggcaagagctggtgtttctattcctgaaattatgcaagccggtggttggactaatgttaacattgttatgaactatatcagaaacttagattccgaaacaggtgctatggttagattacttgaagacggtgattaagctagctaagatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggagctccaattcgccctatagtgagtcgtattacaattcactggccgtcgttttacaacgtcgtgactgggaaaaccctggcgttacccaacttaatcgccttgcagcacatccccccttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcagggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagcgtggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttaggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgctttttttcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacgggcagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccaggggggaacgcctggtatctttatagtcctgtcgggtttcgccacctctgacttgagcgtcgatttttgtgatgctcgtcaggggggccgagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagctcggaattaaccctcactaaagggaacaaaagctgggtaccgggccccccctcgag

SEQ ID NO: 5 - DNA, 2622 bp, Aspergillus nidulans
tttttcttatcggagcgatataaaaagctgaagaaaggaggatagatgaaacagcatggcgcatagaaagtgttcaagctcactagtaaaggcgggaaatagaacattgagaacgtattttgataggaaacgaagataaagcggccgcataacttcgtataatgtatgctatacgaagttatccttacatcacacccaatcccccacaagtgatcccccacacaccatagcttcaaaatgtttctactccttttttactcttccagattttctcggactccgcgcatcgccgtaccacttcaaaacacccaagcacagcatactaaatttcccctctttcttcctctagggtggcgttaattacccgtactaaaggtttggaaaagaaaaaagagaccgcctcgtttctttttcttcgtcgaaaaaggcaataaaaatttttatcacgtttctttttcttgaaaaattttttttttgatttttttctctttcgatgacctcccattgatatttaagttaataaatggtcttcaatttctcaagtttcagtttcgtttttcttgttctattacaactttttttacttcttgctcattagaaagaaagcatagcaatctaatctaagttttaattacaaaatgccacaatcctgggaagaattggccgccgacaaacgtgcccgtttggctaaaaccattcctgacgaatggaaggttcaaactttgcctgccgaagattccgttattgatttcccaaagaagtccggtattttgtctgaggctgaattgaagattaccgaagcctctgctgctgatttggtctccaagttggccgctggtgagttgacttctgttgaagtcactttggctttttgtaagagagctgctattgctcaacaattaaccaactgtgctcacgaattcttcccagatgctgctttagctcaagctagagaattagatgaatactacgctaagcataagagaccagttggtccattacacggtttaccaatctctttaaaggaccaattgcgtgttaagggttacgaaacctccatgggttacatttcctggttaaacaaatacgatgaaggtgattccgtcttaaccaccatgttgagaaaagctggtgctgttttctacgttaagacctctgtcccacaaaccttgatggtctgtgaaaccgtcaacaacatcattggtagaactgtcaatccaagaaacaaaaattggtcctgtggtggttcttctggtggtgaaggtgctattgttggtattagaggtggtgttattggtgtcggtactgacattggtggttccattagagtcccagctgctttcaactttttatacggtttgagaccatctcacggtagattgccatatgctaaaatggctaactctatggaaggtcaagaaaccgttcactccgtcgttggtcctatcactcactccgtcgaagacttgagattgttcaccaaatctgtcttgggtcaagaaccttggaagtacgactctaaggtcatcccaatgccatggagacaatctgaatctgacatcattgcctctaagattaagaatggtggtttgaacattggttattacaatttcgacggtaacgtcttgccacacccaccaattttacgtggtgtcgaaactaccgttgccgctttggccaaggctggtcacaccgttactccatggactccatacaagcatgatttcggtcatgacttgatttcccacatctatgctgctgatggttctgccgacgtcatgagagacatttctgcctctggtgagccagccatccctaacattaaggacttgttgaacccaaatattaaggctgttaacatgaacgaattgtgggacactcatttacaaaagtggaactatcaaatggaatacttggaaaagtggcgtgaagctgaagaaaaagctggtaaggaattggacgctattatcgctccaattactcctaccgccgctgtcagacacgatcaattcagatactacggttacgcctccgttattaacttattggatttcacctctgttgtcgtcccagtcactttcgctgataagaatattgataagaagaacgaatcttttaaagctgtttccgaattggatgctttggttcaagaagaatacgacccagaggcttatcacggtgctcctgttgctgttcaagttattggtagaagattgtccgaagagagaactttggctatcgccgaagaagtcggtaaattgttgggtaacgtcgtcactccataaggaggttgataagacttttctagttgcatatcttttatatttaaatcttatctattagttaattttttgtaatttatccttatatatagtctggttattctaaaatatcatttcagtatctaaaaattcccctcttttttcagttatatcttaacaggcgataacttcgtataatgtatgctatacgaagttatgcggccgcgagaagatgcacctagctaaactaagtaaatctgtatactttttatacatgtaagactttttagctatttcattcttccctgaaccgttttgcgcgattctacggaatataccggcgaaataaaagagataaact

SEQ ID NO: 6 - DNA, 7755 bp, Artificial Sequence
  A synthetic oligonucleotide sequence
gctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagctcggaattaaccctcactaaagggaacaaaagctgggtaccgggccccccctcgagatctcccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagttttaaaacactaagaacttagtttcgaataaacacacataaaaacaaactagaatgacaattactccagctactcatgcaatttcaattaacccagctactggtgagcaattgtcagtcttgccatgggctggtgctgatgatattgagaacgctttgcaattggctgcagctggttttagagactggagagaaactaatattgattacagagctgaaaagttaagagatattggtaaggctttgagggctaggtctgaggaaatggcccagatgattactagagaaatgggaaaaccaattaaccaagctagagcagaggtcgctaagtctgctaacttgtgcgactggtatgctgagcacggtccagcaatgttgaaagctgagccaactttggttgaaaatcaacaagctgttattgagtatagacctttaggtactattttagctattatgccatggaactttccattgtggcaagttatgagaggtgcagttccaataattttagctggtaatggttatttgttgaagcatgctcctaacgtcatgggttgcgctcagttgatagcacaagtcttcaaggacgctggtattcctcaaggtgtctacggttggttgaacgctgataacgacggtgtctctcagatgatcaaagattcaagaattgctgctgttacagtcactggttctgtcagagctggtgctgctattggtgctcaagctggtgcagcattgaagaagtgcgtcttggaattaggtggttctgatccatttattgttttgaacgacgctgacttggagttggctgtcaaggcagctgtcgctggtcgttatcaaaacactggtcaagtttgcgctgctgctaagaggttcattattgaggaaggtattgcatcagcttttacagagagattcgtcgctgctgcagctgctttgaagatgggtgatccaagagacgaggagaacgctttaggtccaatggctaggttcgacttgagggacgaattgcaccaccaagttgagaagacattggctcaaggtgctagattgttgttgggtggtgagaagatggctggtgctggtaactactaccctccaacagtcttggcaaacgttacaccagagatgactgcttttcgtgaggagatgttcggtccagttgcagctattacaatagctaaggatgctgagcatgcattggaattggcaaatgattctgagtttggtttatctgcaactatttttactactgatgaaactcaagctagacagatggctgctagattggagtgtggaggtgtttttattaatggttattgtgcttctgatgcaagggtcgctttcggaggtgtcaagaagtctggttttggtagagaattatcacattttggtttgcacgaattttgtaatattcaaactgtctggaaagatagaatttaattaaacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgggcagacgcggccgccaccgcggtggagctccaattcgccctatagtgagtcgtattacaattcactggccgtcgttttacaacgtcgtgactgggaaaaccctggcgttacccaacttaatcgccttgcagcacatccccccttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcagggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagcgtggtgcactctcagtacaatctgctctgatgccgcatagttaagccagccccgacacccgccaacacccgctgacgcgccctgacgggcttgtctgctcccggcatccgcttacagacaagctgtgaccgtctccgggataacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgctttttttcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacgggcagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccaggggggaacgcctggtatctttatagtcctgtcgggtttcgccacctctgacttgagcgtcgatttttgtgatgctcgtcaggggggccgagcctatggaaaaacgccagcaacgcggcctttttacggttcctggcctttt

SEQ ID NO: 7 - protein, 462 aa, E. coli
MTITPATHAISINPATGEQLSVLPWAGADDIENALQLAAAGFRDWRETNIDYRAEKLRDIGKALRARSEEMAQMITREMGKPINQARAEVAKSANLCDWYAEHGPAMLKAEPTLVENQQAVIEYRPLGTILAIMPWNFPLWQVMRGAVPIILAGNGYLLKHAPNVMGCAQLIAQVFKDAGIPQGVYGWLNADNDGVSQMIKDSRIAAVTVTGSVRAGAAIGAQAGAALKKCVLELGGSDPFIVLNDADLELAVKAAVAGRYQNTGQVCAAAKRFIIEEGIASAFTERFVAAAAALKMGDPRDEENALGPMARFDLRDELHHQVEKTLAQGARLLLGGEKMAGAGNYYPPTVLANVTPEMTAFREEMFGPVAAITIAKDAEHALELANDSEFGLSATIFTTDETQARQMAARLECGGVFINGYCASDARVAFGGVKKSGFGRELSHFGLHEFCNIQTVWKDRI

SEQ ID NO: 8 - DNA, 7828 bp, Artificial Sequence
  A synthetic oligonucleotide sequence
gctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagctcggaattaaccctcactaaagggaacaaaagctgggtaccgggccccccctcgagatctcccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagttttaaaacactaagaacttagtttcgaataaacacacataaaaacaaatctagaatgcacatgtctgagactcctattaacactcaattattgattgctggtcaatggcaagacgctgctgacggtagaactttgccaattttagatccatctaatggtgaattgattggtaaagtcgcacacgcttctgtcgcagatttggataagacagttgctggtgcagctgctggttacgacgcttggagaaacttgactgcatgggatcgttacactattatgaggagggctgctgagttgttaagggagagagctgatgctattggtagaattatgtcaagagagcaaggtaagccattggctcaagctgttattgaaattaatgctgctgctggtgtcattgagtacttcggagaggaaggtagaaggttgaacgatgagttggtcccagctcgtgctccttttgtcgagcagagagtcttgcatcgtccaataggtgtcgttgcagctttcactccatggaacttcccaattaatcaaattgctagaaagttgggagctgctttaggtgctggttgtggtgtcatagtcaaggctccagaagacacacccgcttctcccgctgagttgattagatgtttctgtgatgctggtattcccgctggtgcagtctctttggtttacggtactcccgctgaaatttcagaatatttgattgctcatccagttgttagaaaaatttctttcactggttctacacccgttggtaagcacttggctgcattggctggtgcacaaatgaagccagtcacaatggaattgggtggtcacggtcccgttattgtctgcagagatgctgacttagatcaagctgctgaaagggttgcagctgctaagtttcgtaacgctggtcaagtttgcattgcaccaactagatttttattgcataaagatattgcttctgcttttattgaaaagtttaaagctcaaatgtcagctttaaagataggttgcggtttggatgagggtgtcacaatgggtccattaataaacgaaagaagagttaaggctttaactgaattggttgaagatgctagagctaagggtgctgagttgtggcaacccgatactgctttgccaaacaaaggtttctttttccctcctacattagttttgaaaccaacattggacatgagggtcatgcaagaagaaccatttggtccaattgcaattatggacgagttctctgagttggcagacgctgtcaaggaggctaatcgtttgccttacggtttagctgcttatgtttatactagatgtgagaggactgaaagattgttgggtgagaagttggaggctggtatggttgctattaaccaccacggtattggtacaccagaggtcccattcggtggtattaaggactctggttacggtacagaaggaggaccagctgcattgcgtgctcatactattattaagttggtttcttctttgcatgagagaccaaacttttaattaaacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgggcagacgcggccgccaccgcggtggagctccaattcgccctatagtgagtcgtattacaattcactggccgtcgttttacaacgtcgtgactgggaaaaccctggcgttacccaacttaatcgccttgcagcacatccccccttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcagggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagcgtggtgcactctcagtacaatctgctctgatgccgcatagttaagccagccccgacacccgccaacacccgctgacgcgccctgacgggcttgtctgctcccggcatccgcttacagacaagctgtgaccgtctccgggataacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgctttttttcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacgggcagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccaggggggaacgcctggtatctttatagtcctgtcgggtttcgccacctctgacttgagcgtcgatttttgtgatgctcgtcaggggggccgagcctatggaaaaacgccagcaacgcggcctttttacggttcctggcctttt

SEQ ID NO: 9 - protein, 486 aa, Acetobacter ghanensis
MHMSETPINTQLLIAGQWQDAADGRTLPILDPSNGELIGKVAHASVADLDKTVAGAAAGYDAWRNLTAWDRYTIMRRAAELLRERADAIGRIMSREQGKPLAQAVIEINAAAGVIEYFGEEGRRLNDELVPARAPFVEQRVLHRPIGVVAAFTPWNFPINQIARKLGAALGAGCGVIVKAPEDTPASPAELIRCFCDAGIPAGAVSLVYGTPAEISEYLIAHPVVRKISFTGSTPVGKHLAALAGAQMKPVTMELGGHGPVIVCRDADLDQAAERVAAAKFRNAGQVCIAPTRFLLHKDIASAFIEKFKAQMSALKIGCGLDEGVTMGPLINERRVKALTELVEDARAKGAELWQPDTALPNKGFFFPPTLVLKPTLDMRVMQEEPFGPIAIMDEFSELADAVKEANRLPYGLAAYVYTRCERTERLLGEKLEAGMVAINHHGIGTPEVPFGGIKDSGYGTEGGPAALRAHTIIKLVSSLHERPNF

SEQ ID NO: 10 - DNA, 7844 bp, Artificial Sequence
  A synthetic oligonucleotide sequence
gctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagctcggaattaaccctcactaaagggaacaaaagctgggtaccgggccccccctcgagatctcccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagttttaaaacactaagaacttagtttcgaataaacacacataaaaacaaatctagaatgactagaacttttgaattgggtgaattgattcgttctgataacttcattgatggtgcttggactccagcacaagacaacttgaggttcgctgtcactaacccagcttctggagagataattgctgaggtcgctgactcttctccagctgatgcaagagctgctactgatgcagctgcaagagctttgccagcttggagggctagattgccaaaagagagagctgcagtcttgcatcgttggcacgctttgataatggctaacttggatgcattgggtgctttaatatctttggagcaaggtaaacctttggctgagggtaagggtgaggtcgcttatggtgcttcttacgtcgcatggttcgcagaggaagcaacaagaatttacggtgatttgattcctcagcaacagaggggtaagaggatgactgctgtcaaagagccagtcggagtcgttgctgctattacaccatggaattttccattggcaatgattgcaagaaagatagcacccgctttggcagctggttgtactgttgtcgctaagccagctgaggacactccattgactgcttcagctttggtcttgttggctcacgaagctggtgttccacccggtgttttgaacttgattactgcatcccgtgatcatgctgtcgcagcagtcgctgagtggttgcatgacgctagagttagaaaaattacttttactggatcaactccagtcggtaagtacttggctagggaatctgctgaaactttaaagaagttatctttggagttgggtggtaatgctccatttattgtttttgacgatgctgacttggaggctgcagtcgctggtttgatggctgctaagtttcgtaacggtggtcagacttgtgtctgtccaaatcgtgtctacgtccaagctggtgtctacgagaggtttggtgctttgttggctgaaagggttggtgctttgaaggttgctccagcaactgatccagctgctcaaattggtccaatgattaattctagggcattggacaagattgctaggcacgtcgatgacgctgttgcacatggtgctagagtcttgactggtggtaagaggttggcagagttgggtccacactactacgctccaactgttttggctgacgcaacagcagcaatgcagttgaactcagaggaaactttcggtccaatagtcccattgtttcgtttcgaggacgaggctgaagcagttaacgctgctaacgacactccattcggtttagcagcttatttttattctgaaggtgtcaaaagaattgatagggtcgctagggctttggaagctggtattgttggtataaatgaaggtgcagtcgcttcagaagctgctccattcggtggtgttaaggaatctggttacggtagagaaggttctaaatatggtttggatgattatttgtctattaaatatttatgtcaaggtaatttagaataattaattaaacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgggcagacgcggccgccaccgcggtggagctccaattcgccctatagtgagtcgtattacaattcactggccgtcgttttacaacgtcgtgactgggaaaaccctggcgttacccaacttaatcgccttgcagcacatccccccttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcagggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagcgtggtgcactctcagtacaatctgctctgatgccgcatagttaagccagccccgacacccgccaacacccgctgacgcgccctgacgggcttgtctgctcccggcatccgcttacagacaagctgtgaccgtctccgggataacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgctttttttcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacgggcagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccaggggggaacgcctggtatctttatagtcctgtcgggtttcgccacctctgacttgagcgtcgatttttgtgatgctcgtcaggggggccgagcctatggaaaaacgccagcaacgcggcctttttacggttcctggcctttt

SEQ ID NO: 11 - protein, 490 aa, Paraburkholderia xenovorans
MTRTFELGELIRSDNFIDGAWTPAQDNLRFAVTNPASGEIIAEVADSSPADARAATDAAARALPAWRARLPKERAAVLHRWHALIMANLDALGALISLEQGKPLAEGKGEVAYGASYVAWFAEEATRIYGDLIPQQQRGKRMTAVKEPVGVVAAITPWNFPLAMIARKIAPALAAGCTVVAKPAEDTPLTASALVLLAHEAGVPPGVLNLITASRDHAVAAVAEWLHDARVRKITFTGSTPVGKYLARESAETLKKLSLELGGNAPFIVFDDADLEAAVAGLMAAKFRNGGQTCVCPNRVYVQAGVYERFGALLAERVGALKVAPATDPAAQIGPMINSRALDKIARHVDDAVAHGARVLTGGKRLAELGPHYYAPTVLADATAAMQLNSEETFGPIVPLFRFEDEAEAVNAANDTPFGLAAYFYSEGVKRIDRVARALEAGIVGINEGAVASEAAPFGGVKESGYGREGSKYGLDDYLSIKYLCQGNLE

SEQ ID NO: 12 - DNA, 7892 bp, Artificial Sequence
  A synthetic oligonucleotide sequence
gctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagctcggaattaaccctcactaaagggaacaaaagctgggtaccgggccccccctcgagatctcccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagttttaaaacactaagaacttagtttcgaataaacacacataaaaacaaatctagaatgatttatgctgctcccggtacacccggtgcagttgttacttttaaacctagatatggtaattacattggtggtgagtttgtcccaccagtcaagggtcaatacttcactaatacttcaccagttaatggtcaacctatagctgagttcccaaggtctacagctgaggacattgacaaggctttggatgcagctcacgctgcagcagaggcttggggtaggacttcagtccaagaacgttctaacattttgttgaagatagcagatagaattgaacaaaacttggaattattagctgtcactgagacttgggacaacggtaaagctgtcagagaaactttgaatgctgatattccattggctgctgaccactttcgttacttcgctggttgtattagggctcaagaaggttctgcagctgaaattaatgactcaacagttgcataccatattcacgaacctttgggtgttgtcggtcaaataattccatggaacttccctttgttgatggctgcttggaagttggctcccgctttggctgctggtaactgcgtcgtcttgaagccagcagagcaaactccattgggtatttgtgttttattggaattgattggagacttgttgccacccggtgtcttgaacgtcgtccaaggttttggtagggaagctggtgaagcattggctacttctaaaaggattgctaaaattgcatttactggttcaacaccagtcggttcacacattttgaagtgcgctgctgaaaatataattccatctactgttgaattgggtggtaaatcaccaaatatttattttgaagatattatgcaagctgaaccagcatttattgaaaaagctgctgaaggtttggttttagcttttttcaatcaaggtgaagtttgtacatgtccatcccgtgctttagttcaagaatcaatttatcccgcttttatggaagaagttttgaaaaaagttagagctataaagagaggtgacccattggacacagagacaatggttggtgcacaagcctctcaacaacagtacgaaaagattttgtcttatttggacatagctcaacaagaaggtgctgagttattggctggtggttcagttgaaaagttggaaggtaatttagcttctggttattacattcaaccaacattgttgaaaggtcataatggtatgagggttttccaagaggaaattttcggtccagtcgtcggtgtcactacattcaaggacgaagcagaggctttggctattgcaaacgacactgagtatggtttgggtgctggtttgtggacaagagatattaatcgtgcttatcgtatgggaaggggtattaaggctggtagggtttggacaaactgctaccacttgtatcccgctcacgctgctttcggtggttacaagaaatctggtgtcggtagagaaactcataagatgatgttggatcattatcagcaaactaagaacttgttagtttcttacgacattaacccattaggttttttctaattaattaaacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgggcagacgcggccgccaccgcggtggagctccaattcgccctatagtgagtcgtattacaattcactggccgtcgttttacaacgtcgtgactgggaaaaccctggcgttacccaacttaatcgccttgcagcacatccccccttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcagggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagcgtggtgcactctcagtacaatctgctctgatgccgcatagttaagccagccccgacacccgccaacacccgctgacgcgccctgacgggcttgtctgctcccggcatccgcttacagacaagctgtgaccgtctccgggataacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgctttttttcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacgggcagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccaggggggaacgcctggtatctttatagtcctgtcgggtttcgccacctctgacttgagcgtcgatttttgtgatgctcgtcaggggggccgagcctatggaaaaacgccagcaacgcggcctttttacggttcctggcctttt

SEQ ID NO: 13 - protein, 506 aa, Artificial Sequence
  A synthetic amino acid sequence
MIYAAPGTPGAVVTFKPRYGNYIGGEFVPPVKGQYFTNTSPVNGQPIAEFPRSTAEDIDKALDAAHAAAEAWGRTSVQERSNILLKIADRIEQNLELLAVTETWDNGKAVRETLNADIPLAADHFRYFAGCIRAQEGSAAEINDSTVAYHIHEPLGVVGQIIPWNFPLLMAAWKLAPALAAGNCVVLKPAEQTPLGICVLLELIGDLLPPGVLNVVQGFGREAGEALATSKRIAKIAFTGSTPVGSHILKCAAENIIPSTVELGGKSPNIYFEDIMQAEPAFIEKAAEGLVLAFFNQGEVCTCPSRALVQESIYPAFMEEVLKKVRAIKRGDPLDTETMVGAQASQQQYEKILSYLDIAQQEGAELLAGGSVEKLEGNLASGYYIQPTLLKGHNGMRVFQEEIFGPVVGVTTFKDEAEALAIANDTEYGLGAGLWTRDINRAYRMGRGIKAGRVWTNCYHLYPAHAAFGGYKKSGVGRETHKMMLDHYQQTKNLLVSYDINPLGFF

SEQ ID NO: 14 - DNA, 7760 bp, Artificial Sequence
  A synthetic oligonucleotide sequence
gctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagctcggaattaaccctcactaaagggaacaaaagctgggtaccgggccccccctcgagatctcccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagttttaaaacactaagaacttagtttcgaataaacacacataaaaacaaatctagaatgacaatgacaccagctactcatgctatttctgttgacccaacaactggtgaaacattggcaatgatgccatgggcttctgctgacgatattgagaacgctttgacattggctgctaggggtttcgaagaatggaaaagaactactgttgcttaccgtgctcaaaagttgagagatattggtcaagccttaagaaatagaactgaagaaatggctcactctatttcaagagaaatgggtaagccaattaggcaagctagagctgaggtcgctaagtcagctgctttgtgtgactggtacgctgaacacggtcccgctatgttgaacccagagccaacattggtcgagaaccaacaagctgtcattgcttatcgtccattgggtactattttggcagtcatgccatggaactttccattgtggcaagttttgagaggtgctattccaattttgttggctggtaatggttacttgttgaagcatgctccaaacgtcattggttgcgcagaaaggattgctcaaatttttgctgatgctggtattcccgctggtgtctacggatgggttaatgcatctaacgatggtgtttctcagatgataaacgatccaaggattgctgctgtcactgtcactggttctgttagagctggtgcagctattggtgctcaagctggtgctgctttgaagaaatgtgtcttggagttgggtggttcagacccattcattgttttgaacgacgcagacttggacttggctgttaaggctgctgtcgctggtcgttatcaaaacactggtcaagtctgcgcagctgcaaagagattcattgtcgaggaaggtatagctggtgagtttacagaaaggttcgtcgctgcagcttcagctttgaagatgggtgatccattggcagaagagaacgacttgggtccaatggctaggttcgacttgagagacgaattgcaccagcaagttgaggcttctatagctgagggagctagattattattgggaggtgaaaagttggctggtgaaggtaactattacgctataacagtcttgggtgacgtcactccagagatgactgcttttcgtcaagaattgttcggtccagtcgcagctattactgtcgcaaaaaacgcagagcatgctttacaattagctaatgactctgactttggtttgtctgctactttgtttactgctagagatgctttggctcaagaaatggcttcaaggttggaatgtggtggtgtcttcattaatggttactctgcttctgacccaagagtcgcttttggtggtgtcaagaagtctggatttggtagagagttatctcattttggtttgcatgaattttgtaatgttcaaacagtttggaaaaatagattgtaattaattaaacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgggcagacgcggccgccaccgcggtggagctccaattcgccctatagtgagtcgtattacaattcactggccgtcgttttacaacgtcgtgactgggaaaaccctggcgttacccaacttaatcgccttgcagcacatccccccttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcagggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagcgtggtgcactctcagtacaatctgctctgatgccgcatagttaagccagccccgacacccgccaacacccgctgacgcgccctgacgggcttgtctgctcccggcatccgcttacagacaagctgtgaccgtctccgggataacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgctttttttcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacgggcagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccaggggggaacgcctggtatctttatagtcctgtcgggtttcgccacctctgacttgagcgtcgatttttgtgatgctcgtcaggggggccgagcctatggaaaaacgccagcaacgcggcctttttacggttcctggcctttt

SEQ ID NO: 15 - protein, 462 aa, Citrobacter koseri
MTMTPATHAISVDPTTGETLAMMPWASADDIENALTLAARGFEEWKRTTVAYRAQKLRDIGQALRNRTEEMAHSISREMGKPIRQARAEVAKSAALCDWYAEHGPAMLNPEPTLVENQQAVIAYRPLGTILAVMPWNFPLWQVLRGAIPILLAGNGYLLKHAPNVIGCAERIAQIFADAGIPAGVYGWVNASNDGVSQMINDPRIAAVTVTGSVRAGAAIGAQAGAALKKCVLELGGSDPFIVLNDADLDLAVKAAVAGRYQNTGQVCAAAKRFIVEEGIAGEFTERFVAAASALKMGDPLAEENDLGPMARFDLRDELHQQVEASIAEGARLLLGGEKLAGEGNYYAITVLGDVTPEMTAFRQELFGPVAAITVAKNAEHALQLANDSDFGLSATLFTARDALAQEMASRLECGGVFINGYSASDPRVAFGGVKKSGFGRELSHFGLHEFCNVQTVWKNRL

SEQ ID NO: 16 - DNA, 7760 bp, Artificial Sequence
  A synthetic oligonucleotide sequence
gctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagctcggaattaaccctcactaaagggaacaaaagctgggtaccgggccccccctcgagatctcccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagttttaaaacactaagaacttagtttcgaataaacacacataaaaacaaatctagaatgactatttcttcagctacacacgctatttcagtcaacccagacactggagagcagttgtctgctttgccatgggcttctcagcacgacattgagagggcattgtctttagcagagcaaggtttcgctacttggaagaggacttctgtcgcttttcgtgctcagaagttgagagacttgggacaagccttaagagtccacggtgaggagatggctcaatgtattactagggaaatgggtaaaccagtcaagcaagctagagcagaggttgctaagtctgctgcattgtgcgactggtatgctgaacatggaccagctatgttggcaactgagcctacattggtcgaaaatcaacaagctgttattgaatatcgtccattaggtactattttagcagttatgccatggaactttccattgtggcaagttttgaggggtgctgttccaattttgttagctggtaatggttacttgttgaagcccgctccatgtgtcactggttgcgctcaaataatagctcaagttttcgctgaggcagagattccaatgggagtctacggttggttgaacgctgacaacgctggtgtctctgctgtcatacaagataggagaattgcagcagtcactgtcactggttctgttagggccggtgctgctattggtgctcaagctggcgccgctttgaagaaatgcgtcttggaattgggaggttcagacccatttattgttttgaacgacgctgatttggacttggcagtcaaagctgctgtcgctggtagataccagaacactggtcaagtctgcgctgctgctaaaagatttattatagaggaaggaattgcagacacttttactgagagatttgtcgccgctgctgccgccttgaagatgggaaaccctttggacgagcacaacgacttgggtccaatggctaggttcgacttgagggacgagttgcacgagcaagttcaagcaactttggctcaaggtgctcagttgttgttgggaggtgaaaaaatggctggtaagggtaactactacccagttactgttttagctaatgttacaccagaaatgactgcttttagagaagaattgttcggtcccgttgcagctattactgttgcaaaaaatgcagaacatgctttggagttagctaatgactcagatttcggtttgtctgctactttgtttactgcagacgaggttagagcaagggagatggctgctaggttagagtgtggtggtgttttcataaatggtttctctgcttctgacgctagggtcgctttcggtggagttaagaagtctggtttcggtagggaattgtcacatttcggtttacatgaattttgtaacgttcaaactgtttggaagaatagggtctaattaattaaacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgggcagacgcggccgccaccgcggtggagctccaattcgccctatagtgagtcgtattacaattcactggccgtcgttttacaacgtcgtgactgggaaaaccctggcgttacccaacttaatcgccttgcagcacatccccccttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcagggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagcgtggtgcactctcagtacaatctgctctgatgccgcatagttaagccagccccgacacccgccaacacccgctgacgcgccctgacgggcttgtctgctcccggcatccgcttacagacaagctgtgaccgtctccgggataacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgctttttttcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacgggcagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccaggggggaacgcctggtatctttatagtcctgtcgggtttcgccacctctgacttgagcgtcgatttttgtgatgctcgtcaggggggccgagcctatggaaaaacgccagcaacgcggcctttttacggttcctggcctttt

SEQ ID NO: 17 - protein, 462 aa, Citrobacter sedlakii
MTISSATHAISVNPDTGEQLSALPWASQHDIERALSLAEQGFATWKRTSVAFRAQKLRDLGQALRVHGEEMAQCITREMGKPVKQARAEVAKSAALCDWYAEHGPAMLATEPTLVENQQAVIEYRPLGTILAVMPWNFPLWQVLRGAVPILLAGNGYLLKPAPCVTGCAQIIAQVFAEAEIPMGVYGWLNADNAGVSAVIQDRRIAAVTVTGSVRAGAAIGAQAGAALKKCVLELGGSDPFIVLNDADLDLAVKAAVAGRYQNTGQVCAAAKRFIIEEGIADTFTERFVAAAAALKMGNPLDEHNDLGPMARFDLRDELHEQVQATLAQGAQLLLGGEKMAGKGNYYPVTVLANVTPEMTAFREELFGPVAAITVAKNAEHALELANDSDFGLSATLFTADEVRAREMAARLECGGVFINGFSASDARVAFGGVKKSGFGRELSHFGLHEFCNVQTVWKNRV

SEQ ID NO: 18 - DNA, 7760 bp, Artificial Sequence
  A synthetic oligonucleotide sequence
gctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagctcggaattaaccctcactaaagggaacaaaagctgggtaccgggccccccctcgagatctcccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagttttaaaacactaagaacttagtttcgaataaacacacataaaaacaaatctagaatgactatgatgactgctacacaagcattgtctgtcaaccccgctactggtcagactttagctgctatgccatgggctaacgctcaagaaattgagcacgctttgtcattggctgcttctggtttcaaaaaatggaagatgacttctgttgctcagagggctcagactttgagggacataggtcaagccttaagggctcacgctgaagaaatggcacaatgcattactagagaaatgggtaaaccaattaagcaagctagagctgaggtcactaagtctgctgcattgtgcgactggtatgctgagcatggaccagcaatgttgaactctgagcctactttggttgagaaccagcaagctgtcattgaatatcgtccattgggtgttattttagctattatgccatggaacttcccattgtggcaagttttgaggggtgctgttccaattttgttggctggtaactcttacttattgaagcatgctccaaacgtcactggttgcgcacagatgattgctaggattttgacagaagctggtactcccgctggtgtctacggttgggttaacgctaacaacgagggtgtctctcaaatgattaacgatccaagaattgctgcagttactgtcactggttctgttagggctggtgcagctattggtgctcaagttggcgctgctttgaagaagtgtgttttggaattaggtggatcagacccttttattgttttgaatgatgctgatttagagttggcagttaaggctgcagttgctggtcgttatcaaaacactggtcaagtttgcgctgctgctaaaaggttcattgtcgaggagggtattgcacaagcctttacagatcgtttcgtcgctgctgccgctgccttgaagatgggtgaccctttggtcgaggagaatgacttgggacctatggctaggttcgacttgagggatgaattacatcaacaagttcaagcctctgtcgctgagggtgctagattgttgttgggtggagaaaaaattgctggtgagggtaactactacgctgctacagtcttggctgacgtcactccagatatgacagcttttagacaagaattgttcggtcccgttgctgcaattactgttgcaagggacgcagctcatgcattggctttggctaatgattctgaatttggtttgtctgctacaatttttacagcagatgacacattggctgctgagatggcagctagattggagtgtggaggtgtttttataaatggttattcagcatctgatgctagagttgctttcggtggtgtcaagaagtctggtttcggaagagagttgtctcacttcggtttgcatgaattttgcaacgttcaaactgtttggaaaaacagagtttaattaattaaacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgggcagacgcggccgccaccgcggtggagctccaattcgccctatagtgagtcgtattacaattcactggccgtcgttttacaacgtcgtgactgggaaaaccctggcgttacccaacttaatcgccttgcagcacatccccccttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcagggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagcgtggtgcactctcagtacaatctgctctgatgccgcatagttaagccagccccgacacccgccaacacccgctgacgcgccctgacgggcttgtctgctcccggcatccgcttacagacaagctgtgaccgtctccgggataacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgctttttttcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacgggcagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccaggggggaacgcctggtatctttatagtcctgtcgggtttcgccacctctgacttgagcgtcgatttttgtgatgctcgtcaggggggccgagcctatggaaaaacgccagcaacgcggcctttttacggttcctggcctttt

SEQ ID NO: 19 - protein, 462 aa, Salmonella enterica
MTMMTATQALSVNPATGQTLAAMPWANAQEIEHALSLAASGFKKWKMTSVAQRAQTLRDIGQALRAHAEEMAQCITREMGKPIKQARAEVTKSAALCDWYAEHGPAMLNSEPTLVENQQAVIEYRPLGVILAIMPWNFPLWQVLRGAVPILLAGNSYLLKHAPNVTGCAQMIARILTEAGTPAGVYGWVNANNEGVSQMINDPRIAAVTVTGSVRAGAAIGAQVGAALKKCVLELGGSDPFIVLNDADLELAVKAAVAGRYQNTGQVCAAAKRFIVEEGIAQAFTDRFVAAAAALKMGDPLVEENDLGPMARFDLRDELHQQVQASVAEGARLLLGGEKIAGEGNYYAATVLADVTPDMTAFRQELFGPVAAITVARDAAHALALANDSEFGLSATIFTADDTLAAEMAARLECGGVFINGYSASDARVAFGGVKKSGFGRELSHFGLHEFCNVQTVWKNRV

SEQ ID NO: 20 - DNA, 7760 bp, Artificial Sequence
  A synthetic oligonucleotide sequence
gctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagctcggaattaaccctcactaaagggaacaaaagctgggtaccgggccccccctcgagatctcccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagttttaaaacactaagaacttagtttcgaataaacacacataaaaacaaatctagaatgactatatcaccagctactcatgctatttctattaacccttgtaacggtgagaggttgtctgcattgccatggtctactgcacaagatattgagcaagccttacagttggctgctgtcggtttcagagactggaagcaaactactgttgcttatagggctcaaaaattgagggacattggtcaagccttaagggttagaggtgaggagatggctcaatgtattactagagaaatgggtaaacctattaagcaagctagagctgaagtcgctaagtctgcagctttgtgcgactggtacgcagaacatggtccagcaatgttggatgcagagccaactcaagttgagaaccagcaagctgtcatagagtatcgtccattgggaactattttggctgtcatgccatggaacttcccattgtggcaagttttgagaggtgctgtcccaattttgttggctggtaacggttacttgttgaagcccgctccaaacgtcactggttgtgctaagattatttctcaagttttcacagatgcagctattccagctggtgtctacggttggttgaacgctgacaaccaaggtgtttctacattgattaatgacgcaagaattgctgcagtcactgtcactggttcagtcagagctggtgctgcaattggtgctcaagctggagccgctttgaagaagtgcgtcttggaattgggtggttctgacccattcattgtcttgaacgacgctgatttggatttggcagtccaagctgctgttgctggtcgttatcaaaattctggtcaagtttgcgcagcagctaagagatttattttagaagaaggtattgctgagtgctttacagagagattcgtcgccgctgctgccgctttaaagcaaggtgaccctttggctgaggagtcagaattaggtccaatggcaagggctgacttgagagacgagttgcaccagcaagttcaagctactattgcagagggtgctcagttgagattgggtggtgaaaaattgtctggtgctggtaacttctacccagcaactgttttaactaatgtcacaccagagatgactgcatttcgtgaggagttgtttggacccgttgcagctatagcagttgctaagtctgctgaacatgctttagagttggctaataactcagattttggtttgtctgctactttgttcactgctgatgaggcaagggctttggaaatgactgctaggttggagtgcggtggtgtttttattaatggattttctgcatcagatgctagagtcgctttcggaggagtcaagaagtctggttttggtagggaattgtctcacttcggtttgcacgagttctgcaacgtccaaactgtctggaaaaatagaagataattaattaaacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgggcagacgcggccgccaccgcggtggagctccaattcgccctatagtgagtcgtattacaattcactggccgtcgttttacaacgtcgtgactgggaaaaccctggcgttacccaacttaatcgccttgcagcacatccccccttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcagggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagcgtggtgcactctcagtacaatctgctctgatgccgcatagttaagccagccccgacacccgccaacacccgctgacgcgccctgacgggcttgtctgctcccggcatccgcttacagacaagctgtgaccgtctccgggataacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgctttttttcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacgggcagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccaggggggaacgcctggtatctttatagtcctgtcgggtttcgccacctctgacttgagcgtcgatttttgtgatgctcgtcaggggggccgagcctatggaaaaacgccagcaacgcggcctttttacggttcctggcctttt

SEQ ID NO: 21 - protein, 462 aa, Citrobacter amalonaticus
MTISPATHAISINPCNGERLSALPWSTAQDIEQALQLAAVGFRDWKQTTVAYRAQKLRDIGQALRVRGEEMAQCITREMGKPIKQARAEVAKSAALCDWYAEHGPAMLDAEPTQVENQQAVIEYRPLGTILAVMPWNFPLWQVLRGAVPILLAGNGYLLKPAPNVTGCAKIISQVFTDAAIPAGVYGWLNADNQGVSTLINDARIAAVTVTGSVRAGAAIGAQAGAALKKCVLELGGSDPFIVLNDADLDLAVQAAVAGRYQNSGQVCAAAKRFILEEGIAECFTERFVAAAAALKQGDPLAEESELGPMARADLRDELHQQVQATIAEGAQLRLGGEKLSGAGNFYPATVLTNVTPEMTAFREELFGPVAAIAVAKSAEHALELANNSDFGLSATLFTADEARALEMTARLECGGVFINGFSASDARVAFGGVKKSGFGRELSHFGLHEFCNVQTVWKNRR

SEQ ID NO: 22 - DNA, 7808 bp, Artificial Sequence
  A synthetic oligonucleotide sequence
gctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagctcggaattaaccctcactaaagggaacaaaagctgggtaccgggccccccctcgagatctcccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagttttaaaacactaagaacttagtttcgaataaacacacataaaaacaaatctagaatgcatacttatcccgatattaagttgttgattgacggtgaatggagggatgctgtctctggtaagactattgcagtctctgaccccgctacagacgaaataattggtgctatagcacatgctgagaaggaggacttggacttggctttggctgcagctgaaaggggttttagagtctggagggacacttcaccattcgagaggtctaagattatgagaagagctgcagacttgttgagagaaaggaaagataagattgcttacattatgacaagggaacaaggtaaaccattggcacagtctggtactgagattatgggtgctgcagacactattgactggttcgctgaagaggctaggagaacttacggtcaagttattccagcaagggctactggagtttctcagttggctattaagttaccagtcggtccagtcgctgctttcacaccatggaactttccaattaatcagattgttagaaagttgtctgccgctttggctactggatgctcaattattgttaaggctccagaagagacaccagcttcaccagctgaattgattagagcattcgttgatgctggtgtcccagctggtgtcattggtttggtttacggtgtcccatcagaaatatctgaatatttgataccacatcccgttattagaaaaatttcattcactggttctacaccaataggaaaacatttagctgctttagctggtaagcacatgaaaagagctactatggagttgggtggacacgctccagtcatgatctttgatgatgctgatattgaaaaagctattcaagttacttctttggcaaagtttcgtaacgctggtcaagtctgcgtcgcaccaactaggttcttggttcaagatggagttgctgacagattcttggagggtttcgttgaggctacaaaggctattaaagttggtaatggtttggaggacgacgtcgtcatgggacctttggctaatgaaagaagaataccagctttagaaggtttgattaacgacgctgtctctcacggtgctgacttgaagactggtggtagaagaataggtaacaaaggtaatttctttgagcctacagtcttggctaacgtcccaactactgctaaaattatgaacgacgaaccattcggtcccgttgctattattaatcgtttttcttcttttgacgatgctattactgaagctaacagattgccatttggtttagcttcttatgcttttactggttcagttaagactgcacacgctttgggacagcaagttgaagctggtatgttgactattaatcacaacggattagctttgcccgaagtccctttcggtggtattaaggactctggttacggtactgaaggtggttctgaggctgtccaagcctatttggaaactaaatttgtttcacaaatgtcataattaattaaacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgggcagacgcggccgccaccgcggtggagctccaattcgccctatagtgagtcgtattacaattcactggccgtcgttttacaacgtcgtgactgggaaaaccctggcgttacccaacttaatcgccttgcagcacatccccccttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcagggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagcgtggtgcactctcagtacaatctgctctgatgccgcatagttaagccagccccgacacccgccaacacccgctgacgcgccctgacgggcttgtctgctcccggcatccgcttacagacaagctgtgaccgtctccgggataacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgctttttttcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacgggcagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccaggggggaacgcctggtatctttatagtcctgtcgggtttcgccacctctgacttgagcgtcgatttttgtgatgctcgtcaggggggccgagcctatggaaaaacgccagcaacgcggcctttttacggttcctggcctttt

SEQ ID NO: 23 - protein, 479 aa, Agrobacterium deltaense
HMHTYPDIKLLIDGEWRDAVSGKTIAVSDPATDEIIGAIAHAEKEDLDLALAAAERGFRVWRDTSPFERSKIMRRAADLLRERKDKIAYIMTREQGKPLAQSGTEIMGAADTIDWFAEEARRTYGQVIPARATGVSQLAIKLPVGPVAAFTPWNFPINQIVRKLSAALATGCSIIVKAPEETPASPAELIRAFVDAGVPAGVIGLVYGVPSEISEYLIPHPVIRKISFTGSTPIGKHLAALAGKHMKRATMELGGHAPVMIFDDADIEKAIQVTSLAKFRNAGQVCVAPTRFLVQDGVADRFLEGFVEATKAIKVGNGLEDDVVMGPLANERRIPALEGLINDAVSHGADLKTGGRRIGNKGNFFEPTVLANVPTTAKIMNDEPFGPVAIINRFSSFDDAITEANRLPFGLASYAFTGSVKTAHALGQQVEAGMLTINHNGLALPEVPFGGIKDSGYGTEGGSEAVQAYLETKFVSQMS

SEQ ID NO: 24 - DNA, 7757 bp, Artificial Sequence
  A synthetic oligonucleotide sequence
gctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagctcggaattaaccctcactaaagggaacaaaagctgggtaccgggccccccctcgagatctcccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagttttaaaacactaagaacttagtttcgaataaacacacataaaaacaaatccatatgacgattacccctgctacccatgcgatttctataaacccagctaccggtgaacaactttcagtattgccttgggccggtgccgacgacatcgagaatgcacttcaattagctgccgctggtttcagagattggagagagacaaatattgattatcgggcagaaaaactcagagatattggtaaagcgttgagggttcgttctgaggaaatggcgcaaatgatcactagagaaatgggtaagcccataaatcaagctagagcagaagtggctaagtccgcaaacttatgtgattggtacgcagaacatggtccagcgatgctaaaagctgagccaactttggttgaaaatcagcaagccgtgatagaatataggccgctaggcacaattctggcaatcatgccctggaactggccactttggcaagtcatgagaggggcagttccaatcattctcgccggaaacgggtacttgctaaagcatgcacctaatgtcatgggctgtgcacaattgatagcacaagtttttaaagatgcgggaatacctcagggagtttatggttggttgaatgctgacaatgatggtgtctcacaaatgattaaagacagtcgaatcgctgctgttactgttacaggttctgttcgtgcaggggctgccatcggagcacaggctggagccgcattgaaaaagtgtgttcttgaattggggggtagtgatccattcattgtactcaatgatgccgacttagaattagcggtaaaagctgcagtggcaggcagataccaaaatactggtcaagtttgcgctgctgccaagaggttcattatagaagagggcatcgctagtgcctttacagaaagatttgttgcagctgccgcagctctgaaaatgggagatcctagagatgaggaaaacgcgcttggtcccatggcccggttcgatctgagagatgaattacaccatcaggttgaaaagacattagcacagggtgctaggctactattgggtggagaaaagatggcaggcgcaggaaactactatcctccaaccgtgttggcaaatgtcacgccagagatgacagcatttcgagaggaaatgtttggtccagtcgcagcaattacaattgccaaagatgctgagcacgcattagaattggctaacgactcagaatttggtctatccgctactatattcactactgatgaaacacaagctagacagatggcagctaggttagagtgcggtggcgtatttattaatgggtattgtgccagcgatgcaagagtcgcatttggtggagtaaaaaaatctggattcggtagagagttatcgcatttcggtttgcatgaattttgtaatattcaaaccgtttggaaagaccgtatttaaggatccacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgggcagacgcggccgccaccgcggtggagctccaattcgccctatagtgagtcgtattacaattcactggccgtcgttttacaacgtcgtgactgggaaaaccctggcgttacccaacttaatcgccttgcagcacatccccccttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcagggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacataaccgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagcgtggtgcactctcagtacaatctgctctgatgccgcatagttaagccagccccgacacccgccaacacccgctgacgcgccctgacgggcttgtctgctcccggcatccgcttacagacaagctgtgaccgtctccgggataacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgctttttttcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacgggcagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccaggggggaacgcctggtatctttatagtcctgtcgggtttcgccacctctgacttgagcgtcgatttttgtgatgctcgtcaggggggccgagcctatggaaaaacgccagcaacgcggcctttttacggttcctggcctttt

SEQ ID NO: 25 - protein, 462 aa, Artificial Sequence
  A synthetic amino acid sequence
MTITPATHAISINPATGEQLSVLPWAGADDIENALQLAAAGFRDWRETNIDYRAEKLRDIGKALRVRSEEMAQMITREMGKPINQARAEVAKSANLCDWYAEHGPAMLKAEPTLVENQQAVIEYRPLGTILAIMPWNWPLWQVMRGAVPIILAGNGYLLKHAPNVMGCAQLIAQVFKDAGIPQGVYGWLNADNDGVSQMIKDSRIAAVTVTGSVRAGAAIGAQAGAALKKCVLELGGSDPFIVLNDADLELAVKAAVAGRYQNTGQVCAAAKRFIIEEGIASAFTERFVAAAAALKMGDPRDEENALGPMARFDLRDELHHQVEKTLAQGARLLLGGEKMAGAGNYYPPTVLANVTPEMTAFREEMFGPVAAITIAKDAEHALELANDSEFGLSATIFTTDETQARQMAARLECGGVFINGYCASDARVAFGGVKKSGFGRELSHFGLHEFCNIQTVWKDRI

SEQ ID NO: 26 - DNA, 7844 bp, Artificial Sequence
  A synthetic oligonucleotide sequence
gctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagctcggaattaaccctcactaaagggaacaaaagctgggtaccgggccccccctcgagatctcccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagttttaaaacactaagaacttagtttcgaataaacacacataaaaacaaatctagaatgactagaacttttgaattgggtgaattgattcgttctgataacttcattgatggtgcttggactccagcacaagacaacttgaggttcgctgtcactaacccagcttctggagagataattgctgaggtcgctgactcttctccagctgatgcaagagctgctactgatgcagctgcaagagctttgccagcttggagggctagattgccaaaagagagagctgcagtcttgcatcgttggcacgctttgataatggctaacttggatgcattgggtgctttaatatctttggagcaaggtaaacctttggctgagggtaagggtgaggtcgcttatggtgcttcttacgtcgcatggttcgcagaggaagcaacaagaatttacggtgatttgattcctcagcaacagaggggtaagaggatgactgctgtcaaagagccagtcggagtcgttgctgctattacaccatggaattggccattggcaatgattgcaagaaagatagcacccgctttggcagctggttgtactgttgtcgctaagccagctgaggacactccattgactgcttcagctttggtcttgttggctcacgaagctggtgttccacccggtgttttgaacttgattactgcatcccgtgatcatgctgtcgcagcagtcgctgagtggttgcatgacgctagagttagaaaaattacttttactggatcaactccagtcggtaagtacttggctagggaatctgctgaaactttaaagaagttatctttggagttgggtggtaatgctccatttattgtttttgacgatgctgacttggaggctgcagtcgctggtttgatggctgctaagtttcgtaacggtggtcagacttgtgtctgtccaaatcgtgtctacgtccaagctggtgtctacgagaggtttggtgctttgttggctgaaagggttggtgctttgaaggttgctccagcaactgatccagctgctcaaattggtccaatgattaattctagggcattggacaagattgctaggcacgtcgatgacgctgttgcacatggtgctagagtcttgactggtggtaagaggttggcagagttgggtccacactactacgctccaactgttttggctgacgcaacagcagcaatgcagttgaactcagaggaaactttcggtccaatagtcccattgtttcgtttcgaggacgaggctgaagcagttaacgctgctaacgacactccattcggtttagcagcttatttttattctgaaggtgtcaaaagaattgatagggtcgctagggctttggaagctggtattgttggtataaatgaaggtgcagtcgcttcagaagctgctccattcggtggtgttaaggaatctggttacggtagagaaggttctaaatatggtttggatgattatttgtctattaaatatttatgtcaaggtaatttagaataattaattaaacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgggcagacgcggccgccaccgcggtggagctccaattcgccctatagtgagtcgtattacaattcactggccgtcgttttacaacgtcgtgactgggaaaaccctggcgttacccaacttaatcgccttgcagcacatccccccttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcagggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagcgtggtgcactctcagtacaatctgctctgatgccgcatagttaagccagccccgacacccgccaacacccgctgacgcgccctgacgggcttgtctgctcccggcatccgcttacagacaagctgtgaccgtctccgggataacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgctttttttcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacgggcagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccaggggggaacgcctggtatctttatagtcctgtcgggtttcgccacctctgacttgagcgtcgatttttgtgatgctcgtcaggggggccgagcctatggaaaaacgccagcaacgcggcctttttacggttcctggcctttt

SEQ ID NO: 27 - protein, 490 aa, Artificial Sequence
  A synthetic amino acid sequence
MTRTFELGELIRSDNFIDGAWTPAQDNLRFAVTNPASGEIIAEVADSSPADARAATDAAARALPAWRARLPKERAAVLHRWHALIMANLDALGALISLEQGKPLAEGKGEVAYGASYVAWFAEEATRIYGDLIPQQQRGKRMTAVKEPVGVVAAITPWNWPLAMIARKIAPALAAGCTVVAKPAEDTPLTASALVLLAHEAGVPPGVLNLITASRDHAVAAVAEWLHDARVRKITFTGSTPVGKYLARESAETLKKLSLELGGNAPFIVFDDADLEAAVAGLMAAKFRNGGQTCVCPNRVYVQAGVYERFGALLAERVGALKVAPATDPAAQIGPMINSRALDKIARHVDDAVAHGARVLTGGKRLAELGPHYYAPTVLADATAAMQLNSEETFGPIVPLFRFEDEAEAVNAANDTPFGLAAYFYSEGVKRIDRVARALEAGIVGINEGAVASEAAPFGGVKESGYGREGSKYGLDDYLSIKYLCQGNLE

SEQ ID NO: 28 - DNA, 7863 bp, Artificial Sequence
  A synthetic oligonucleotide sequence
tagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgggcagacgcggccgccaccgcggtggagctccaattcgccctatagtgagtcgtattacaattcactggccgtcgttttacaacgtcgtgactgggaaaaccctggcgttacccaacttaatcgccttgcagcacatccccccttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcagggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagcgtggtgcactctcagtacaatctgctctgatgccgcatagttaagccagccccgacacccgccaacacccgctgacgcgccctgacgggcttgtctgctcccggcatccgcttacagacaagctgtgaccgtctccgggataacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgctttttttcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacgggcagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccaggggggaacgcctggtatctttatagtcctgtcgggtttcgccacctctgacttgagcgtcgatttttgtgatgctcgtcaggggggccgagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagctcggaattaaccctcactaaagggaacaaaagctgggtaccgggccccccctcgagatctcccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagttttaaaacactaagaacttagtttcgaataaacacacataaaaacaaataacttagtttcgaataaacacacataaaaacaaatctagaatgcacatgtctgagactcctattaacactcaattattgattgctggtcaatggcaagacgctgctgacggtagaactttgccaattttagatccatctaatggtgaattgattggtaaagtcgcacacgcttctgtcgcagatttggataagacagttgctggtgcagctgctggttacgacgcttggagaaacttgactgcatgggatcgttacactattatgaggagggctgctgagttgttaagggagagagctgatgctattggtagaattatgtcaagagagcaaggtaagccattggctcaagctgttattgaaattaatgctgctgctggtgtcattgagtacttcggagaggaaggtagaaggttgaacgatgagttggtcccagctcgtgctccttttgtcgagcagagagtcttgcatcgtccaataggtgtcgttgcagctttcactccatggaactggccaattaatcaaattgctagaaagttgggagctgctttaggtgctggttgtggtgtcatagtcaaggctccagaagacacacccgcttctcccgctgagttgattagatgtttctgtgatgctggtattcccgctggtgcagtctctttggtttacggtactcccgctgaaatttcagaatatttgattgctcatccagttgttagaaaaatttctttcactggttctacacccgttggtaagcacttggctgcattggctggtgcacaaatgaagccagtcacaatggaattgggtggtcacggtcccgttattgtctgcagagatgctgacttagatcaagctgctgaaagggttgcagctgctaagtttcgtaacgctggtcaagtttgcattgcaccaactagatttttattgcataaagatattgcttctgcttttattgaaaagtttaaagctcaaatgtcagctttaaagataggttgcggtttggatgagggtgtcacaatgggtccattaataaacgaaagaagagttaaggctttaactgaattggttgaagatgctagagctaagggtgctgagttgtggcaacccgatactgctttgccaaacaaaggtttctttttccctcctacattagttttgaaaccaacattggacatgagggtcatgcaagaagaaccatttggtccaattgcaattatggacgagttctctgagttggcagacgctgtcaaggaggctaatcgtttgccttacggtttagctgcttatgtttatactagatgtgagaggactgaaagattgttgggtgagaagttggaggctggtatggttgctattaaccaccacggtattggtacaccagaggtcccattcggtggtattaaggactctggttacggtacagaaggaggaccagctgcattgcgtgctcatactattattaagttggtttcttctttgcatgagagaccaaacttttaattaaacaggccccttttcctttgtcgatatcatgtaat

SEQ ID NO: 29 - protein, 486 aa, Artificial Sequence
  A synthetic amino acid sequence
MHMSETPINTQLLIAGQWQDAADGRTLPILDPSNGELIGKVAHASVADLDKTVAGAAAGYDAWRNLTAWDRYTIMRRAAELLRERADAIGRIMSREQGKPLAQAVIEINAAAGVIEYFGEEGRRLNDELVPARAPFVEQRVLHRPIGVVAAFTPWNWPINQIARKLGAALGAGCGVIVKAPEDTPASPAELIRCFCDAGIPAGAVSLVYGTPAEISEYLIAHPVVRKISFTGSTPVGKHLAALAGAQMKPVTMELGGHGPVIVCRDADLDQAAERVAAAKFRNAGQVCIAPTRFLLHKDIASAFIEKFKAQMSALKIGCGLDEGVTMGPLINERRVKALTELVEDARAKGAELWQPDTALPNKGFFFPPTLVLKPTLDMRVMQEEPFGPIAIMDEFSELADAVKEANRLPYGLAAYVYTRCERTERLLGEKLEAGMVAINHHGIGTPEVPFGGIKDSGYGTEGGPAALRAHTIIKLVSSLHERPNF

SEQ ID NO: 30 - DNA, 7808 bp, Artificial Sequence
  A synthetic oligonucleotide sequence
gctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagctcggaattaaccctcactaaagggaacaaaagctgggtaccgggccccccctcgagatctcccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagttttaaaacactaagaacttagtttcgaataaacacacataaaaacaaatctagaatgcatacttatcccgatattaagttgttgattgacggtgaatggagggatgctgtctctggtaagactattgcagtctctgaccccgctacagacgaaataattggtgctatagcacatgctgagaaggaggacttggacttggctttggctgcagctgaaaggggttttagagtctggagggacacttcaccattcgagaggtctaagattatgagaagagctgcagacttgttgagagaaaggaaagataagattgcttacattatgacaagggaacaaggtaaaccattggcacagtctggtactgagattatgggtgctgcagacactattgactggttcgctgaagaggctaggagaacttacggtcaagttattccagcaagggctactggagtttctcagttggctattaagttaccagtcggtccagtcgctgctttcacaccatggaactggccaattaatcagattgttagaaagttgtctgccgctttggctactggatgctcaattattgttaaggctccagaagagacaccagcttcaccagctgaattgattagagcattcgttgatgctggtgtcccagctggtgtcattggtttggtttacggtgtcccatcagaaatatctgaatatttgataccacatcccgttattagaaaaatttcattcactggttctacaccaataggaaaacatttagctgctttagctggtaagcacatgaaaagagctactatggagttgggtggacacgctccagtcatgatctttgatgatgctgatattgaaaaagctattcaagttacttctttggcaaagtttcgtaacgctggtcaagtctgcgtcgcaccaactaggttcttggttcaagatggagttgctgacagattcttggagggtttcgttgaggctacaaaggctattaaagttggtaatggtttggaggacgacgtcgtcatgggacctttggctaatgaaagaagaataccagctttagaaggtttgattaacgacgctgtctctcacggtgctgacttgaagactggtggtagaagaataggtaacaaaggtaatttctttgagcctacagtcttggctaacgtcccaactactgctaaaattatgaacgacgaaccattcggtcccgttgctattattaatcgtttttcttcttttgacgatgctattactgaagctaacagattgccatttggtttagcttcttatgcttttactggttcagttaagactgcacacgctttgggacagcaagttgaagctggtatgttgactattaatcacaacggattagctttgcccgaagtccctttcggtggtattaaggactctggttacggtactgaaggtggttctgaggctgtccaagcctatttggaaactaaatttgtttcacaaatgtcataattaattaaacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgggcagacgcggccgccaccgcggtggagctccaattcgccctatagtgagtcgtattacaattcactggccgtcgttttacaacgtcgtgactgggaaaaccctggcgttacccaacttaatcgccttgcagcacatccccccttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcagggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagcgtggtgcactctcagtacaatctgctctgatgccgcatagttaagccagccccgacacccgccaacacccgctgacgcgccctgacgggcttgtctgctcccggcatccgcttacagacaagctgtgaccgtctccgggataacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgctttttttcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacgggcagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccaggggggaacgcctggtatctttatagtcctgtcgggtttcgccacctctgacttgagcgtcgatttttgtgatgctcgtcaggggggccgagcctatggaaaaacgccagcaacgcggcctttttacggttcctggcctttt

SEQ ID NO: 31 - protein, 478 aa, Artificial Sequence
  A synthetic amino acid sequence
MHTYPDIKLLIDGEWRDAVSGKTIAVSDPATDEIIGAIAHAEKEDLDLALAAAERGFRVWRDTSPFERSKIMRRAADLLRERKDKIAYIMTREQGKPLAQSGTEIMGAADTIDWFAEEARRTYGQVIPARATGVSQLAIKLPVGPVAAFTPWNWPINQIVRKLSAALATGCSIIVKAPEETPASPAELIRAFVDAGVPAGVIGLVYGVPSEISEYLIPHPVIRKISFTGSTPIGKHLAALAGKHMKRATMELGGHAPVMIFDDADIEKAIQVTSLAKFRNAGQVCVAPTRFLVQDGVADRFLEGFVEATKAIKVGNGLEDDVVMGPLANERRIPALEGLINDAVSHGADLKTGGRRIGNKGNFFEPTVLANVPTTAKIMNDEPFGPVAIINRFSSFDDAITEANRLPFGLASYAFTGSVKTAHALGQQVEAGMLTINHNGLALPEVPFGGIKDSGYGTEGGSEAVQAYLETKFVSQMS

SEQ ID NO: 32 - DNA, 2319 bp, Artificial Sequence
  A synthetic oligonucleotide sequence
ctagcgtaccgtatcacagtatagtctaatattccgtatcttattgtatcctatcctattcgatcctattgtatttcagtgcaccattttaatttctattgctataatgtccttattagttgccactgtgaggtgaccaatggacgagggcgagccgttcagaagccgcgaagggtgttcttcccatgaatttcttaaggagggcggctcagctccgagagtgaggcgagacgtctcggtcagcgtatcccccttcctcggcttttacaaatgatgcgctcttaatagtgtgtcgttatccttttggcattgacgggggagggaaattgattgagcgcatccatatttttgcggactgctgaggacaatggtggtttttccgggtggcgtgggctacaaatgatacgatggtttttttcttttcggagaaggcgtataaaaaggacacggagaacccatttattctaaaaacagttgagcttctttaattattttttgatataatattctattattatatattttcttcccaataaaacaaaataaaacaaaacacagcaaaacacaaaagaattcgcccttacatatgataacttcgtataatgtatgctatacgaagttatcatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaactttttttttttcttggtgcacggacatgtttttaaaggaagtactctataccagttattcttcacaaatttaattgctggagaatagatcttcaacgctttaataaagtagtttgtttgtcaaggatggcgtcatacaaagaaagatcagaatcacacacttcccctgttgctaggagacttttctccatcatggaggaaaagaagtctaacctttgtgcatcattggatattactgaaactgaaaagcttctctctattttggacactattggtccttacatctgtctagttaaaacacacatcgatattgtttctgattttacgtatgaaggaactgtgttgcctttgaaggagcttgccaagaaacataattttatgatttttgaagatagaaaatttgctgatattggtaacactgttaaaaatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactggatcattatgactccaggggttggtttagatgacaaaggtgatgcacttggtcaacaatatagaactgttgatgaagttgtaaagactggaacggatatcataattgttggtagaggtttgtacggtcaaggaagagatcctatagagcaagctaaaagataccaacaagctggttggaatgcttatttaaacagatttaaatgattcttacacaaagatttgatacatgtacactagtttaaataagcatgaaaagaattacacaagcaaaaaaaaaaaaataaatgaggtactttacgttcacctacaaccaaaaaaactagatagagtaaaatcttaagatttagaaaaagttgtttaacaaaggctttagtatgtgaatttttaatgtagcaaagcgataactaataaacataaacaaaagtatggttttctttatcagtcaaatcattatcgattgattgttccgcgtatctgcagataacttcgtataatgtatgctatacgaagttatagatccgcggccgcttaacatctgaatgtaaaatgaacattaaaatgaattactaaactttacgtctactttacaatctataaactttgtttaatcatataacgaaatacactaatacacaatcctgtacgtatgtaatacttttatccatcaaggattgagaaaaaaaagtaatgattccctgggccattaaaacttagacccccaagcttggataggtcactctctattttcgtttctcccttccctgatagaagggtgagagct

SEQ ID NO: 33 - DNA, 3252 bp, Artificial Sequence
  A synthetic oligonucleotide sequence
ctagcgtaccgtatcacagtatagtctaatattccgtatcttattgtatcctatcctattcgatcctattgtatttcagtgcaccattttaatttctattgctataatgtccttattagttgccactgtgaggtgaccaatggacgagggcgagccgttcagaagccgcgaagggtgttcttcccatgaatttcttaaggagggcggctcagctccgagagtgaggcgagacgtctcggtcagcgtatcccccttcctcggcttttacaaatgatgcgctcttaatagtgtgtcgttatccttttggcattgacgggggagggaaattgattgagcgcatccatatttttgcggactgctgaggacaatggtggtttttccgggtggcgtgggctacaaatgatacgatggtttttttcttttcggagaaggcgtataaaaaggacacggagaacccatttattctaaaaacagttgagcttctttaattattttttgatataatattctattattatatattttcttcccaataaaacaaaataaaacaaaacacagcaaaacacaaaagaattcgcccttacatatggataacttcgtataatgtatgctatacgaagttatgctgcaacggcaacatcaatgtccacgtttacacacctacatttatatctatatttatatttatatttatttatttatgctacttagcttctatagttagttaatgcactcacgatattcaaaattgacacccttcaactactccctactattgtctactactgtctactactcctctttactatagctgctcccaataggctccaccaataggctctgtcaatacattttgcgccgccacctttcaggttgtgtcactcctgaaggaccatattgggtaatcgtgcaatttctggaagagagtgccgcgagaagtgaggcccccactgtaaatcctcgagggggcatggagtatggggcatggaggatggaggatggggggggggggggaaaataggtagcgaaaggacccgctatcaccccacccggagaactcgttgccgggaagtcatatttcgacactccggggagtctataaaaggcgggttttgtcttttgccagttgatgttgctgagaggacttgtttgccgtttcttccgatttaacagtatagaatcaaccactgttaattatacacgttatactaacacaacaaaaacaaaaacaacgacaacaacaacaacaatgtttgctttctactttctcaccgcatgcaccactttgaagggtgttttcggagtttctccgagttacaatggtcttggtctcaccccacagatgggttgggacagctggaatacgtttgcctgcgatgtcagtgaacagctacttctagacactgctgatagaatttctgacttggggctaaaggatatgggttacaagtatgtcatcctagatgactgttggtctagcggcagggattccgacggtttcctcgttgcagacaagcacaaatttcccaacggtatgggccatgttgcagaccacctgcataataacagctttcttttcggtatgtattcgtctgctggtgagtacacctgtgctgggtaccctgggtctctggggcgtgaggaagaagatgctcaattctttgcaaataaccgcgttgactacttgaagtatgataattgttacaataaaggtcaatttggtacaccagacgtttcttaccaccgttacaaggccatgtcagatgctttgaataaaactggtaggcctattttctattctctatgtaactggggtcaggatttgacattttactggggctctggtatcgccaattcttggagaatgagcggagatattactgctgagttcacccgtccagatagcagatgtccctgtgacggtgacgaatatgattgcaagtacgccggtttccattgttctattatgaatattcttaacaaggcagctccaatggggcaaaatgcaggtgttggtggttggaacgatctggacaatctagaggtcggagtcggtaatttgactgacgatgaggaaaaggcccatttctctatgtgggcaatggtaaagtccccacttatcattggtgccgacgtgaatcacttaaaggcatcttcgtactcgatctacagtcaagcctctgtcatcgcaattaatcaagatccaaagggtattccagccacaagagtctggagatattatgtttcagacaccgatgaatatggacaaggtgaaattcaaatgtggagtggtccgcttgacaatggtgaccaagtggttgctttattgaatggaggaagcgtagcaagaccaatgaacacgaccttggaagagattttctttgacagcaatttgggttcaaaggaactgacatcgacttgggatatttacgacttatgggccaacagagttgacaactctacggcgtctgctatccttgaacagaataaggcagccaccggtattctctacaatgctacagagcagtcttataaagacggtttgtctaagaatgatacaagactgtttggccagaaaattggtagtctttctccaaatgctatacttaacacaactgttccagctcatggtatcgccttctataggttgagaccctcggcttaagctcaatgttgagcaaagcaggacgagaaaaaaaaaaataatgattgttaagaagttcatgaaaaaaaaaaggaaaaatactcaaatacttataacagagtgattaaataataaacggcagtataccctatcaggtattgagatagttttatttttgtaggtatataatctgaagcctttgaactattttctcgtatatatcatggagtatacattgcattagcaacattgcatactagttcataacttcgtataatgtatgctatacgaagttattaattaacaagggcgatttctgcagatatcggccggccccatggagatccgcggccgcttaacatctgaatgtaaaatgaacattaaaatgaattactaaactttacgtctactttacaatctataaactttgtttaatcatataacgaaatacactaatacacaatcctgtacgtatgtaatacttttatccatcaaggattgagaaaaaaaagtaatgattccctgggccattaaaacttagacccccaagcttggataggtcactctctattttcgtttctcccttccctgatagaagggtgagagct

SEQ ID NO: 34 - DNA, 9629 bp, Artificial Sequence
  A synthetic oligonucleotide sequence
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggtcgaggagtccatcggttcctgtcagatgggatactcttgacgtggaaaattcaaacagaaaaaaaaccccaataatgaaaaataacactacgttatatccgtggtatcctctatcgtatcgtatcgtagcgtatcgtagcgtaccgtatcacagtatagtctaatattccgtatcttattgtatcctatcctattcgatcctattgtatttcagtgcaccattttaatttctattgctataatgtccttattagttgccactgtgaggtgaccaatggacgagggcgagccgttcagaagccgcgaagggtgttcttcccatgaatttcttaaggagggcggctcagctccgagagtgaggcgagacgtctcggtcagcgtatcccccttcctcggcttttacaaatgatgcgctcttaatagtgtgtcgttatccttttggcattgacgggggagggaaattgattgagcgcatccatatttttgcggactgctgaggacaatggtggtttttccgggtggcgtgggctacaaatgatacgatggtttttttcttttcggagaaggcgtataaaaaggacacggagaacccatttattctaaaaacagttgagcttctttaattattttttgatataatattctattattatatattttcttcccaataaaacaaaataaaacaaaacacagcaaaacacaaaaaggatccatgtctaatttacttactgttcaccaaaacttgcctgcattaccagttgacgcaacctccgatgaagtcagaaagaaccttatggatatgtttagagatagacaagctttctccgaacatacttggaaaatgttattatccgtttgtagatcctgggccgcttggtgtaaacttaacaatagaaaatggtttcctgctgaaccagaagacgtcagagattacttactttacttacaagctagaggtttggctgttaaaactatccaacaacacttaggtcaattgaatatgttacacagaagatccggtttaccaagaccatccgattccaacgcagtttcccttgttatgagaagaattagaaaagaaaatgttgacgctggtgaaagagctaaacaagcattagcatttgaaagaaccgatttcgatcaagttagatccttaatggaaaattccgatagatgtcaagatattagaaacttagctttcttaggtattgcttacaacacattattaagaatcgctgaaattgctagaattagagttaaagatatttcaagaaccgatggcggtagaatgttaatccacattggcagaacaaaaaccttagtctccacagcaggcgtcgaaaaagcattatcattaggtgttactaaattagttgaacgttggatttccgtttccggtgttgcagatgacccaaacaactacttattctgtcgtgttagaaaaaatggtgttgccgctccttccgctacctcacaattatccacaagagcattagaaggcatttttgaagctacccacagacttatttatggtgcaaaagacgattccggtcaaagatatttagcttggtctggtcattccgctagagttggtgccgcaagagacatggcaagagctggtgtttctattcctgaaattatgcaagccggtggttggactaatgttaacattgttatgaactatatcagaaacttagattccgaaacaggtgctatggttagattacttgaagacggtgattaagttaattaacatctgaatgtaaaatgaacattaaaatgaattactaaactttacgtctactttacaatctataaactttgtttaatcatataacgaaatacactaatacacaatcctgtacgtatgtaatacttttatccatcaaggattgagaaaaaaaagtaatgattccctgggccattaaaacttagacccccaagcttggataggtcactctctattttcgtttctcccttccctgatagaagggtgatatgtaattaagaataatatataattttataataaaagaattcggcagatctggatcgatcccccgggctgcatgcaacggcaacatcaatgtccacgtttacacacctacatttatatctatatttatatttatatttatttatttatgctacttagcttctatagttagttaatgcactcacgatattcaaaattgacacccttcaactactccctactattgtctactactgtctactactcctctttactatagctgctcccaataggctccaccaataggctctgtcaatacattttgcgccgccacctttcaggttgtgtcactcctgaaggaccatattgggtaatcgtgcaatttctggaagagagtgccgcgagaagtgaggcccccactgtaaatcctcgagggggcatggagtatggggcatgaaggatggaggatggggggggggggggaaaataggtagcgaaaggacccgctatcaccccacccggagaactcgttgccgggaagtcatatttcgacactccggggagtctataaaaggcgggttttgtcttttgccagttgatgttgctgagaggacttgtttgccgtttcttccgatttaacagtatagaatcaaccactgttaattatacacgttatactaacacaacaaaaacaaaaacaacgacaacaacaacaacctgcaggaaatgcttttgcaagctttccttttccttttggctggttttgcagccaaaatatctgcatcaatgacaaacgaaactagcgatagacctttggtccacttcacacccaacaagggctggatgaatgacccaaatgggttgtggtacgatgaaaaagatgccaaatggcatctgtactttcaatacaacccaaatgacaccgtatggggtacgccattgttttggggccatgctacttccgatgatttgactaattgggaagatcaacccattgctatcgctcccaagcgtaacgattcaggtgctttctctggctccatggtggttgattacaacaacacgagtgggtttttcaatgatactattgatccaagacaaagatgcgttgcgatttggacttataacactcctgaaagtgaagagcaatacattagctattctcttgatggtggttacacttttactgaataccaaaagaaccctgttttagctgccaactccactcaattcagagatccaaaggtgttctggtatgaaccttctcaaaaatggattatgacggctgccaaatcacaagactacaaaattgaaatttactcctctgatgacttgaagtcctggaagctagaatctgcatttgccaatgaaggtttcttaggctaccaatacgaatgtccaggtttgattgaagtcccaactgagcaagatccttccaaatcttattgggtcatgtttatttctatcaacccaggtgcacctgctggcggttccttcaaccaatattttgttggatccttcaatggtactcattttgaagcgtttgacaatcaatctagagtggtagattttggtaaggactactatgccttgcaaactttcttcaacactgacccaacctacggttcagcattaggtattgcctgggcttcaaactgggagtacagtgcctttgtcccaactaacccatggagatcatccatgtctttggtccgcaagttttctttgaacactgaatatcaagctaatccagagactgaattgatcaatttgaaagccgaaccaatattgaacattagtaatgctggtccctggtctcgttttgctactaacacaactctaactaaggccaattcttacaatgtcgatttgagcaactcgactggtaccctagagtttgagttggtttacgctgttaacaccacacaaaccatatccaaatccgtctttgccgacttatcactttggttcaagggtttagaagatcctgaagaatatttgagaatgggttttgaagtcagtgcttcttccttctttttggaccgtggtaactctaaggtcaagtttgtcaaggagaacccatatttcacaaacagaatgtctgtcaacaaccaaccattcaagtctgagaacgacctaagttactataaagtgtacggcctactggatcaaaacatcttggaattgtacttcaacgatggagatgtggtttctacaaatacctacttcatgaccaccggtaacgctctaggatctgtgaacatgaccactggtgtcgataatttgttctacattgacaagttccaagtaagggaagtaaaatagcctgcaggcacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctgaattcccgggccttaccgtcgacgaatttcagcattttcatttcaaggcgatattatgtttcactaaactcaggacaggaatatactaagaataactacaacatacacacaacataagccaagatggatcaacttaactaccaagaacaacaacaatttcaaaagatcgttgaacaaaagcaaatggctgatttcatgaggctatgaattcgcccttgatctgggtgtatactgcacaacctcattgttcgggaatttgattctcatctcacatacaggcctgtagtattgcgccctctccttctccttctccttctccttctccaagagagacttctctctcatcgccctcgtcatcaatggctgctcgctgtattgtcgttggagcatctcccgatacttctgcaactgtgataaactcatctcaggtgacccatccgattctgtatcggtgtctccatctggggctacatctcgggccagtctagatttaaactttgcagaaccttcactttgggggatatacactagtgtctctcccgtgactacatcaccgacaccctcaactgtaccattattattgtcattgttttcctctaagttctcgctttggtcttcatccatctctccttcgggtgctgtatcactcttgatgatttctctaaccctaatacggagactgtgattgcctgaaataatacccacatctttcaacttctgatgaagtgaatctccagagatgaccttcatcagcacttgcacatcaaccacatcaccctccttttgagcatccctcatgattccatagactacatcccgtagcgtctccttgttcttgtacttcttaacaacagtctcgccacagacatggcccctgataatcacctcctgtctctcctcatggccatcctggtcgccattgtcttcgtcgctcggctcaattgccaatgtagcaccctgtggaagattgcttagtctgtatggaacagactcatcaactcttttgccattatgcattaacttgtactttcgcccttggctaagttgaaaatgtttacatccttcttcaagtacattggacatgattgtccctgcattgacatttgtccggtaagtcctaaacccactcgctagattcactgtaggcatattcaatcacgttccgtttgaaaaaaaggaaaccaatttattatctccagaaatagttggcgtcttgcatcttgtttggtcttgatctttcgtgttttttttttttctgtctttttttttctcctctctccaactttttgatttttagtgtaccaaatcgcactgcttatccacattcatcataaagagggggggagaagaggggcagaaaataaaaggccatgtcacgtgcctgtgcatttatttgtgtgtgtgtcacgtgctcaaaatgtcttttttttacgtttttaacattttccctttctgtagttgaatccatttgcatgagtcgtacatgatgtttgctgtatttacgttaagacactaattcaaatgacaaacagctattattcttagccattaatgcatttttgcaaatctttaactggatttaactatggctaggtgaatttgttctggacatcattgccttgacttgttttagtgccgatgtccttatcacttacactcgtaacacaacacaacagcagctaatgttgttgtgtatcgcttgacccttaataactgattcttttttgatgaatgttaagaagaaacaaacaagaaaataaaatcaaaacaggcttcttttgacctctttcaagagaaggttttcttggttgtttcatataccaagatctgaatatcttctattattatacaaaccactgattatacaaatctattcatcgacagtatgagctacgaaaacacactgataaagagagtcatttcttccccttctttttctttttctttttcttcttcttcttagtatccccatcttcattaactccaccaagtagatcctctacaccccccatggccgttaaaaaatgttcacgaaagaaatccatatcattattcttaccatccattaaactgtttagatagatggtgatcatctcccttgcattgtctatatcttcaacgtcgagtaaatgcgacgcaatggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 35 - DNA, 4770 bp, Artificial Sequence
  A synthetic oligonucleotide sequence
cgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgttaaccccatgcactcaactatccccgtaaacatagtagtaggctataggctttcttctcgtcatcacagagaagccatatagagacatatacatattaatacatggacttctaggcttactttctcacatacttttcacgtactttctcacatatttactaatacacaatttttacattatgtgcatttgtaaattgcaatgtaggattttcctcattgacaaggacaaaccctcatttgggaggaaaagagaaaataacttctccgtttttttttgtttttttttctcagatgagccgccactgttttttttttttttgcatccccctttttccaatttgtcatgactataaaagcgcgaattccccccccccccttgtctcccgtttctttcctgccatgcgagattgctcattcaatttgaccttctcttgatgtggcaatttatctccaaaactgacagcttaagcttctttccaatgttggtttctggtaaacgaatgccatagagaaaggctggagatacagagacaggtctgtggttgtttcccacacgggtttgtttttgattctcctcgacatggtttgtctgctgaatatatataaatattcatacacctctatctaactatctatctataaaaaatcgcaatgtagtagctatattaacattaactatggcaataataacaatagcaatactgaaaacaatgatgttattattattatcgtgggcaataccaaaaaccgaggtcattcaaaagggcgcacattggtggaaaaacaatacttgcgcggaaaaaaaaaaattaatatctgccaaatcctaaactgcaaatcatggattgcaattctccgttatctatataaggctaggaggttgccaatcggtatataatagttgggggggaggaggaggaggattctctttagattatcacttgatacaaatcaatcggttatcttttattgaaaagtcaagacacaaaccaaccaactaaacgagcggccgcgagtccatcggttcctgtcagatgggatactcttgacgtggaaaattcaaacagaaaaaaaaccccaataatgaaaaataacactacgttatatccgtggtatcctctatcgtatcgtatcgtagcgtatcgtagcgtaccgtatcacagtatagtctaatattccgtatcttattgtatcctatcctattcgatcctattgtatttcagtgcaccattttaatttctattgctataatgtccttattagttgccactgtgaggtgaccaatggacgagggcgagccgttcagaagccgcgaagggtgttcttcccatgaatttcttaaggagggcggctcagctccgagagtgaggcgagacgtctcggtcagcgtatcccccttcctcggcttttacaaatgatgcgctcttaatagtgtgtcgttatccttttggcattgacgggggagggaaattgattgagcgcatccatatttttgcggactgctgaggacaatggtggtttttccgggtggcgtgggctacaaatgatacgatggtttttttcttttcggagaaggcgtataaaaaggacacggagaacccatttattctaaaaacagttgagcttctttaattattttttgatataatattctattattatatattttcttcccaataaaacaaaataaaacaaaacacagcaaaacacaaaaagctagctaaaatgagagttgattccgagatcattgtcaagaaggaaacacaagatgaaaacctctaccaatctttagcagaaagatccaaacatgaagagttcttaagaaaggccgttgacttacttgtcgaaagagttgttttcggcagatcaaccagatcttccaaggttgttgagtgggctgcaccagacgagattaagaaagcaattgacttaaagccacgtttgggtcctgcttctcacgatgagttgttggcctttatggcaaacgttgctaggtattctgtcaatactggtcatccatacttcgttaaccagttattctcatccgttgatccatatggtttagttggtcaatggttaaccgacgcattgaacccatccgtttacacctttgaagtcgctcctgtctttaccttgatggaggaagaggttttgcgtgaaatgcgtaagattgtcggttggcctgagggcgaaggtgatggtatcttctgcccaggtggttctatcgccaacggttacgctatttcctgtgctagacatcacttttacccagaggtcaagtataagggtgttcatgctgttcctaagttagttttgtttacctccgaactcgcgcactacagtaccaaaaagatggctgctttcatgggtattggttccgataactgtgttaacattaagaccgatgatgttggtaagatgaacatcgttgatttagaaatgaagatcaagattgctattgacaacaagtgtaccccattcatggttacagctacctctggtactaccgttttcggtgctttcgacccattagtcgcaatctccgatctttgcaagaagtacaatctgtggttgcatgtcgatgctgcttggggtggtggtgctctcatgtctaagaagcacagacaccttttgaacggtatcgaattagccgactctgttacttggaaccctcataagcttttagccgctccacaacaatgttccacctttttgaccagacacaagaaagttctatccgaaggtcattcatccaatgcaaagtacttattccaaaaagacaagttttacgacacatcctacgacactggtgataaacatattcaatgtggtagaagagcagatgttcttaagttctggttcatgtggaaggctaagggtactgaaggtttcgagaaacacgttgacaagctttttgataacgcaaagtacttcttagaccacattaagcagagagaaggtttccaattggttattgcagaaccacaatgcaccaatatcatgttctggtacatcccaaagtgcctgagaggttgtgaaaatgatgcagactactatgagagattacataaagttgcgcctaagatcaaggaaagaatgatcaaggaaggttcaatgatggtcacctaccaaccacaaggtgatcttgttaacttctttagaattgtttttcagaactctgcattggaccataaggacatggtttacttcgcaaatgaattcgaaagattgggctccgatatgatcgtttaattaattaatttattttactagtttatttttgctcctgagaataggattacaaacacttaaagtctttaattacaactatatataatattctgttggttttcttgaattggttcgctgcgattcatgcctcccattcaccaaaggtggagtgggaaataacggttttactgcggtaattagcagaggcaagaacaggatacactttttgatgataaatctgtattatagtcgagcctatttaggaaatcaaattttcttgtgtttacttttcaaataaataatgttcgaaaatttttactttactccttcatttaactataccagacgttatatcatcaacaccttctgaccatatacagctcaagatgtttaagagtctgttaaattttttcaatccatttcatggagtaccaggaggtgctacaaaaggaattcatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaactttttttttttcttggtgcacggacatgtttttaaaggaagtactctataccagttattcttcacaaatttaattgctggagaatagatcttcaacgctttaataaagtagtttgtttgtcaaggatggcgtcatacaaagaaagatcagaatcacacacttcccctgttgctaggagacttttctccatcatggaggaaaagaagtctaacctttgtgcatcattggatattactgaaactgaaaagcttctctctattttggacactattggtccttacatctgtctagttaaaacacacatcgatattgtttctgattttacgtatgaaggaactgtgttgcctttgaaggagcttgccaagaaacataattttatgatttttgaagatagaaaatttgctgatattggtaacactgttaaaaatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactccgc

SEQ ID NO: 36 - protein, 456 aa, Danaus plexippus
KVVEWAAPDEIKKAIDLKPRLGPASHDELLAFMANVARYSVNTGHPYFVNQLFSSVDPYGLVGQWLTDALNPSVYTFEVAPVFTLMEEEVLREMRKIVGWPEGEGDGIFCPGGSIANGYAISCARHHFYPEVKYKGVHAVPKLVLFTSELAHYSTKKMAAFMGIGSDNCVNIKTDDVGKMNIVDLEMKIKIAIDNKCTPFMVTATSGTTVFGAFDPLVAISDLCKKYNLWLHVDAAWGGGALMSKKHRHLLNGIELADSVTWNPHKLLAAPQQCSTFLTRHKKVLSEGHSSNAKYLFQKDKFYDTSYDTGDKHIQCGRRADVLKFWFMWKAKGTEGFEKHVDKLFDNAKYFLDHIKQREGFQLVIAEPQCTNIMFWYIPKCLRGCENDADYYERLHKVAPKIKERMIKEGSMMVTYQPQGDLVNFFRIVFQNSALDHKDMVYFANEFERLGSDMIV

SEQ ID NO: 37 - DNA, 5134 bp, Artificial Sequence
  A synthetic oligonucleotide sequence
cgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattctttgaaggagcttgccaagaaacataattttatgatttttgaagatagaaaatttgctgatattggtaacactgttaaaaatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactggatcattatgactccaggggttggtttagatgacaaaggtgatgcacttggtcaacaatatagaactgttgatgaagttgtaaagactggaacggatatcataattgttggtagaggtttgtacggtcaaggaagagatcctatagagcaagctaaaagataccaacaagctggttggaatgcttatttaaacagatttaaatgattcttacacaaagatttgatacatgtacactagtttaaataagcatgaaaagaattacacaagcaaaaaaaaaaaaataaatgaggtactttacgttcacctacaaccaaaaaaactagatagagtaaaatcttaagatttagaaaaagttgtttaacaaaggctttagtatgtgaatttttaatgtagcaaagcgataactaataaacataaacaaaagtatggttttctttatcagtcaaatcattatcgattgattgttccgcgtatctgcagatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaactttttttttttcttggtgcacggacatgtttttaaaggaagtactctataccagttattcttcacaaatttaattgctggagaatagatcttcaacgcgtttaaacagcaatttgaggaaggaataggagaaggagaagcaatttctaggaaagagcaaggtgtgcaacagcatgctctgaatgatattttcagcaatagttcagttgaagaacctgttggcgtatctacatcacttcctacaaacaacaccacgaattgcgtccgtggtgacgcaactacgaatggcattgtcaatgccaatgccagtgcacatacacgtgcaagtcccaccggttccctgcccggctatggtagagacaagaaggacgataccggcatcgacatcaacagtttcaacagcaatgcgtttggcgtcgacgcgtcgatggggctgccgtatttggatttggacgggctagatttcgatatggatatggatatggatatggatatggagatgaatttgaatttagatttgggtcttgatttggggttggaattaaaaggggataacaatgagggttttcctgttgatttaaacaatggacgtgggaggtgattgatttaacctgatccaaaaggggtatgtctattttttagagtgtgtctttgtgtcaaattatggtagaatgtgtaaagtagtataaactttcctctcaaatgacgaggtttaaaacaccccccgggtgagccgagccgagaatggggcaattgttcaatgtgaaatagaagtatcgagtgagaaacttgggtgttggccagccaagggggaaggaaaatggcgcgaatgctcaggtgagattgttttggaattgggtgaagcgaggaaatgagcgacccggaggttgtgactttagtggcggaggaggacggaggaaaagccaagagggaagtgtatataaggggagcaatttgccaccaggatagaattggatgagttataattctactgtatttattgtataatttatttctccttttatatcaaacacattacaaaacacacaaaacacacaaacaaacacatctagataaaatgagggttgactccaaaatcatcgtcaaaaaggaaacccaagatgaaaacttgtaccaatctttggctgaaagatccaagcatgaagagtttttgagaaaagccgttgatttgttggttgaaagagttgtttttggtagatccactagatcctccaaggtcgttgaatgggcagctccagatgaaatcaaaaaggccatcgatcttaagcctaggttaggcccagcttcccatgatgaattgttagcctttatggcaaatgttgcaagatattccgtcaatactggtcacccatacttcgttaatcaattgttttcatctgttgatccatacggtctagttggtcaatggttgaccgatgccttaaacccttctgtctatacctttgaagttgctccagtctttacattgatggaggaagaggttttgagagaaatgaggaaaatcgttggttggccagaaggtgagggtgatggcatcttttgtccaggtggttctatcgccaacggttacgctatttcttgtgctcgtcatcacttctacccagaagttaagtacaaaggtgttcatgctgtccctaagttagtcttgtttacctctgaattagctcactattctacaaagaaaatggctgcattcatgggtattggttccgacaattgtgtcaatatcaaaacagacgacgttggtaagatgaacattgttgacctagaaatgaagattaagatcgcaattgacaacaagtgtacccctttcatggttacagctacatcaggtactactgttttcggtgcttttgacccacttgtcgcaatctccgatttgtgtaagaagtacaacttatggctacatgtcgatgcggcatggggtggtggtgcgttaatgtcaaagaagcatagacatcttctaaacggtatcgaattagcagactccgttacctggaatccacacaaattgttagctgctcctcaacaatgttccacattccttaccagacacaagaaggtcctgtctgaaggtcattcttccaacgccaaatacttgtttcagaaggacaagttttacgacacttcttatgacactggtgacaaacatattcagtgtggtagaagagcagatgttttgaagttctggtttatgtggaaggcaaagggtacggagggtttcgagaagcacgttgataagttgtttgacaatgctaagtacttcctagatcacattaagcaaagggaaggtttccaattggtcattgcagaacctcagtgtaccaacatcatgttctggtatatcccaaagtgtctaagaggttgtgaaaatgatgcagactactatgaaagattgcataaagtcgctccaaagatcaaagaaagaatgatcaaagaaggttctatgatggtcacctaccaaccacaaggtgatttggtcaatttctttagaatcgttttccagaattctgctttagatcataaggatatggtctactttgcaaacgaattcgaaagattaggttccgacatgatcgtttaattaacatctgaatgtaaaatgaacattaaaatgaattactaaactttacgtctactttacaatctataaactttgtttaatcatataacgaaatacactaatacacaatcctgtacgtatgtaatacttttatccatcaaggattgagaaaaaaaagtaatgattccctgggccattaaaacttagacccccaagcttggataggtcactctctattttcgtttctcccttccctgatagaagggtgatatgtaattaagaataatatataattttataataaaagcggccgccaacttggttcaaaccaagaaagccgtcgtgtactttgcgtattttcaacttcttcccttcaactccaaccttataacccaatccgacagttagcatacagtagaaaatccataagttaattgcaaaaaccacataattcccaatgactggcacaatttgcatgaacggcgcaaaaccaacacctatccttcttgacttgtggacaacttctccggcatcggctccagtttctggtttaggcgacactttcaaggtacccttgttgactaggttctcagcaacatactcctcctcgataactttgaccgtctttgtgccaaacacattgttcaatagtttggacgctcgcataccgaagacagactcgtccatccaatagcatgtactccgaatgctctcaatcaacggtatcaaatcgcttggtgtctttggaggcggtgttctaggtttcatcttcgccttcccgtttcctgtttcttggacttcaaaatacgggtcatatgcatcttcattggtgaacctgaacagtttgggccgagtagtgccaaacaccttcaacagtataatcttcactccttggccgattagaaacatgcttatatgtttgccggtattcttaaagtgtatactggagcccttttaatcacattttttttacttgtcagtctccatacggagtttaatgtccttatatcgatcttcatcagctccaacgggacaggaatcaataaccttccttgcctgtcccaaaaagaatgaattttttttcaaaagctttacgatgcataccacaaaaggaagattattcccacatgttccagaagtgtgcggagatacaaagggttcatgaaaacgtgaatcttctaaaaacttagcacaacaataaaaatctacaatgttacagtaagtattattttctttttgtcgacacactccaacggttagatttccaagtattcaatccaatgtattacttgtcagacagccatccactcccatcttagaacatcacttccgaaccgc

SEQ ID NO: 38 - DNA, 4685 bp, Artificial Sequence
  A synthetic oligonucleotide sequence
ctggcgaaagggggatgtgctgcaaggcgattaagttgggtaacgccagggttttcccagtcacgacgttgtaaaacgacggccagtgaattcgttaaccccatgcactcaactatccccgtaaacatagtagtaggctataggctttcttctcgtcatcacagagaagccatatagagacatatacatattaatacatggacttctaggcttactttctcacatacttttcacgtactttctcacatatttactaatacacaatttttacattatgtgcatttgtaaattgcaatgtaggattttcctcattgacaaggacaaaccctcatttgggaggaaaagagaaaataacttctccgtttttttttgtttttttttctcagatgagccgccactgttttttttttttttgcatccccctttttccaatttgtcatgactataaaagcgcgaattccccccccccccttgtctcccgtttctttcctgccatgcgagattgctcattcaatttgaccttctcttgatgtggcaatttatctccaaaactgacagcttaagcttctttccaatgttggtttctggtaaacgaatgccatagagaaaggctggagatacagagacaggtctgtggttgtttcccacacgggtttgtttttgattctcctcgacatggtttgtctgctgaatatatataaatattcatacacctctatctaactatctatctataaaaaatcgcaatgtagtagctatattaacattaactatggcaataataacaatagcaatactgaaaacaatgatgttattattattatcgtgggcaataccaaaaaccgaggtcattcaaaagggcgcacattggtggaaaaacaatacttgcgcggaaaaaaaaaaattaatatctgccaaatcctaaactgcaaatcatggattgcaattctccgttatctatataaggctaggaggttgccaatcggtatataatagttgggggggaggaggaggaggattctctttagattatcacttgatacaaatcaatcggttatcttttattgaaaagtcaagacacaaaccaaccaactaaacgagcggccgcgagtccatcggttcctgtcagatgggatactcttgacgtggaaaattcaaacagaaaaaaaaccccaataatgaaaaataacactacgttatatccgtggtatcctctatcgtatcgtatcgtagcgtatcgtagcgtaccgtatcacagtatagtctaatattccgtatcttattgtatcctatcctattcgatcctattgtatttcagtgcaccattttaatttctattgctataatgtccttattagttgccactgtgaggtgaccaatggacgagggcgagccgttcagaagccgcgaagggtgttcttcccatgaatttcttaaggagggcggctcagctccgagagtgaggcgagacgtctcggtcagcgtatcccccttcctcggcttttacaaatgatgcgctcttaatagtgtgtcgttatccttttggcattgacgggggagggaaattgattgagcgcatccatatttttgcggactgctgaggacaatggtggtttttccgggtggcgtgggctacaaatgatacgatggtttttttcttttcggagaaggcgtataaaaaggacacggagaacccatttattctaaaaacagttgagcttctttaattattttttgatataatattctattattatatattttcttcccaataaaacaaaataaaacaaaacacagcaaaacacaaaaagctagctaaaatgagagttgattccaagatcattgtcaagaaagagactcaagacgaaaacttgtatcaatccttgcaaggttgttgagtgggctgcaccagacgagattaagaaagcaattgacttaaagccacgtttgggtcctgcttctcacgatgagttgttggcctttatggcaaacgttgctaggtattctgtcaatactggtcatccatacttcgttaaccagttattctcatccgttgatccatatggtttagttggtcaatggttaaccgacgcattgaacccatccgtttacacctttgaagtcgctcctgtctttaccttgatggaggaagaggttttgcgtgaaatgcgtaagattgtcggttggcctgagggcgaaggtgatggtatcttctgcccaggtggttctatcgccaacggttacgctatttcctgtgctagacatcacttttacccagaggtcaagtataagggtgttcatgctgttcctaagttagttttgtttacctccgaactcgcgcactacagtaccaaaaagatggctgctttcatgggtattggttccgataactgtgttaacattaagaccgatgatgttggtaagatgaacatcgttgatttagaaatgaagatcaagattgctattgacaacaagtgtaccccattcatggttacagctacctctggtactaccgttttcggtgctttcgacccattagtcgcaatctccgatctttgcaagaagtacaatctgtggttgcatgtcgatgctgcttggggtggtggtgctctcatgtctaagaagcacagacaccttttgaacggtatcgaattagccgactctgttacttggaaccctcataagcttttagccgctccacaacaatgttccacctttttgaccagacacaagaaagttctatccgaaggtcattcatccaatgcaaagtacttattccaaaaagacaagttttacgacacatcctacgacactggtgataaacatattcaatgtggtagaagagcagatgttcttaagttctggttcatgtggaaggctaagggtactgaaggtttcgagaaacacgttgacaagctttttgataacgcaaagtacttcttagaccacattaagcagagagaaggtttccaattggttattgcagaaccacaatgcaccaatatcatgttctggtacatcccaaagtgcctgagaggttgtgaaaatgatgcagactactatgagagattacataaagttgcgcctaagatcaaggaaagaatgatcaaggaaggttcaatgatggtcacctaccaaccacaaggtgatcttgttaacttctttagaattgtttttcagaactctgcattggaccataaggacatggtttacttcgcaaatgaattcgaaagattgggctccgatatgatcgtttaattaattaatttattttactagtttatttttgctcctgagaataggattacaaacacttaaagtctttaattacaactatatataatattctgttggttttcttgaattggttcgctgcgattcatgcctcccattcaccaaaggtggagtgggaaataacggttttactgcggtaattagcagaggcaagaacaggatacactttttgatgataaatctgtattatagtcgagcctatttaggaaatcaaattttcttgtgtttacttttcaaataaataatgttcgaaaatttttactttactccttcatttaactataccagacgttatatcatcaacaccttctgaccatatacagctcaagatgtttaagagtctgttaaattttttcaatccatttcatggcctgcagggataacttcgtataatgtatgctatacgaagttatgctgcaacggcaacatcaatgtccacgtttacacacctacatttatatctatatttatatttatatttatttatttatgctacttagcttctatagttagttaatgcactcacgatattcaaaattgacacccttcaactactccctactattgtctactactgtctactactcctctttactatagctgctcccaataggctccaccaataggctctgtcaatacattttgcgccgccacctttcaggttgtgtcactcctgaaggaccatattgggtaatcgtgcaatttctggaagagagtgccgcgagaagtgaggcccccactgtaaatcctcgagggggcatggagtatggggcatgaaggatggaggatggggggggggggggaaaataggtagcgaaaggacccgctatcaccccacccggagaactcgttgccgggaagtcatatttcgacactccggggagtctataaaaggcgggttttgtcttttgccagttgatgttgctgagaggacttgtttgccgtttcttccgatttaacagtatagaatcaaccactgttaattatacacgttatactaacacaacaaaaacaaaaacaacgacaacaacaatctagatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggc

SEQ ID NO: 39 - DNA, 5656 bp, Artificial Sequence
  A synthetic oligonucleotide sequence
tcgagggggcatggagtatggggcatgaaggatggaggatggggggggggggggaaaataggtagcgaaaggacccgctatcaccccacccggagaactcgttgccgggaagtcatatttcgacactccggggagtctataaaaggcgggttttgtcttttgccagttgatgttgctgagaggacttgtttgccgtttcttccgatttaacagtatagaatcaaccactgttaattatacacgttatactaacacaacaaaaacaaaaacaacgacaacaacaatctagatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagaaattcggatccggtagatacattgatgctatcaatccagagaactggaaagattgtgtagccttgaaaaacggtgaaacttacgggtccaagattgtctacagattttcctgatttgccagcttactatccttcttgaaaatatgcactctatatcttttagttcttaattgcaacacatagatttgctgtataacgaattttatgctattttttaaatttggagttcagtgataaaagtgtcacagcgaatttcctcacatgtagggaccgaattgtttacaagttctctgtaccataacttcgtataatgtatgctatacgaagttattaagttaaacagcaatttgaggaaggaataggagaaggagaagcaatttctaggaaagagcaaggtgtgcaacagcatgctctgaatgatattttcagcaatagttcagttgaagaacctgttggcgtatctacatcacttcctacaaacaacaccacgaattgcgtccgtggtgacgcaactacgaatggcattgtcaatgccaatgccagtgcacatacacgtgcaagtcccaccggttccctgcccggctatggtagagacaagaaggacgataccggcatcgacatcaacagtttcaacagcaatgcgtttggcgtcgacgcgtcgatggggctgccgtatttggatttggacgggctagatttcgatatggatatggatatggatatggatatggagatgaatttgaatttagatttgggtcttgatttggggttggaattaaaaggggataacaatgagggttttcctgttgatttaaacaatggacgtgggaggtgattgatttaacctgatccaaaaggggtatgtctattttttagagtgtgtctttgtgtcaaattatggtagaatgtgtaaagtagtataaactttcctctcaaatgacgaggtttaaaacaccccccgggtgagccgagccgagaatggggcaattgttcaatgtgaaatagaagtatcgagtgagaaacttgggtgttggccagccaagggggaaggaaaatggcgcgaatgctcaggtgagattgttttggaattgggtgaagcgaggaaatgagcgacccggaggttgtgactttagtggcggaggaggacggaggaaaagccaagagggaagtgtatataaggggagcaatttgccaccaggatagaattggatgagttataattctactgtatttattgtataatttatttctccttttatatcaaacacattacaaaacacacaaaacacacaaacaaacacatctagataaaatgagggttgactccaaaatcatcgtcaaaaaggaaacccaagatgaaaacttgtaccaatctttggctgaaagatccaagcatgaagagtttttgagaaaagccgttgatttgttggttgaaagagttgtttttggtagatccactagatcctccaaggtcgttgaatgggcagctccagatgaaatcaaaaaggccatcgatcttaagcctaggttaggcccagcttcccatgatgaattgttagcctttatggcaaatgttgcaagatattccgtcaatactggtcacccatacttcgttaatcaattgttttcatctgttgatccatacggtctagttggtcaatggttgaccgatgccttaaacccttctgtctatacctttgaagttgctccagtctttacattgatggaggaagaggttttgagagaaatgaggaaaatcgttggttggccagaaggtgagggtgatggcatcttttgtccaggtggttctatcgccaacggttacgctatttcttgtgctcgtcatcacttctacccagaagttaagtacaaaggtgttcatgctgtccctaagttagtcttgtttacctctgaattagctcactattctacaaagaaaatggctgcattcatgggtattggttccgacaattgtgtcaatatcaaaacagacgacgttggtaagatgaacattgttgacctagaaatgaagattaagatcgcaattgacaacaagtgtacccctttcatggttacagctacatcaggtactactgttttcggtgcttttgacccacttgtcgcaatctccgatttgtgtaagaagtacaacttatggctacatgtcgatgcggcatggggtggtggtgcgttaatgtcaaagaagcatagacatcttctaaacggtatcgaattagcagactccgttacctggaatccacacaaattgttagctgctcctcaacaatgttccacattccttaccagacacaagaaggtcctgtctgaaggtcattcttccaacgccaaatacttgtttcagaaggacaagttttacgacacttcttatgacactggtgacaaacatattcagtgtggtagaagagcagatgttttgaagttctggtttatgtggaaggcaaagggtacggagggtttcgagaagcacgttgataagttgtttgacaatgctaagtacttcctagatcacattaagcaaagggaaggtttccaattggtcattgcagaacctcagtgtaccaacatcatgttctggtatatcccaaagtgtctaagaggttgtgaaaatgatgcagactactatgaaagattgcataaagtcgctccaaagatcaaagaaagaatgatcaaagaaggttctatgatggtcacctaccaaccacaaggtgatttggtcaatttctttagaatcgttttccagaattctgctttagatcataaggatatggtctactttgcaaacgaattcgaaagattaggttccgacatgatcgtttaattaacatctgaatgtaaaatgaacattaaaatgaattactaaactttacgtctactttacaatctataaactttgtttaatcatataacgaaatacactaatacacaatcctgtacgtatgtaatacttttatccatcaaggattgagaaaaaaaagtaatgattccctgggccattaaaacttagacccccaagcttggataggtcactctctattttcgtttctcccttccctgatagaagggtgatatgtaattaagaataatatataattttataataaaagcggccgccaacttggttcaaaccaagaaagccgtcgtgtactttgcgtattttcaacttcttcccttcaactccaaccttataacccaatccgacagttagcatacagtagaaaatccataagttaattgcaaaaaccacataattcccaatgactggcacaatttgcatgaacggcgcaaaaccaacacctatccttcttgacttgtggacaacttctccggcatcggctccagtttctggtttaggcgacactttcaaggtacccttgttgactaggttctcagcaacatactcctcctcgataactttgaccgtctttgtgccaaacacattgttcaatagtttggacgctcgcataccgaagacagactcgtccatccaatagcatgtactccgaatgctctcaatcaacggtatcaaatcgcttggtgtctttggaggcggtgttctaggtttcatcttcgccttcccgtttcctgtttcttggacttcaaaatacgggtcatatgcatcttcattggtgaacctgaacagtttgggccgagtagtgccaaacaccttcaacagtataatcttcactccttggccgattagaaacatgcttatatgtttgccggtattcttaaagtgtatactggagcccttttaatcacattttttttacttgtcagtctccatacggagtttaatgtccttatatcgatcttcatcagctccaacgggacaggaatcaataaccttccttgcctgtcccaaaaagaatgaattttttttcaaaagctttacgatgcataccacaaaaggaagattattcccacatgttccagaagtgtgcggagatacaaagggttcatgaaaacgtgaatcttctaaaaacttagcacaacaataaaaatctacaatgttacagtaagtattattttctttttgtcgacacactccaacggttagatttccaagtattcaatccaatgtattacttgtcagacagccatccactcccatcttagaacatcacttccgaaccgcggagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccag

SEQ ID NO: 40 - DNA, 3235 bp, Artificial Sequence
  A synthetic oligonucleotide sequence
cgcccagcagatatcaatgctttacttggggaaatccatcaaggtgcaaggttgaagaaggtcgacgacagcgagaaacacattgctgacggtgccgttgttggcagagtattgtaattgcaacatgttcctgtttagtctagcatatatatacttacttataaagctaaaatattgcgttttgttctagttttaactgccgtctctttcttgccgagccccctttattttcttcctttagwgaggcaataawatttcgataagcgcaatcccgaattttttctgcggcgagagagatgcgggactgggcttccgaagaacgtaaccaaactgattttatccaatataaaagagagggtgccacagctgtttgcgggagaaggtactcttatttacacggtagtcttctctcacatttcagatataatacaagcaatattcaacagcagacaggcctgcggccgcggatcaattcgcccttacatatggataacttcgtataatgtatgctatacgaagttatgctgcaacggcaacatcaatgtccacgtttacacacctacatttatatctatatttatatttatatttatttatttatgctacttagcttctatagttagttaatgcactcacgatattcaaaattgacacccttcaactactccctactattgtctactactgtctactactcctctttactatagctgctcccaataggctccaccaataggctctgtcaatacattttgcgccgccacctttcaggttgtgtcactcctgaaggaccatattgggtaatcgtgcaatttctggaagagagtgccgcgagaagtgaggcccccactgtaaatcctcgagggggcatggagtatggggcatggaggatggaggatggggggggggggggaaaataggtagcgaaaggacccgctatcaccccacccggagaactcgttgccgggaagtcatatttcgacactccggggagtctataaaaggcgggttttgtcttttgccagttgatgttgctgagaggacttgtttgccgtttcttccgatttaacagtatagaatcaaccactgttaattatacacgttatactaacacaacaaaaacaaaaacaacgacaacaacaacaacataaaatgaagtttacacaaattgcacaagctttagcactagcaggatctgcaactgctgtttctccgagttacaatggtcttggtctcaccccacagatgggttgggacagctggaatacgtttgcctgcgatgtcagtgaacagctacttctagacactgctgatagagtttctgacttggggctaaaggatatgggttacaagtatgtcatcctagatgactgttggtctagcggcagggattccgacggtttcctcgttgcagacaagcacaaatttcccaacggtatgggccatgttgcagaccacctgcataataacagctttcttttcggtatgtattcgtctgctggtgagtacacctgtgctgggtaccctgggtctctggggcgtgaggaagaagatgctcaattctttgcaaataaccgcgttgactacttgaagtatgataattgttacaataaaggtcaatttggtacaccagacgtttcttaccaccgttacaaggccatgtcagatgctttgaataaaactggtaggcctattttctattctctatgtaactggggtcaggatttgacattttactggggctctggtatcgccaattcttggagaatgagcggagatattactgctgagttcacccgcccagatagcagatgtccctgtgacggtgacgaatatgattgcaagtacgccggtttccattgttctattatgaatattcttaacaaggcagctccaatggggcaaaatgcaggtgttggtggttggaacgatctggacaatctagaggtcggagtcggtaacttgactgacgatgaggaaaaggcccatttctctatgtgggcaatggtaaagtccccacttatcattggtgccgacgtgaatcacttaaaggcatcttcgtactcgatctacagtcaagcctctgtcatcgcaattaatcaagatccaaagggtattccagccacaagagtctggagatattatgtttcagacaccgatgaatatggacaaggtgaaattcaaatgtggagtggtccgcttgacaatggtgaccaagtggttgctttattgaatggaggaagcgtagcaagaccaatgaacacgaccttggaagagattttctttgacagcaatttgggttcaaaggaactgacatcgacttgggatatttacgacttatgggccaacagagttgacaactctacggcgtctgctatccttgaacagaataaggcagccaccggtattctctacaatgctacagagcagtcttataaagacggtttgtctaagaatgatacaagactgtttggccagaaaattggtagtctttctccaaatgctatacttaacacaactgttccagctcatggtatcgccttctataggttgagaccctcggcttaagctcaatgttgagcaaagcaggacgagaaaaaaaaaaataatgattgttaagaagttcatgaaaaaaaaaaggaaaaatactcaaatacttataacagagtgattaaataataaacggcagtataccctatcaggtattgagatagttttatttttgtaggtatataatctgaagcctttgaactattttctcgtatatatcatggagtatacattgcattagcaacattgcatactagttcataacttcgtataatgtatgctatacgaagttattaattaagccgaaaaggtctatgagggaaccgagccattgtatggtaccgatattgcagaattgattctatttgcagtttctagacctcaaaacactgttattgcagaaacacttgtttttgctagtaaccaagcttctgcttaccatattttcagaggatcattagataaatagatttgatataaacgcttctataataataaataayatcaacaagtgactaaccccaagtatccaattttagayctaatgcctgaaagtgccttgcacgatttaggattcggcaccagaaagtttgtgccgtgctcgcttattggacttgtatagtgataaggcaaaaaaaaaaaacttgaaagtacttgcgtaactcagaggttgccttttcgggcc

SEQ ID NO: 41 - DNA, 3136 bp, Artificial Sequence
  A synthetic oligonucleotide sequence
cgcccagcagatatcaatgctttacttggggaaatccatcaaggtgcaaggttgaagaaggtcgacgacagcgagaaacacattgctgacggtgccgttgttggcagagtattgtaattgcaacatgttcctgtttagtctagcatatatatacttacttataaagctaaaatattgcgttttgttctagttttaactgccgtctctttcttgccgagccccctttattttcttcctttagagaggcaataaaatttcgataagcgcaatcccgaattttttctgcggcgagagagatgcgggactgggcttccgaagaacgtaaccaaactgattttatccaatataaaagagagggtgccacagctgtttgcgggagaaggtactcttatttacacggtagtcttctctcacatttcagatataatacaagcaatattcaacagcagacgcggccgcgttgttgatgctgcgcacctgtggttgcccaacatggttgtatatcgtgtaaccacaccaacacatgtgcagcacatgtgtttaaaagagtgtcatggaggtggatcatgatggaagtggactttaccacttgggaactgtctccactcccgggaagaaaagacccggcgtatcacgcggttgcctcaatggggcaatttggaaggagaaatatagggaaaatcacgtcgctctcggacggggaagagttccagactatgaggggggggggtggtatataaagacaggagatgtccacccccagagagaggaagaagttggaactttagaagagagagataactttccccagtgtccatcaatacacaaccaaacacaaactctatatttacacatataaccccctccaaccaaaaggctagcatatatcgatcgtaaatctaactaatgcttttactaaatatctagtacaatttttacagtccctacgtttataaatgaatttaatgaaaaaaaaatattttgtaacgatgtgtttattaagttgcgctcttccgataatcccggactttggttaatttctcaatgggtttttttttcaaaaccattgttgtagtgtaacagactttaacaaaaggacatcactctacagggcagctttaaaatccctcagtgtaattgttcttcattcataacgtggcagtcaaggactcgaggagtccatcggttcctgtcagatgggatactcttgacgtggaaaattcaaacagaaaaaaaaccccaataatgaaaaataacactacgttatatccgtggtatcctctatcgtatcgtatcgtagcgtatcgtagcgtaccgtatcacagtatagtctaatattccgtatcttattgtatcctatcctattcgatcctattgtatttcagtgcaccattttaatttctattgctataatgtccttattagttgccactgtgaggtgaccaatggacgagggcgagccgttcagaagccgcgaagggtgttcttcccatgaatttcttaaggagggcggctcagctccgagagtgaggcgagacgtctcggtcagcgtatcccccttcctcggcttttacaaatgatgcgctcttaatagtgtgtcgttatccttttggcattgacgggggagggaaattgattgagcgcatccatatttttgcggactgctgaggacaatggtggtttttccgggtggcgtgggctacaaatgatacgatggtttttttcttttcggagaaggcgtataaaaaggacacggagaacccatttattctaaaaacagttgagcttctttaattattttttgatataatattctattattatatattttcttcccaataaaacaaaataaaacaaaacacagcaaaacacaaaaattctagactatcttaattaacatctgaatgtaaaatgaacattaaaatgaattactaaactttacgtctactttacaatctataaactttgtttaatcatataacgaaatacactaatacacaatcctgtacgtatgtaatacttttatccatcaaggattgagaaaaaaaagtaatgattccctgggccattaaaacttagacccccaagcttggataggtcactctctattttcgtttctcccttccctgatagaagggtgatatgtaattaagaataatatataattttataataaaagaattcgcccttacatatgataacttcgtataatgtatgctatacgaagttatcatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaactttttttttttcttggtgcacggacatgtttttaaaggaagtactctataccagttattcttcacaaatttaattgctggagaatagatcttcaacgctttaataaagtagtttgtttgtcaaggatggcgtcatacaaagaaagatcagaatcacacacttcccctgttgctaggagacttttctccatcatggaggaaaagaagtctaacctttgtgcatcattggatattactgaaactgaaaagcttctctctattttggacactattggtccttacatctgtctagttaaaacacacatcgatattgtttctgattttacgtatgaaggaactgtgttgcctttgaaggagcttgccaagaaacataattttatgatttttgaagatagaaaatttgctgatattggtaacactgttaaaaatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagggcc

SEQ ID NO: 42 - DNA, 5700 bp, Artificial Sequence
  A synthetic oligonucleotide sequence
ccacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactggatcattatgactccaggggttggtttagatgacaaaggtgatgcacttggtcaacaatatagaactgttgatgaagttgtaaagactggaacggatatcataattgttggtagaggtttgtacggtcaaggaagagatcctatagagcaagctaaaagataccaacaagctggttggaatgcttatttaaacagatttaaatgattcttacacaaagatttgatacatgtacactagtttaaataagcatgaaaagaattacacaagcaaaaaaaaaaaaataaatgaggtactttacgttcacctacaaccaaaaaaactagatagagtaaaatcttaagatttagaaaaagttgtttaacaaaggctttagtatgtgaatttttaatgtagcaaagcgataactaataaacataaacaaaagtatggttttctttatcagtcaaatcattatcgattgattgttccgcgtatctgcagataacttcgtataatgtatgctatacgaagttatagatcgcggccgctaacctgatccaaaaggggtatgtctattttttagagtgtgtctttgtgtcaaattatggtagaatgtgtaaagtagtataaactttcctctcaaatgacgaggtttaaaacaccccccgggtgagccgagccgagaatggggcaattgttcaatgtgaaatagaagtatcgagtgagaaacttgggtgttggccagccaagggggaaggaaaatggcgcgaatgctcaggtgagattgttttggaattgggtgaagcgaggaaatgagcgacccggaggttgtgactttagtggcggaggaggacggaggaaaagccaagagggaagtgtatataaggggagcaatttgccaccaggatagaattggatgagttataattctactgtatttattgtataatttatttctccttttatatcaaacacattacaaaacacacaaaacacacaaacaaacacaattacaaaaagctagcatgactagaacttttgaattgggtgaattgattcgttctgataacttcattgatggtgcttggactccagcacaagacaacttgaggttcgctgtcactaacccagcttctggagagataattgctgaggtcgctgactcttctccagctgatgcaagagctgctactgatgcagctgcaagagctttgccagcttggagggctagattgccaaaagagagagctgcagtcttgcatcgttggcacgctttgataatggctaacttggatgcattgggtgctttaatatctttggagcaaggtaaacctttggctgagggtaagggtgaggtcgcttatggtgcttcttacgtcgcatggttcgcagaggaagcaacaagaatttacggtgatttgattcctcagcaacagaggggtaagaggatgactgctgtcaaagagccagtcggagtcgttgctgctattacaccatggaattggccattggcaatgattgcaagaaagatagcacccgctttggcagctggttgtactgttgtcgctaagccagctgaggacactccattgactgcttcagctttggtcttgttggctcacgaagctggtgttccacccggtgttttgaacttgattactgcatcccgtgatcatgctgtcgcagcagtcgctgagtggttgcatgacgctagagttagaaaaattacttttactggatcaactccagtcggtaagtacttggctagggaatctgctgaaactttaaagaagttatctttggagttgggtggtaatgctccatttattgtttttgacgatgctgacttggaggctgcagtcgctggtttgatggctgctaagtttcgtaacggtggtcagacttgtgtctgtccaaatcgtgtctacgtccaagctggtgtctacgagaggtttggtgctttgttggctgaaagggttggtgctttgaaggttgctccagcaactgatccagctgctcaaattggtccaatgattaattctagggcattggacaagattgctaggcacgtcgatgacgctgttgcacatggtgctagagtcttgactggtggtaagaggttggcagagttgggtccacactactacgctccaactgttttggctgacgcaacagcagcaatgcagttgaactcagaggaaactttcggtccaatagtcccattgtttcgtttcgaggacgaggctgaagcagttaacgctgctaacgacactccattcggtttagcagcttatttttattctgaaggtgtcaaaagaattgatagggtcgctagggctttggaagctggtattgttggtataaatgaaggtgcagtcgcttcagaagctgctccattcggtggtgttaaggaatctggttacggtagagaaggttctaaatatggtttggatgattatttgtctattaaatatttatgtcaaggtaatttagaataacgatcgtaagcggcgaatctctggctcatgggggatatcctctttgtttggcttttttttcccattctctgttttgattatctaatgactcattgggaggattttctcacttcaagcttttttttcttgcactctttcataactccagctctctctaactgaggctacaatgccttttaacgaacttatgagacgtttctaaattatataggtatatgccaatatataattacacataaaaataaatataaataaaatataaaaataaaaataaacatcgaaaaagaagatgtgaaattgcgaagactagaaagcacaaacgagcggtctatatcggcgactcgaggctctacaagcctcatatgggttcaatgggtctgcaatgaccgcatacggacttggacaattaccttctattgaatttctgagaagagatacatctgaccagcaatgtaagcagacaatcccaattctgtaaacaacctctttgtccataattccccatcagaagagtgaaaaatgccctcaaaacgcatgcgccactcccacctctcagctgcactgcgccacctctgagggtcctttcaggggtcgactaccccggacacctcgcagaggagcgagatcacgtacttttaaaatggcagagacgcgcagtttcttgaagaaaggataaaaatgaaatggtgcggaaatgcgaaaatgatgaaaaattttcttggtggcgaggaaattgagtgcaataattggcacgaggttgttgccacccgagtgtgagtatatatcctagtttctgcacttttcttcttcttttccttgcgttttcttttcaactttttttactttttccttcaacagacaaatctaacttatatatcacatctagaatgactagaacttttgaattgggtgaattgattcgttctgataacttcattgatggtgcttggactccagcacaagacaacttgaggttcgctgtcactaacccagcttctggagagataattgctgaggtcgctgactcttctccagctgatgcaagagctgctactgatgcagctgcaagagctttgccagcttggagggctagattgccaaaagagagagctgcagtcttgcatcgttggcacgctttgataatggctaacttggatgcattgggtgctttaatatctttggagcaaggtaaacctttggctgagggtaagggtgaggtcgcttatggtgcttcttacgtcgcatggttcgcagaggaagcaacaagaatttacggtgatttgattcctcagcaacagaggggtaagaggatgactgctgtcaaagagccagtcggagtcgttgctgctattacaccatggaattggccattggcaatgattgcaagaaagatagcacccgctttggcagctggttgtactgttgtcgctaagccagctgaggacactccattgactgcttcagctttggtcttgttggctcacgaagctggtgttccacccggtgttttgaacttgattactgcatcccgtgatcatgctgtcgcagcagtcgctgagtggttgcatgacgctagagttagaaaaattacttttactggatcaactccagtcggtaagtacttggctagggaatctgctgaaactttaaagaagttatctttggagttgggtggtaatgctccatttattgtttttgacgatgctgacttggaggctgcagtcgctggtttgatggctgctaagtttcgtaacggtggtcagacttgtgtctgtccaaatcgtgtctacgtccaagctggtgtctacgagaggtttggtgctttgttggctgaaagggttggtgctttgaaggttgctccagcaactgatccagctgctcaaattggtccaatgattaattctagggcattggacaagattgctaggcacgtcgatgacgctgttgcacatggtgctagagtcttgactggtggtaagaggttggcagagttgggtccacactactacgctccaactgttttggctgacgcaacagcagcaatgcagttgaactcagaggaaactttcggtccaatagtcccattgtttcgtttcgaggacgaggctgaagcagttaacgctgctaacgacactccattcggtttagcagcttatttttattctgaaggtgtcaaaagaattgatagggtcgctagggctttggaagctggtattgttggtataaatgaaggtgcagtcgcttcagaagctgctccattcggtggtgttaaggaatctggttacggtagagaaggttctaaatatggtttggatgattatttgtctattaaatatttatgtcaaggtaatttagaataattaattaagtatagccatatagtttaattcctttatactttttataactatttcttacactaattattattatcaattatttattgtagaacttgactcttgcgtcgatcaccatgacagggctatcttaacaaggggtaatttttgttgatggagtcaagtagcattccgacgggaagtgtcgatgcctctgaacgaaatcttccgattagctctgcaaagaagtggaaattgtcagcgcagaattcgccgaaaaggtctatgagggaaccgagccattgtatggtaccgatattgcagaattgattctatttgcagtttctacacctcaaaacactgttattgcagaaacacttgtttttgctagtaaccaagcttctgcttaccatattttcagaggatcattagataaatagatttgatataaacgcttctataataataaataacatcaacaagtgactaaccccaagtatccaattttagacctaatgcctgaaagtgccttgcacgatttaggattcggcaccagaaagtttgtgccgtgctcgcttattggacttgtatagtgataaggcaaaaaaaaaaaacttgaaagtacttgcgtaactcagaggttgccttttcgggcc

SEQ ID NO: 43 - DNA, 6071 bp, Artificial Sequence
  A synthetic oligonucleotide sequence
cgcccagcagatatcaatgctttacttggggaaatccatcaaggtgcaaggttgaagaaggtcgacgacagcgagaaacacattgctgacggtgccgttgttggcagagtattgtaattgcaacatgttcctgtttagtctagcatatatatacttacttataaagctaaaatattgcgttttgttctagttttaactgccgtctctttcttgccgagccccctttattttcttcctttagagaggcaataaaatttcgataagcgcaatcccgaattttttctgcggcgagagagatgcgggactgggcttccgaagaacgtaaccaaactgattttatccaatataaaagagagggtgccacagctgtttgcgggagaaggtactcttatttacacggtagtcttctctcacatttcagatataatacaagcaatattcaacagcagacgcggccgcgttgttgatgctgcgcacctgtggttgcccaacatggttgtatatcgtgtaaccacaccaacacatgtgcagcacatgtgtttaaaagagtgtcatggaggtggatcatgatggaagtggactttaccacttgggaactgtctccactcccgggaagaaaagacccggcgtatcacgcggttgcctcaatggggcaatttggaaggagaaatatagggaaaatcacgtcgctctcggacggggaagagttccagactatgaggggggggggtggtatataaagacaggagatgtccacccccagagagaggaagaagttggaactttagaagagagagataactttccccagtgtccatcaatacacaaccaaacacaaactctatatttacacatataaccccctccaaccaaaaggctagcatgactagaacttttgaattgggtgaattgattcgttctgataacttcattgatggtgcttggactccagcacaagacaacttgaggttcgctgtcactaacccagcttctggagagataattgctgaggtcgctgactcttctccagctgatgcaagagctgctactgatgcagctgcaagagctttgccagcttggagggctagattgccaaaagagagagctgcagtcttgcatcgttggcacgctttgataatggctaacttggatgcattgggtgctttaatatctttggagcaaggtaaacctttggctgagggtaagggtgaggtcgcttatggtgcttcttacgtcgcatggttcgcagaggaagcaacaagaatttacggtgatttgattcctcagcaacagaggggtaagaggatgactgctgtcaaagagccagtcggagtcgttgctgctattacaccatggaattggccattggcaatgattgcaagaaagatagcacccgctttggcagctggttgtactgttgtcgctaagccagctgaggacactccattgactgcttcagctttggtcttgttggctcacgaagctggtgttccacccggtgttttgaacttgattactgcatcccgtgatcatgctgtcgcagcagtcgctgagtggttgcatgacgctagagttagaaaaattacttttactggatcaactccagtcggtaagtacttggctagggaatctgctgaaactttaaagaagttatctttggagttgggtggtaatgctccatttattgtttttgacgatgctgacttggaggctgcagtcgctggtttgatggctgctaagtttcgtaacggtggtcagacttgtgtctgtccaaatcgtgtctacgtccaagctggtgtctacgagaggtttggtgctttgttggctgaaagggttggtgctttgaaggttgctccagcaactgatccagctgctcaaattggtccaatgattaattctagggcattggacaagattgctaggcacgtcgatgacgctgttgcacatggtgctagagtcttgactggtggtaagaggttggcagagttgggtccacactactacgctccaactgttttggctgacgcaacagcagcaatgcagttgaactcagaggaaactttcggtccaatagtcccattgtttcgtttcgaggacgaggctgaagcagttaacgctgctaacgacactccattcggtttagcagcttatttttattctgaaggtgtcaaaagaattgatagggtcgctagggctttggaagctggtattgttggtataaatgaaggtgcagtcgcttcagaagctgctccattcggtggtgttaaggaatctggttacggtagagaaggttctaaatatggtttggatgattatttgtctattaaatatttatgtcaaggtaatttagaataacgatcgtaaatctaactaatgcttttactaaatatctagtacaatttttacagtccctacgtttataaatgaatttaatgaaaaaaaaatattttgtaacgatgtgtttattaagttgcgctcttccgataatcccggactttggttaatttctcaatgggtttttttttcaaaaccattgttgtagtgtaacagactttaacaaaaggacatcactctacagggcagctttaaaatccctcagtgtaattgttcttcattcataacgtggcagtcaaggactcgaggagtccatcggttcctgtcagatgggatactcttgacgtggaaaattcaaacagaaaaaaaaccccaataatgaaaaataacactacgttatatccgtggtatcctctatcgtatcgtatcgtagcgtatcgtagcgtaccgtatcacagtatagtctaatattccgtatcttattgtatcctatcctattcgatcctattgtatttcagtgcaccattttaatttctattgctataatgtccttattagttgccactgtgaggtgaccaatggacgagggcgagccgttcagaagccgcgaagggtgttcttcccatgaatttcttaaggagggcggctcagctccgagagtgaggcgagacgtctcggtcagcgtatcccccttcctcggcttttacaaatgatgcgctcttaatagtgtgtcgttatccttttggcattgacgggggagggaaattgattgagcgcatccatatttttgcggactgctgaggacaatggtggtttttccgggtggcgtgggctacaaatgatacgatggtttttttcttttcggagaaggcgtataaaaaggacacggagaacccatttattctaaaaacagttgagcttctttaattattttttgatataatattctattattatatattttcttcccaataaaacaaaataaaacaaaacacagcaaaacacaaaaattctagaatgactagaacttttgaattgggtgaattgattcgttctgataacttcattgatggtgcttggactccagcacaagacaacttgaggttcgctgtcactaacccagcttctggagagataattgctgaggtcgctgactcttctccagctgatgcaagagctgctactgatgcagctgcaagagctttgccagcttggagggctagattgccaaaagagagagctgcagtcttgcatcgttggcacgctttgataatggctaacttggatgcattgggtgctttaatatctttggagcaaggtaaacctttggctgagggtaagggtgaggtcgcttatggtgcttcttacgtcgcatggttcgcagaggaagcaacaagaatttacggtgatttgattcctcagcaacagaggggtaagaggatgactgctgtcaaagagccagtcggagtcgttgctgctattacaccatggaattggccattggcaatgattgcaagaaagatagcacccgctttggcagctggttgtactgttgtcgctaagccagctgaggacactccattgactgcttcagctttggtcttgttggctcacgaagctggtgttccacccggtgttttgaacttgattactgcatcccgtgatcatgctgtcgcagcagtcgctgagtggttgcatgacgctagagttagaaaaattacttttactggatcaactccagtcggtaagtacttggctagggaatctgctgaaactttaaagaagttatctttggagttgggtggtaatgctccatttattgtttttgacgatgctgacttggaggctgcagtcgctggtttgatggctgctaagtttcgtaacggtggtcagacttgtgtctgtccaaatcgtgtctacgtccaagctggtgtctacgagaggtttggtgctttgttggctgaaagggttggtgctttgaaggttgctccagcaactgatccagctgctcaaattggtccaatgattaattctagggcattggacaagattgctaggcacgtcgatgacgctgttgcacatggtgctagagtcttgactggtggtaagaggttggcagagttgggtccacactactacgctccaactgttttggctgacgcaacagcagcaatgcagttgaactcagaggaaactttcggtccaatagtcccattgtttcgtttcgaggacgaggctgaagcagttaacgctgctaacgacactccattcggtttagcagcttatttttattctgaaggtgtcaaaagaattgatagggtcgctagggctttggaagctggtattgttggtataaatgaaggtgcagtcgcttcagaagctgctccattcggtggtgttaaggaatctggttacggtagagaaggttctaaatatggtttggatgattatttgtctattaaatatttatgtcaaggtaatttagaataattaattaacatctgaatgtaaaatgaacattaaaatgaattactaaactttacgtctactttacaatctataaactttgtttaatcatataacgaaatacactaatacacaatcctgtacgtatgtaatacttttatccatcaaggattgagaaaaaaaagtaatgattccctgggccattaaaacttagacccccaagcttggataggtcactctctattttcgtttctcccttccctgatagaagggtgatatgtaattaagaataatatataattttataataaaagaattcgcccttacatatgataacttcgtataatgtatgctatacgaagttatcatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaactttttttttttcttggtgcacggacatgtttttaaaggaagtactctataccagttattcttcacaaatttaattgctggagaatagatcttcaacgctttaataaagtagtttgtttgtcaaggatggcgtcatacaaagaaagatcagaatcacacacttcccctgttgctaggagacttttctccatcatggaggaaaagaagtctaacctttgtgcatcattggatattactgaaactgaaaagcttctctctattttggacactattggtccttacatctgtctagttaaaacacacatcgatattgtttctgattttacgtatgaaggaactgtgttgcctttgaaggagcttgccaagaaacataattttatgatttttgaagatagaaaatttgctgatattggtaacactgttaaaaatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagggcc

SEQ ID NO: 44 - DNA, 5676 bp, Artificial Sequence
  A synthetic oligonucleotide sequence
ccacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactggatcattatgactccaggggttggtttagatgacaaaggtgatgcacttggtcaacaatatagaactgttgatgaagttgtaaagactggaacggatatcataattgttggtagaggtttgtacggtcaaggaagagatcctatagagcaagctaaaagataccaacaagctggttggaatgcttatttaaacagatttaaatgattcttacacaaagatttgatacatgtacactagtttaaataagcatgaaaagaattacacaagcaaaaaaaaaaaaataaatgaggtactttacgttcacctacaaccaaaaaaactagatagagtaaaatcttaagatttagaaaaagttgtttaacaaaggctttagtatgtgaatttttaatgtagcaaagcgataactaataaacataaacaaaagtatggttttctttatcagtcaaatcattatcgattgattgttccgcgtatctgcagataacttcgtataatgtatgctatacgaagttatagatcgcggccgctaacctgatccaaaaggggtatgtctattttttagagtgtgtctttgtgtcaaattatggtagaatgtgtaaagtagtataaactttcctctcaaatgacgaggtttaaaacaccccccgggtgagccgagccgagaatggggcaattgttcaatgtgaaatagaagtatcgagtgagaaacttgggtgttggccagccaagggggaaggaaaatggcgcgaatgctcaggtgagattgttttggaattgggtgaagcgaggaaatgagcgacccggaggttgtgactttagtggcggaggaggacggaggaaaagccaagagggaagtgtatataaggggagcaatttgccaccaggatagaattggatgagttataattctactgtatttattgtataatttatttctccttttatatcaaacacattacaaaacacacaaaacacacaaacaaacacaattacaaaaagctagcatgcacatgtctgagactcctattaacactcaattattgattgctggtcaatggcaagacgctgctgacggtagaactttgccaattttagatccatctaatggtgaattgattggtaaagtcgcacacgcttctgtcgcagatttggataagacagttgctggtgcagctgctggttacgacgcttggagaaacttgactgcatgggatcgttacactattatgaggagggctgctgagttgttaagggagagagctgatgctattggtagaattatgtcaagagagcaaggtaagccattggctcaagctgttattgaaattaatgctgctgctggtgtcattgagtacttcggagaggaaggtagaaggttgaacgatgagttggtcccagctcgtgctccttttgtcgagcagagagtcttgcatcgtccaataggtgtcgttgcagctttcactccatggaactggccaattaatcaaattgctagaaagttgggagctgctttaggtgctggttgtggtgtcatagtcaaggctccagaagacacacccgcttctcccgctgagttgattagatgtttctgtgatgctggtattccagctggtgcagtctctttggtttacggtactcccgctgaaatttcagaatatttgattgctcatccagttgttagaaaaatttctttcactggttctacacccgttggtaagcacttggctgcattggctggtgcacaaatgaagccagtcacaatggaattgggtggtcacggtcccgttattgtctgcagagatgctgacttagatcaagctgctgaaagggttgcagctgctaagtttcgtaacgctggtcaagtttgcactgcaccaactagatttttattgcataaagatattgcttctgcttttattgaaaagtttaaagctcaaatgtcagctttaaagataggttgcggtttggatgagggtgtcacaatgggtccattaataaacgaaagaagagttaaggctttaactgaattggttgaagatgctagagctaagggtgctgagttgtggcaacccgatactgctttgccaaacaaaggtttctttttccctcctacattagttttgaaaccaacattggacatgagggtcatgcaagaagaaccatttggtccaattgcaattatggacgagttctctgagttggcagacgctgtcaaggaggctaatcgtttgccttacggtttagctgcttatgtttatactagatgtgagaggactgaaagattgttgggtgagaagttggaggctggtatggttgctattaaccaccacggtattggtacaccagaggtcccattcggtggtattaaggactctggttacggtacagaaggaggaccagctgcattgcgtgctcatactattattaagttggtttcttctttgcatgagagaccaaacttttaacgatcgtaagcggcgaatctctggctcatgggggatatcctctttgtttggcttttttttcccattctctgttttgattatctaatgactcattgggaggattttctcacttcaagcttttttttcttgcactctttcataactccagctctctctaactgaggctacaatgccttttaacgaacttatgagacgtttctaaattatataggtatatgccaatatataattacacataaaaataaatataaataaaatataaaaataaaaataaacatcgaaaaagaagatgtgaaattgcgaagactagaaagcacaaacgagcggtctatatcggcgactcgaggctctacaagcctcatatgggttcaatgggtctgcaatgaccgcatacggacttggacaattaccttctattgaatttctgagaagagatacatctgaccagcaatgtaagcagacaatcccaattctgtaaacaacctctttgtccataattccccatcagaagagtgaaaaatgccctcaaaacgcatgcgccactcccacctctcagctgcactgcgccacctctgagggtcctttcaggggtcgactaccccggacacctcgcagaggagcgagatcacgtacttttaaaatggcagagacgcgcagtttcttgaagaaaggataaaaatgaaatggtgcggaaatgcgaaaatgatgaaaaattttcttggtggcgaggaaattgagtgcaataattggcacgaggttgttgccacccgagtgtgagtatatatcctagtttctgcacttttcttcttcttttccttgcgttttcttttcaactttttttactttttccttcaacagacaaatctaacttatatatcacatctagaatgcacatgtctgagactcctattaacactcaattattgattgctggtcaatggcaagacgctgctgacggtagaactttgccaattttagatccatctaatggtgaattgattggtaaagtcgcacacgcttctgtcgcagatttggataagacagttgctggtgcagctgctggttacgacgcttggagaaacttgactgcatgggatcgttacactattatgaggagggctgctgagttgttaagggagagagctgatgctattggtagaattatgtcaagagagcaaggtaagccattggctcaagctgttattgaaattaatgctgctgctggtgtcattgagtacttcggagaggaaggtagaaggttgaacgatgagttggtcccagctcgtgctccttttgtcgagcagagagtcttgcatcgtccaataggtgtcgttgcagctttcactccatggaactggccaattaatcaaattgctagaaagttgggagctgctttaggtgctggttgtggtgtcatagtcaaggctccagaagacacacccgcttctcccgctgagttgattagatgtttctgtgatgctggtattcccgctggtgcagtctctttggtttacggtactcccgctgaaatttcagaatatttgattgctcatccagttgttagaaaaatttctttcactggttctacacccgttggtaagcacttggctgcattggctggtgcacaaatgaagccagtcacaatggaattgggtggtcacggtcccgttattgtctgcagagatgctgacttagatcaagctgctgaaagggttgcagctgctaagtttcgtaacgctggtcaagtttgcactgcaccaactagatttttattgcataaagatattgcttctgcttttattgaaaagtttaaagctcaaatgtcagctttaaagataggttgcggtttggatgagggtgtcacaatgggtccattaataaacgaaagaagagttaaggctttaactgaattggttgaagatgctagagctaagggtgctgagttgtggcaacccgatactgctttgccaaacaaaggtttctttttccctcctacattagttttgaaaccaacattggacatgagggtcatgcaagaagaaccatttggtccaattgcaattatggacgagttctctgagttggcagacgctgtcaaggaggctaatcgtttgccttacggtttagctgcttatgtttatactagatgtgagaggactgaaagattgttgggtgagaagttggaggctggtatggttgctattaaccaccacggtattggtacaccagaggtcccattcggtggtattaaggactctggttacggtacagaaggaggaccagctgcattgcgtgctcatactattattaagttggtttcttctttgcatgagagaccaaacttttaattaattaagtatagccatatagtttaattcctttatactttttataactatttcttacactaattattattatcaattatttattgtagaacttgactcttgcgtcgatcaccatgacagggctatcttaacaaggggtaatttttgttgatggagtcaagtagcattccgacgggaagtgtcgatgcctctgaacgaaatcttccgattagctctgcaaagaagtggaaattgtcagcgcagaattcgccgaaaaggtctatgagggaaccgagccattgtatggtaccgatattgcagaattgattctatttgcagtttctacacctcaaaacactgttattgcagaaacacttgtttttgctagtaaccaagcttctgcttaccatattttcagaggatcattagataaatagatttgatataaacgcttctataataataaataacatcaacaagtgactaaccccaagtatccaattttagacctaatgcctgaaagtgccttgcacgatttaggattcggcaccagaaagtttgtgccgtgctcgcttattggacttgtatagtgataaggcaaaaaaaaaaaacttgaaagtacttgcgtaactcagaggttgccttttcgggcc

SEQ ID NO: 45 - protein, 486 aa, Artificial Sequence
  A synthetic amino acid sequence
MHMSETPINTQLLIAGQWQDAADGRTLPILDPSNGELIGKVAHASVADLDKTVAGAAAGYDAWRNLTAWDRYTIMRRAAELLRERADAIGRIMSREQGKPLAQAVIEINAAAGVIEYFGEEGRRLNDELVPARAPFVEQRVLHRPIGVVAAFTPWNWPINQIARKLGAALGAGCGVIVKAPEDTPASPAELIRCFCDAGIPAGAVSLVYGTPAEISEYLIAHPVVRKISFTGSTPVGKHLAALAGAQMKPVTMELGGHGPVIVCRDADLDQAAERVAAAKFRNAGQVCTAPTRFLLHKDIASAFIEKFKAQMSALKIGCGLDEGVTMGPLINERRVKALTELVEDARAKGAELWQPDTALPNKGFFFPPTLVLKPTLDMRVMQEEPFGPIAIMDEFSELADAVKEANRLPYGLAAYVYTRCERTERLLGEKLEAGMVAINHHGIGTPEVPFGGIKDSGYGTEGGPAALRAHTIIKLVSSLHERPNF

SEQ ID NO: 46 - DNA, 5676 bp, Artificial Sequence
  A synthetic oligonucleotide sequence
ccacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactggatcattatgactccaggggttggtttagatgacaaaggtgatgcacttggtcaacaatatagaactgttgatgaagttgtaaagactggaacggatatcataattgttggtagaggtttgtacggtcaaggaagagatcctatagagcaagctaaaagataccaacaagctggttggaatgcttatttaaacagatttaaatgattcttacacaaagatttgatacatgtacactagtttaaataagcatgaaaagaattacacaagcaaaaaaaaaaaaataaatgaggtactttacgttcacctacaaccaaaaaaactagatagagtaaaatcttaagatttagaaaaagttgtttaacaaaggctttagtatgtgaatttttaatgtagcaaagcgataactaataaacataaacaaaagtatggttttctttatcagtcaaatcattatcgattgattgttccgcgtatctgcagataacttcgtataatgtatgctatacgaagttatagatcgcggccgctaacctgatccaaaaggggtatgtctattttttagagtgtgtctttgtgtcaaattatggtagaatgtgtaaagtagtataaactttcctctcaaatgacgaggtttaaaacaccccccgggtgagccgagccgagaatggggcaattgttcaatgtgaaatagaagtatcgagtgagaaacttgggtgttggccagccaagggggaaggaaaatggcgcgaatgctcaggtgagattgttttggaattgggtgaagcgaggaaatgagcgacccggaggttgtgactttagtggcggaggaggacggaggaaaagccaagagggaagtgtatataaggggagcaatttgccaccaggatagaattggatgagttataattctactgtatttattgtataatttatttctccttttatatcaaacacattacaaaacacacaaaacacacaaacaaacacaattacaaaaagctagcatgcacatgtctgagactcctattaacactcaattattgattgctggtcaatggcaagacgctgctgacggtagaactttgccaattttagatccatctaatggtgaattgattggtaaagtcgcacacgcttctgtcgcagatttggataagacagttgctggtgcagctgctggttacgacgcttggagaaacttgactgcatgggatcgttacactattatgaggagggctgctgagttgttaagggagagagctgatgctattggtagaattatgtcaagagagcaaggtaagccattggctcaagctgttattgaaattaatgctgctgctggtgtcattgagtacttcggagaggaaggtagaaggttgaacgatgagttggtcccagctcgtgctccttttgtcgagcagagagtcttgcatcgtccaataggtgtcgttgcagctttcactccatggaactggccaattaatcaaattgctagaaagttgggagctgctttaggtgctggttgtggtgtcatagtcaaggctccagaagacacacccgcttctcccgctgagttgattagatgtttctgtgatgctggtattccagctggtgcagtctctttggtttacggtactcccgctgaaatttcagaatatttgattgctcatccagttgttagaaaaatttctttcactggttctacacccgttggtaagcacttggctgcattggctggtgcacaaatgaagccagtcacaatggaattgggtggtcacggtcccgttattgtctgcagagatgctgacttagatcaagctgctgaaagggttgcagctgctaagtttcgtaacgctggtcaagtttgcattgcaccaactagatttttattgcataaagatattgcttctgcttttattgaaaagtttaaagctcaaatgtcagctttaaagataggttgcggtttggatgagggtgtcacaatgggtccattaataaacgaaagaagagttaaggctttaactgaattggttgaagatgctagagctaagggtgctgagttgtggcaacccgatactgctttgccaaacaaaggtttctttttccctcctacattagttttgaaaccaacattggacatgagggtcatgcaagaagaaccatttggtccaattgcaattatggacgagttctctgagttggcagacgctgtcaaggaggctaatcgtttgccttacggtttagctgcttatgtttatactagatgtgagaggactgaaagattgttgggtgagaagttggaggctggtatggttgctattaaccaccacggtattggtacaccagaggtcccattcggtggtattaaggactctggttacggtacagaaggaggaccagctgcattgcgtgctcatactattattaagttggtttcttctttgcatgagagaccaaacttttaacgatcgtaagcggcgaatctctggctcatgggggatatcctctttgtttggcttttttttcccattctctgttttgattatctaatgactcattgggaggattttctcacttcaagcttttttttcttgcactctttcataactccagctctctctaactgaggctacaatgccttttaacgaacttatgagacgtttctaaattatataggtatatgccaatatataattacacataaaaataaatataaataaaatataaaaataaaaataaacatcgaaaaagaagatgtgaaattgcgaagactagaaagcacaaacgagcggtctatatcggcgactcgaggctctacaagcctcatatgggttcaatgggtctgcaatgaccgcatacggacttggacaattaccttctattgaatttctgagaagagatacatctgaccagcaatgtaagcagacaatcccaattctgtaaacaacctctttgtccataattccccatcagaagagtgaaaaatgccctcaaaacgcatgcgccactcccacctctcagctgcactgcgccacctctgagggtcctttcaggggtcgactaccccggacacctcgcagaggagcgagatcacgtacttttaaaatggcagagacgcgcagtttcttgaagaaaggataaaaatgaaatggtgcggaaatgcgaaaatgatgaaaaattttcttggtggcgaggaaattgagtgcaataattggcacgaggttgttgccacccgagtgtgagtatatatcctagtttctgcacttttcttcttcttttccttgcgttttcttttcaactttttttactttttccttcaacagacaaatctaacttatatatcacatctagaatgcacatgtctgagactcctattaacactcaattattgattgctggtcaatggcaagacgctgctgacggtagaactttgccaattttagatccatctaatggtgaattgattggtaaagtcgcacacgcttctgtcgcagatttggataagacagttgctggtgcagctgctggttacgacgcttggagaaacttgactgcatgggatcgttacactattatgaggagggctgctgagttgttaagggagagagctgatgctattggtagaattatgtcaagagagcaaggtaagccattggctcaagctgttattgaaattaatgctgctgctggtgtcattgagtacttcggagaggaaggtagaaggttgaacgatgagttggtcccagctcgtgctccttttgtcgagcagagagtcttgcatcgtccaataggtgtcgttgcagctttcactccatggaactggccaattaatcaaattgctagaaagttgggagctgctttaggtgctggttgtggtgtcatagtcaaggctccagaagacacacccgcttctcccgctgagttgattagatgtttctgtgatgctggtattcccgctggtgcagtctctttggtttacggtactcccgctgaaatttcagaatatttgattgctcatccagttgttagaaaaatttctttcactggttctacacccgttggtaagcacttggctgcattggctggtgcacaaatgaagccagtcacaatggaattgggtggtcacggtcccgttattgtctgcagagatgctgacttagatcaagctgctgaaagggttgcagctgctaagtttcgtaacgctggtcaagtttgcattgcaccaactagatttttattgcataaagatattgcttctgcttttattgaaaagtttaaagctcaaatgtcagctttaaagataggttgcggtttggatgagggtgtcacaatgggtccattaataaacgaaagaagagttaaggctttaactgaattggttgaagatgctagagctaagggtgctgagttgtggcaacccgatactgctttgccaaacaaaggtttctttttccctcctacattagttttgaaaccaacattggacatgagggtcatgcaagaagaaccatttggtccaattgcaattatggacgagttctctgagttggcagacgctgtcaaggaggctaatcgtttgccttacggtttagctgcttatgtttatactagatgtgagaggactgaaagattgttgggtgagaagttggaggctggtatggttgctattaaccaccacggtattggtacaccagaggtcccattcggtggtattaaggactctggttacggtacagaaggaggaccagctgcattgcgtgctcatactattattaagttggtttcttctttgcatgagagaccaaacttttaattaattaagtatagccatatagtttaattcctttatactttttataactatttcttacactaattattattatcaattatttattgtagaacttgactcttgcgtcgatcaccatgacagggctatcttaacaaggggtaatttttgttgatggagtcaagtagcattccgacgggaagtgtcgatgcctctgaacgaaatcttccgattagctctgcaaagaagtggaaattgtcagcgcagaattcgccgaaaaggtctatgagggaaccgagccattgtatggtaccgatattgcagaattgattctatttgcagtttctacacctcaaaacactgttattgcagaaacacttgtttttgctagtaaccaagcttctgcttaccatattttcagaggatcattagataaatagatttgatataaacgcttctataataataaataacatcaacaagtgactaaccccaagtatccaattttagacctaatgcctgaaagtgccttgcacgatttaggattcggcaccagaaagtttgtgccgtgctcgcttattggacttgtatagtgataaggcaaaaaaaaaaaacttgaaagtacttgcgtaactcagaggttgccttttcgggcc

SEQ ID NO: 47 - DNA, 5700 bp, Artificial Sequence
  A synthetic oligonucleotide sequence
ccacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactggatcattatgactccaggggttggtttagatgacaaaggtgatgcacttggtcaacaatatagaactgttgatgaagttgtaaagactggaacggatatcataattgttggtagaggtttgtacggtcaaggaagagatcctatagagcaagctaaaagataccaacaagctggttggaatgcttatttaaacagatttaaatgattcttacacaaagatttgatacatgtacactagtttaaataagcatgaaaagaattacacaagcaaaaaaaaaaaaataaatgaggtactttacgttcacctacaaccaaaaaaactagatagagtaaaatcttaagatttagaaaaagttgtttaacaaaggctttagtatgtgaatttttaatgtagcaaagcgataactaataaacataaacaaaagtatggttttctttatcagtcaaatcattatcgattgattgttccgcgtatctgcagataacttcgtataatgtatgctatacgaagttatagatcgcggccgctaacctgatccaaaaggggtatgtctattttttagagtgtgtctttgtgtcaaattatggtagaatgtgtaaagtagtataaactttcctctcaaatgacgaggtttaaaacaccccccgggtgagccgagccgagaatggggcaattgttcaatgtgaaatagaagtatcgagtgagaaacttgggtgttggccagccaagggggaaggaaaatggcgcgaatgctcaggtgagattgttttggaattgggtgaagcgaggaaatgagcgacccggaggttgtgactttagtggcggaggaggacggaggaaaagccaagagggaagtgtatataaggggagcaatttgccaccaggatagaattggatgagttataattctactgtatttattgtataatttatttctccttttatatcaaacacattacaaaacacacaaaacacacaaacaaacacaattacaaaaagctagcatgactagaacttttgaattgggtgaattgattcgttctgataacttcattgatggtgcttggactccagcacaagacaacttgaggttcgctgtcactaacccagcttctggagagataattgctgaggtcgctgactcttctccagctgatgcaagagctgctactgatgcagctgcaagagctttgccagcttggagggctagattgccaaaagagagagctgcagtcttgcatcgttggcacgctttgataatggctaacttggatgcattgggtgctttaatatctttggagcaaggtaaacctttggctgagggtaagggtgaggtcgcttatggtgcttcttacgtcgcatggttcgcagaggaagcaacaagaatttacggtgatttgattcctcagcaacagaggggtaagaggatgactgctgtcaaagagccagtcggagtcgttgctgctattacaccatggaattttccattggcaatgattgcaagaaagatagcacccgctttggcagctggttgtactgttgtcgctaagccagctgaggacactccattgactgcttcagctttggtcttgttggctcacgaagctggtgttccacccggtgttttgaacttgattactgcatcccgtgatcatgctgtcgcagcagtcgctgagtggttgcatgacgctagagttagaaaaattacttttactggatcaactccagtcggtaagtacttggctagggaatctgctgaaactttaaagaagttatctttggagttgggtggtaatgctccatttattgtttttgacgatgctgacttggaggctgcagtcgctggtttgatggctgctaagtttcgtaacggtggtcagacttgtgtctgtccaaatcgtgtctacgtccaagctggtgtctacgagaggtttggtgctttgttggctgaaagggttggtgctttgaaggttgctccagcaactgatccagctgctcaaattggtccaatgattaattctagggcattggacaagattgctaggcacgtcgatgacgctgttgcacatggtgctagagtcttgactggtggtaagaggttggcagagttgggtccacactactacgctccaactgttttggctgacgcaacagcagcaatgcagttgaactcagaggaaactttcggtccaatagtcccattgtttcgtttcgaggacgaggctgaagcagttaacgctgctaacgacactccattcggtttagcagcttatttttattctgaaggtgtcaaaagaattgatagggtcgctagggctttggaagctggtattgttggtataaatgaaggtgcagtcgcttcagaagctgctccattcggtggtgttaaggaatctggttacggtagagaaggttctaaatatggtttggatgattatttgtctattaaatatttatgtcaaggtaatttagaataacgatcgtaagcggcgaatctctggctcatgggggatatcctctttgtttggcttttttttcccattctctgttttgattatctaatgactcattgggaggattttctcacttcaagcttttttttcttgcactctttcataactccagctctctctaactgaggctacaatgccttttaacgaacttatgagacgtttctaaattatataggtatatgccaatatataattacacataaaaataaatataaataaaatataaaaataaaaataaacatcgaaaaagaagatgtgaaattgcgaagactagaaagcacaaacgagcggtctatatcggcgactcgaggctctacaagcctcatatgggttcaatgggtctgcaatgaccgcatacggacttggacaattaccttctattgaatttctgagaagagatacatctgaccagcaatgtaagcagacaatcccaattctgtaaacaacctctttgtccataattccccatcagaagagtgaaaaatgccctcaaaacgcatgcgccactcccacctctcagctgcactgcgccacctctgagggtcctttcaggggtcgactaccccggacacctcgcagaggagcgagatcacgtacttttaaaatggcagagacgcgcagtttcttgaagaaaggataaaaatgaaatggtgcggaaatgcgaaaatgatgaaaaattttcttggtggcgaggaaattgagtgcaataattggcacgaggttgttgccacccgagtgtgagtatatatcctagtttctgcacttttcttcttcttttccttgcgttttcttttcaactttttttactttttccttcaacagacaaatctaacttatatatcacatctagaatgactagaacttttgaattgggtgaattgattcgttctgataacttcattgatggtgcttggactccagcacaagacaacttgaggttcgctgtcactaacccagcttctggagagataattgctgaggtcgctgactcttctccagctgatgcaagagctgctactgatgcagctgcaagagctttgccagcttggagggctagattgccaaaagagagagctgcagtcttgcatcgttggcacgctttgataatggctaacttggatgcattgggtgctttaatatctttggagcaaggtaaacctttggctgagggtaagggtgaggtcgcttatggtgcttcttacgtcgcatggttcgcagaggaagcaacaagaatttacggtgatttgattcctcagcaacagaggggtaagaggatgactgctgtcaaagagccagtcggagtcgttgctgctattacaccatggaattttccattggcaatgattgcaagaaagatagcacccgctttggcagctggttgtactgttgtcgctaagccagctgaggacactccattgactgcttcagctttggtcttgttggctcacgaagctggtgttccacccggtgttttgaacttgattactgcatcccgtgatcatgctgtcgcagcagtcgctgagtggttgcatgacgctagagttagaaaaattacttttactggatcaactccagtcggtaagtacttggctagggaatctgctgaaactttaaagaagttatctttggagttgggtggtaatgctccatttattgtttttgacgatgctgacttggaggctgcagtcgctggtttgatggctgctaagtttcgtaacggtggtcagacttgtgtctgtccaaatcgtgtctacgtccaagctggtgtctacgagaggtttggtgctttgttggctgaaagggttggtgctttgaaggttgctccagcaactgatccagctgctcaaattggtccaatgattaattctagggcattggacaagattgctaggcacgtcgatgacgctgttgcacatggtgctagagtcttgactggtggtaagaggttggcagagttgggtccacactactacgctccaactgttttggctgacgcaacagcagcaatgcagttgaactcagaggaaactttcggtccaatagtcccattgtttcgtttcgaggacgaggctgaagcagttaacgctgctaacgacactccattcggtttagcagcttatttttattctgaaggtgtcaaaagaattgatagggtcgctagggctttggaagctggtattgttggtataaatgaaggtgcagtcgcttcagaagctgctccattcggtggtgttaaggaatctggttacggtagagaaggttctaaatatggtttggatgattatttgtctattaaatatttatgtcaaggtaatttagaataattaattaagtatagccatatagtttaattcctttatactttttataactatttcttacactaattattattatcaattatttattgtagaacttgactcttgcgtcgatcaccatgacagggctatcttaacaaggggtaatttttgttgatggagtcaagtagcattccgacgggaagtgtcgatgcctctgaacgaaatcttccgattagctctgcaaagaagtggaaattgtcagcgcagaattcgccgaaaaggtctatgagggaaccgagccattgtatggtaccgatattgcagaattgattctatttgcagtttctacacctcaaaacactgttattgcagaaacacttgtttttgctagtaaccaagcttctgcttaccatattttcagaggatcattagataaatagatttgatataaacgcttctataataataaataacatcaacaagtgactaaccccaagtatccaattttagacctaatgcctgaaagtgccttgcacgatttaggattcggcaccagaaagtttgtgccgtgctcgcttattggacttgtatagtgataaggcaaaaaaaaaaaacttgaaagtacttgcgtaactcagaggttgccttttcgggcc

SEQ ID NO: 48 - protein, 1180 aa, Issachenkia orientalis
MSTVEDHSSLHKLRKESEILSNANKILVANRGEIPIRIFRSAHELSMHTVAIYSHEDRLSMHRLKADEAYAIGKTGQYSPVQAYLQIDEIIKIAKEHDVSMIHPGYGFLSENSEFAKKVEESGMIWVGPPAEVIDSVGDKVSARNLAIKCDVPVVPGTDGPIEDIEQAKQFVEQYGYPVIIKAAFGGGGRGMRVVREGDDIVDAFQRASSEAKSAFGNGTCFIERFLDKPKHIEVQLLADNYGNTIHLFERDCSVQRRHQKVVEIAPAKTLPVEVRNAILKDAVTLAKTANYRNAGTAEFLVDSQNRHYFIEINPRIQVEHTITEEITGVDIVAAQIQIAAGASLEQLGLLQNKITTRGFAIQCRITTEDPAKNFAPDTGKIEVYRSAGGNGVRLDGGNGFAGAVISPHYDSMLVKCSTSGSNYEIARRKMIRALVEFRIRGVKTNIPFLLALLTHPVFISGDCWTTFIDDTPSLFEMVSSKNRAQKLLAYIGDLCVNGSSIKGQIGFPKLNKEAEIPDLLDPNDEVIDVSKPSTNGLRPYLLKYGPDAFSKKVREFDGCMIMDTTWRDAHQSLLATRVRTIDLLRIAPTTSHALQNAFALECWGGATFDVAMRFLYEDPWERLRQLRKAVPNIPFQMLLRGANGVAYSSLPDNAIDHFVKQAKDNGVDIFRVFDALNDLEQLKVGVDAVKKAGGVVEATVCYSGDMLIPGKKYNLDYYLETVGKIVEMGTHILGIKDMAGTLKPKAAKLLIGSIRSKYPDLVIHVHTHDSAGTGISTYVACALAGADIVDCAINSMSGLTSQPSMSAFIAALDGDIETGVPEHFARQLDAYWAEMRLLYSCFEADLKGPDPEVYKHEIPGGQLTNLIFQAQQVGLGEQWEETKKKYEDANMLLGDIVKVTPTSKVVGDLAQFMVSNKLEKEDVEKLANELDFPDSVLDFFEGLMGTPYGGFPEPLRTNVISGKRRKLKGRPGLELEPFNLEEIRENLVSRFGPGITECDVASYNMYPKVYEQYRKVVEKYGDLSVLPTKAFLAPPTIGEEVHVEIEQGKTLIIKLLAISDLSKSHGTREVYFELNGEMRKVTIEDKTAAIETVTRAKADGHNPNEVGAPMAGVVVEVRVKHGTEVKKGDPLAVLSAMKMEMVISAPVSGRVGEVFVNEGDSVDMGDLLVKIAKDEAPAA

SEQ ID NO: 49 - protein, 419 aa, Issachenkia orientalis
MSRGFFTENITQLPPDPLFGLKARFSNDSRENKVDLGIGAYRDDNGKPWILPSVRLAENLIQNSPDYNHEYLPIGGLADFTSAAARVVFGGDSKAISQNRLVSIQSLSGTGALHVAGLFIKRQYKSLDGTSEDPLIYLSEPTWANHVQIFEVIGLKPVFYPYWHAASKTLDLKGYLKAINDAPEGSVFVLHATAHNPTGLDPTQEQWMEILAAISAKKHLPLFDCAYQGFTSGSLDRDAWAVREAVNNDKYEFPGIIVCQSFAKNVGMYGERIGAVHIVLPESDASLNSAIFSQLQKTIRSEISNPPGYGAKIVSKVLNTPELYKQWEQDLITMSSRITAMRKELVNELERLGTPGTWRHITEQQGMFSFTGLNPEQVAKLEKEHGVYLVRSGRASIAGLNMGNVKYVAKAIDSVVRDL

SEQ ID NO: 50 - protein, 344 aa, Issachenkia orientalis
MSKPKVLLIGFGGVGTIVSYTLEHLGRAEVSAVSRPETHDSIVNGFRIESIDYGIVENYVPTNVYVTAKEAYKQQGPFDYIIITTKNIPDIAPVVDMIDGCYNEKSVIVLIQNGIGIEIPIYRRYPNAIILSGVTLIGTTLYEATVKHVARDDIKFGPFINYNLDKQLQINKCKEFIELYENDKNLVEYEEDVKFTRWRKLVYNACINTTCALANLDAGRVQIFGGFETLVKPAMLEVIAVAKSEGVELPAKEVMDTMCNMGKDVYYPPSMLIDVRNGTYLEHIVIIGNVVKYGSRNGVPIPTLTVLNNLLKLVQMRTMEANKRFVLPEKRPLPEENYQIEYLY

SEQ ID NO: 51 - DNA, 1438 bp, Danaus plexippus
atgagagttgattccaagatcattgtcaagaaagagactcaagacgaaaacttgtatcaatccttgcaaggttgttgagtgggctgcaccagacgagattaagaaagcaattgacttaaagccacgtttgggtcctgcttctcacgatgagttgttggcctttatggcaaacgttgctaggtattctgtcaatactggtcatccatacttcgttaaccagttattctcatccgttgatccatatggtttagttggtcaatggttaaccgacgcattgaacccatccgtttacacctttgaagtcgctcctgtctttaccttgatggaggaagaggttttgcgtgaaatgcgtaagattgtcggttggcctgagggcgaaggtgatggtatcttctgcccaggtggttctatcgccaacggttacgctatttcctgtgctagacatcacttttacccagaggtcaagtataagggtgttcatgctgttcctaagttagttttgtttacctccgaactcgcgcactacagtaccaaaaagatggctgctttcatgggtattggttccgataactgtgttaacattaagaccgatgatgttggtaagatgaacatcgttgatttagaaatgaagatcaagattgctattgacaacaagtgtaccccattcatggttacagctacctctggtactaccgttttcggtgctttcgacccattagtcgcaatctccgatctttgcaagaagtacaatctgtggttgcatgtcgatgctgcttggggtggtggtgctctcatgtctaagaagcacagacaccttttgaacggtatcgaattagccgactctgttacttggaaccctcataagcttttagccgctccacaacaatgttccacctttttgaccagacacaagaaagttctatccgaaggtcattcatccaatgcaaagtacttattccaaaaagacaagttttacgacacatcctacgacactggtgataaacatattcaatgtggtagaagagcagatgttcttaagttctggttcatgtggaaggctaagggtactgaaggtttcgagaaacacgttgacaagctttttgataacgcaaagtacttcttagaccacattaagcagagagaaggtttccaattggttattgcagaaccacaatgcaccaatatcatgttctggtacatcccaaagtgcctgagaggttgtgaaaatgatgcagactactatgagagattacataaagttgcgcctaagatcaaggaaagaatgatcaaggaaggttcaatgatggtcacctaccaaccacaaggtgatcttgttaacttctttagaattgtttttcagaactctgcattggaccataaggacatggtttacttcgcaaatgaattcgaaagattgggctccgatatgatcgtttaa

(19) United States
(12) Patent Application Publication
(10) Pub. No.: US 2023/0203430 A1
(43) Pub. Date: Jun. 29, 2023

(54) FERMENTATION PATHWAY FOR PRODUCING MALONIC ACID

(71) Applicant: CARGILL, INCORPORATED, Wayzata, MN (US)

(72) Inventors: Keith Michael BRADY, Eden Prairie, MN (US); Cathy Staloch HASS, Plymouth, MN (US); Peter Alan JAUERT, Minneapolis, MN (US); Nandita KOHLI, Plymouth, MN (US); Briana Kristen KOZLOWICZ, Hopkins, MN (US); Hans H. LIAO, Plymouth, MN (US); Michael Tai Man LOUIE, Camas, WA (US); Michiel MATTHIJS, Gent (BE); Naga Sirisha PARIMI, Minnetonka, MN (US); Catherine Bradshaw POOR, Minneapolis, MN (US); Brian Jeffrey RUSH, Minneapolis, MN (US); Travis Tobert WOLTER, Minneapolis, MN (US)

(73) Assignee: CARGILL, INCORPORATED, Wayzata, MN (US)

(21) Appl. No.: 17/757,891

(22) PCT Filed: Dec. 21, 2020
(86) PCT No.: PCT/US2020/066372

§ 371 Date: Jun. 23, 2022

Related U.S. Application Data
(60) Provisional application No. 62/952,967, filed on Dec. 23, 2019.

(51) Int. Cl. C12N 1/16; C12N 15/52; C12N 15/81; C12N 9/02

(52) U.S. Cl. CPC C12N 1/16; C12N 9/0008; C12N 15/52; C12N 15/81; C12Y 102/01027; C12R 2001/84

(57) ABSTRACT

The present disclosure provides an engineered microorganism capable of producing malonic acid, malonate, esters of malonic acid, or mixtures thereof. The engineered microorganism includes a malonate-semialdehyde dehydrogenase that is heterologous to a native form of the engineered microorganism and comprises at least 90% sequence identity to any one of SEQ ID Nos: 7, 9, 11, 13, 15, 17, 19, 21, 23, 27, 29, and 31, wherein the engineered microorganism is capable of producing about 9 g/L to about 250 g/L of malonic acid, malonate, esters of malonic acid, or mixtures thereof.

### CROSS-REFERENCE TO RELATED APPLICATION

This application claims the benefit of priority to U.S. Provisional Pat. Application Serial No. 62/952,967 entitled “FERMENTATION PATHWAY FOR PRODUCING MALONIC ACID,” filed Dec. 23, 2019, the disclosure of which is incorporated herein in its entirety by reference.

### INCORPORATION BY REFERENCE OF SEQUENCE LISTING

A Sequence Listing is provided herewith as a text file, “4361.133WO1 SEQ LIST in CRF/TXT/ST25” and submitted as “2101331.txt” created on Dec. 18, 2020 and having a size of 321,915 bytes. The contents of the text file are incorporated by reference herein in their entirety.

### BACKGROUND

Fermentation processes are used commercially at large scale to produce organic molecules such as ethanol, citric acid and lactic acid. In those processes, a carbohydrate is fed to an organism that is capable of metabolizing it to the desired fermentation product. The carbohydrate and organism are selected together so that the organism is capable of efficiently digesting the carbohydrate to form the product that is desired in good yield. It is becoming more common to use genetically engineered organisms in these processes, in order to optimize yields and process variables, or to enable particular carbohydrates to be metabolized.

### SUMMARY OF THE DISCLOSURE

The present disclosure provides an engineered microorganism capable of producing malonic acid, malonate, esters of malonic acid, or mixtures thereof. The engineered microorganism includes a malonate-semialdehyde dehydrogenase (MSADh) that is heterologous to a native form of the engineered microorganism and comprises at least 90% sequence identity to any one of SEQ ID Nos: 7, 9, 11, 13, 15, 17, 19, 21, 23, 27, 29, and 31, wherein the engineered microorganism is capable of producing about 9 g/L to about 250 g/L of malonic acid, malonate, esters of malonic acid, or mixtures thereof.

The present disclosure further provides an engineered microorganism capable of producing malonic acid, malonate, esters of malonic acid, or mixtures thereof. The engineered microorganism includes a malonate-semialdehyde dehydrogenase that is heterologous to the native form of the engineered microorganism and comprises at least 90% sequence identity to SEQ ID No: 11. The amino acid residue of the polypeptide that aligns with amino acid residue 160 of SEQ ID NO: 11 is not phenylalanine and the engineered microorganism is capable of producing about 9 g/L to about 250 g/L of malonic acid, malonate, esters of malonic acid, or mixtures thereof.

The present disclosure further provides an engineered microorganism capable of producing malonic acid, malonate, esters of malonic acid, or mixtures thereof. The engineered microorganism includes a malonate-semialdehyde dehydrogenase that is heterologous to the native form of the engineered microorganism and comprises at least 90% sequence identity to SEQ ID No: 11. The amino acid residue of the polypeptide that aligns with amino acid residue 160 of SEQ ID NO: 11 is tryptophan and the engineered microorganism is capable of producing about 9 g/L to about 250 g/L of malonic acid, malonate, esters of malonic acid, or mixtures thereof.

The present disclosure further provides a fermentation method for producing malonic acid, malonate, esters of malonic acid, or mixtures thereof. The method includes culturing an engineered microorganism including a heterologous malonate-semialdehyde dehydrogenase. The engineered microorganism includes at least 90% sequence identity to any one of SEQ ID Nos: 7, 9, 11, 13, 15, 17, 19, 21, 23, 27, 29, and 31 in the presence of a medium comprising at least one carbon source. The method further includes producing about 9 g/L to about 250 g/L of malonic acid, malonate, esters of malonic acid, or mixtures thereof.

The present disclosure further provides a malonate-semialdehyde dehydrogenase formed according to a method that includes culturing an engineered microorganism in the presence of a medium comprising at least one carbon source. The method further includes isolating malonic acid, malonate, esters of malonic acid, or mixtures thereof from the culture. The engineered microorganism includes a malonate-semialdehyde dehydrogenase that is heterologous to a native form of the engineered microorganism and comprises at least 90% sequence identity to any one of SEQ ID Nos: 7, 9, 11, 13, 15, 17, 19, 21, 23, 27, 29, and 31, wherein the engineered microorganism is capable of producing about 9 g/L to about 250 g/L of malonic acid, malonate, esters of malonic acid, or mixtures thereof.

### BRIEF DESCRIPTION OF THE FIGURES

The drawings illustrate generally, by way of example, but not by way of limitation, various embodiments discussed in the present document.

FIG. 1 is a flow diagram showing a metabolic pathway for forming malonic acid, malonate, esters of malonic acid, or mixtures thereof, in accordance with various embodiments.

### DETAILED DESCRIPTION

Reference will now be made in detail to various examples of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order without departing from the principles of the disclosure, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of or mostly, as in at least about 50%, 60%, 70%, 80%, 90%, 95%, 96%, 97%, 98%, 99%, 99.5°%, 99.9%, 99.99%, or at least about 99.999% or more, or 100%.

Various abbreviations are used herein. Abbreviations and their meaning can include 3-HP, 3-hydroxypropionic acid, 3-HPA, 3-hydroxypropionaldehyde; 3-HPDH, 3-hydroxypropionic acid dehydrogenase; AAM, alanine 2,3 aminomutase; AAT, aspartate aminotransferase; ACC, acetyl-CoA carboxylase; ADC, aspartate 1-decarboxylase; AKG, alpha-ketoglutarate; ALD, aldehyde dehydrogenase; BAAT, β-alanine aminotransferase; BCKA, branched-chain alpha-keto acid decarboxylase; CYB2, L-(+)-lactate-cytochronie c oxidoreductase; CYC, iso-2-cytochrome c; EMS, ethane methyl sulfonase; ENO, enolase; gabT, 4-aminobutyrate aminotransferase; GAPDH, glyceraldehyde-3-phosphate dehydrogenase 3; GPD, glycerol 3-phosphate dehydrogenase; GPP, glycerol 3-phosphate phosphatase; HIBADH, 3-hydroxyisobutyrate dehydrogenase; IPDA, indolepyruvate decarboxylase; KGD, alpha-ketoglutarate decarboxylase; LDH, lactate dehydrogenase; MAE, malic enzyme; OAA, oxaloacetate; PCK, phosphoenolpyruvate carboxykinase; PDC, pyruvate decarboxylase; PDH, pyruvate dehydrogenase; PEP, phosphoenolpyruvate; PGK, phosphoglycerate kinase; PPC, phosphoenolpyruvate carboxylase; PYC, pyruvate carboxylase; RKI, ribose 5-phosphate ketol-isomerase; TAL, transaldolase; TEF1, translation elongation factor-1; TEF2, translation elongation factor-2; TKL, transketolase, XDH, xylitol dehydrogenase; XR, xylose reductase, YP, yeast extract/peptone.

Various embodiments of the present disclosure relate to an engineered microorganism capable of producing malonic acid, malonate and esters of malonic acid. As understood herein, a malonate includes a mono-anion and di-anion of malonic acid, esters of malonic acid can include mono-esters and di-esters. As further understood herein, in some examples the engineered microorganism may produce malonic acid or malonate that is capable of being modified to produce the corresponding ester form of the malonic acid or malonate. Alternativity, the ester can be produced by a separate procedure outside of the pathway. As described further herein, the engineered microorganism can include a malonate-semialdehyde dehydrogenase that is heterologous to a native form of the engineered microorganism and comprises at least 90% sequence identity to any one of SEQ ID Nos: 7, 9, 11, 13, 15, 17, 19, 21, 23, 27, 29, and 31. According to various embodiments, the engineered microorganisms described herein are capable of producing about 9 g/L to about 250 g/L of malonic acid, malonate or esters of malonic acid and malonate. The production of malonic acid, malonate, or esters of malonic acid and malonate can be accomplished at a pH in a range of from about 2 to about 7, about 2.5 to about 4, about 3.5 to about 6, less than, equal to, or greater than about 2, 2.5, 3, 3.5, 4, 4.5, 5, 5. 5, 6, 6.5, or about 7. According to various embodiments, it is possible to use a relatively high pH to increase production rates, but the disclosure is not so limited. The production of malonic acid by yeasts can be measured according to the method of Example 5 with the change that the composition of the selection plate may need to be adapted according to the particular yeast being tested. The production of malonic acid by bacteria can be measured according to the method of Example 5 with the following changes to suit the particular bacterium tested: 1) that the composition of the selection plate 2) the seed medium composition 3) the production medium composition 4) the incubation temperature.

Bacteria can be used to ferment sugars to organic acids. However, bacteria present certain drawbacks for large-scale organic acid production. As organic acids are produced, the fermentation medium becomes increasingly acidic. Lower pH conditions are suitable, because the resultant product is partially or wholly in the acid form. However, most bacteria that produce organic acids do not perform well in strongly acidic environments, and therefore either die or begin producing so slowly that they become economically unviable as the medium becomes more acidic. To prevent this, it becomes necessary to buffer the medium to maintain a higher pH. However, this makes recovery of the organic acid product more difficult and expensive.

There has been increasing interest in recent years around the use of a fungus such as a yeast to ferment sugars to organic acids. Yeasts are used as biocatalysts in a number of industrial fermentations (e.g., batch or fed batch), and present several advantages over bacteria. While many bacteria are unable to synthesize certain amino acids or proteins that they need to grow and metabolize sugars efficiently, most yeast species can synthesize their necessary amino acids or proteins from inorganic nitrogen compounds. Yeasts are also not susceptible to bacteriophage infection, which can lead to loss of productivity or of whole fermentation runs in bacteria.

Although yeasts are attractive candidates for organic acid production, they present several difficulties. First, pathway engineering in yeast can be more difficult than in bacteria. Enzymes in yeast are compartmentalized in the cytoplasm, mitochondria, or peroxisomes, whereas in bacteria they are pooled in the cytoplasm. This means that targeting signals may need to be removed to ensure that all the enzymes of the biosynthetic pathway co-exist in the same compartment within a single cell. Control of transport of pathway intermediates between the compartments may also be necessary to maximize carbon flow to the desired product. Second, not all yeast species meet the necessary criteria for economic fermentation on a large scale. In fact, only a small percentage of yeasts possess the combination of sufficiently high volumetric and specific sugar utilization with the ability to grow robustly under low pH conditions.

Although many yeast species naturally ferment hexose sugars to ethanol, few if any naturally produce significant yields of organic acids. This has led to efforts to genetically modify various yeast species to produce organic acids. Genetically modified yeast strains that produce lactic acid have been previously developed by disrupting or deleting the native pyruvate decarboxylase (PDC) gene and inserting a lactate dehydrogenase (LDH) gene to eliminate ethanol production. This alteration diverts sugar metabolism from ethanol production to lactic acid production. The fermentation products and pathways for yeast differ from those of bacteria, and thus different engineering approaches are necessary to maximize yield. Other native products that may require elimination or reduction in order to enhance organic acid product yield or purity are glycerol, acetate, and diols.

Unlike lactic acid, an organic acid such as malonic acid or a derivative such as malonate and esters of malonic acid is not a major end product of any pathway known in nature, being found in only trace amounts in some bacteria and fungi. Thus, a greater deal of genetic engineering is necessary to generate yeast that produce malonic acid, malonate, esters of malonic acid, or mixtures thereof.

Provided herein are genetically modified yeast cells for the production of organic acids and their derivatives such as malonate, and esters of malonic acid and malonate, methods of making these yeast cells, and methods of using these cells to produce organic acids and their derivatives such as their anionic counterparts and esters thereof. Although yeast cells are extensively described as suitable host microorganisms, the teaches herein can also apply to bacteria host microorganisms. Examples of suitable yeast cells include Crabtree-positive yeasts or Crabtree negative yeasts. In some examples, the yeast is a Crabtree negative yeast exclusively. In some examples the yeast can be chosen from Saccharomycescerevisiae, Kluyveromyceslactis, Kluyveromyces marxianus, Yarrowialipolytica, Pichiakudriavzevii (alternatively referred to as Candidakrusei and Issatchenkiaorientalis), Schizosaccharomycespombe, or a mixture thereof. In some examples, the yeast is Pichiakudriavzevii. In some examples the host cell can include a microorganism such as a bacteria. Examples of suitable bacteria include Streptococcus, Lactobacillus, Bacillus, Escherichia, Salmonella, Neisseria, Acetobactor, Arthrobacter, Aspergillus, Bifdobacterium, Corynebacterium, Pseudomonas, or a mixture thereof

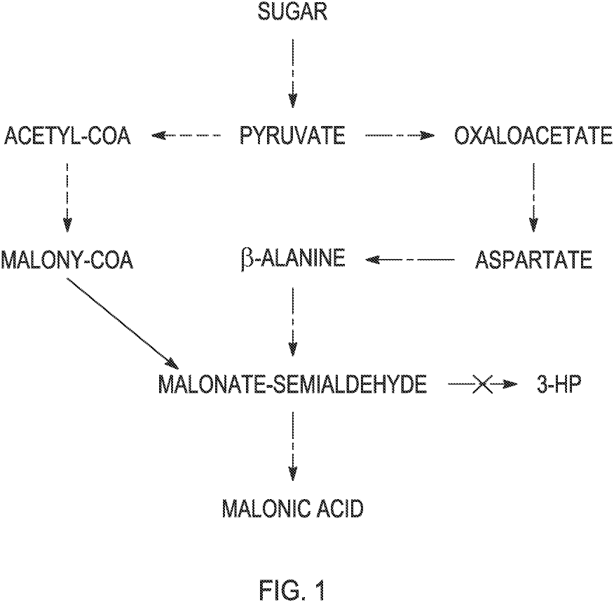

Provided herein in various examples are genetically modified yeast cells having at least one active malonic acid, malonate, esters of malonic acid, or mixtures thereof fermentation pathway from PEP, pyruvate, and/or glycerol to an organic acid and their derivatives such as malonate and esters of malonic acid. An example of a suitable malonic acid, malonate, esters of malonic acid, or mixtures thereof fermentation pathway includes the fermentation pathway set forth in FIG. 1. A yeast cell having a “malonic acid, malonate, esters of malonic acid, and esters thereof fermentation pathway,” refers to a pathway that is capable of producing malonic acid, malonate, esters of malonic acid, or mixtures thereof in measurable yields when cultured under fermentation conditions in the presence of at least one fermentable sugar. Moreover, a “malonic acid, malonate, and esters of malonic acid fermentation pathway,” refers to a pathway that produces one or more enzymes necessary to catalyze the reactions necessary to produce malonic acid, malonate, or mixtures thereof. In some examples, the “malonic acid, malonate, and esters of malonic acid fermentation pathway” can further produce active enzymes necessary to produce one or more enzymes necessary to catalyze the reactions that produce esters of malonic acid or malonate.

A yeast cell having an active malonic acid, malonate, and esters of malonic acid fermentation pathway can include one or more malonic acid, malonate, and esters of malonic acid pathway genes. A “malonic acid, malonate, and esters of malonic acid pathway gene” as used herein refers to the coding region of a nucleotide sequence that encodes an enzyme involved in a malonic acid, malonate, and esters of malonic acid fermentation pathway.

In various examples, the yeast cells provided herein have an active malonic acid, malonate, and esters of malonic acid fermentation pathway that proceeds through PEP or pyruvate, OAA, aspartate, β-alanine, and malonate-semialdehyde intermediates. In these embodiments, the yeast cells comprise a set of malonic acid, malonate, and esters of malonic acid fermentation pathway genes comprising one or more of pyruvate carboxylase (PYC), PEP carboxylase (PPC), aspartate aminotransferase (AAT), aspartate 1-decarboxylase (ADC), p-alanine aminotransferase (BAAT. The malonic acid, malonate, and esters of malonic acid fermentation pathway genes may also include a PEP carboxykinase (PCK.) gene that has been modified to produce a polypeptide that catalyzes the conversion of PEP to OAA (native PCK genes generally produce a polypeptide that catalyzes the reverse reaction of OAAto PEP).

In various examples, the yeast cells provided herein have an active malonic acid, malonate, and esters of malonic acid fermentation pathway that proceeds through pyruvate, acetyl-CoA, malonyl-CoA, and malonate-semialdehyde intermediates. In these embodiments, the yeast cells comprise a set of malonic acid, malonate, and esters of malonic acid fermentation pathway genes comprising one or more of pyruvate dehydrogenase (PDH), acetyl-CoA carboxylase (ACC), malonyl-CoA reductase, CoA acylating malonate-semialdehyde dehydrogenase, 3-HPDH, HIBADH, and 4-hydroxybutyrate.

The malonic acid, malonate, and esters of malonic acid fermentation pathway genes in the yeast cells provided herein may be endogenous or heterologous. “Endogenous” as used herein refers to a genetic material such as a gene, a promoter and a terminator is “endogenous” to a cell if it is (i) native to the cell, (ii) present at the same location as that genetic material is present in the wild-type cell and (iii) under the regulatory control of its native promoter and its native terminator. The term “heterologous” refers to a molecule (e.g., polypeptide or nucleic acid) or activity that is from a source that is different than the referenced organism or, where present, a referenced molecule. Accordingly, a gene or protein that is heterologous to a referenced organism is a gene or protein not found in the native form of that organism. For example, a specific glucoamylase (GA) gene found in a first fungal species and exogenously introduced into a second fungal species that is the host organism is “heterologous” to the second fungal organism. As another example, a specific glucoamylase gene from a fungal species that is modified from its native form with one or more nucleotide changes that affect the function of the gene is “heterologous”. An exogenous nucleic acid can be introduced into the host organism by well-known techniques and can be maintained external to the hosts chromosomal material (e.g., maintained on a non-integrating vector), or can be integrated into the host’s chromosome, such as by a recombination event. An exogenous nucleic acid can encode an enzyme, or portion thereof, that is either homologous or heterologous to the host organism. All heterologous nucleic acids are also exogenous. For purposes of this application, genetic material such as genes, promoters and terminators is “exogenous” to a cell if it is (i) non-native to the cell and/or (ii) is native to the cell, but is present at a location different than where that genetic material is present in the wild-type cell and/or (iii) is under the regulatory control of a non-native promoter and/or non-native terminator. Extra copies of native genetic material are considered as “exogenous” for purposes of this invention, even if such extra copies are present at the same locus as that genetic material is present in the wild-type host strain. “Native” as used herein with regard to a metabolic pathway refers to a metabolic pathway that exists and is active in the wild-type host strain. Genetic material such as genes, promoters and terminators is “native” for purposes of this application if the genetic material has a sequence identical to (apart from individual-to-individual mutations which do not affect function) a genetic component that is present in the genome of the wild-type host cell (i.e., the exogenous genetic component is identical to an endogenous genetic component).”

An exogenous genetic component may have either a native or non-native sequence. An exogenous genetic component with a native sequence comprises a sequence identical to a genetic component that is present in the genome of a native cell (e.g., the exogenous genetic component is identical to an endogenous genetic component). However, the exogenous component is present at a different location in the host cell genome than the endogenous component. For example, an exogenous PYC gene that is identical to an endogenous PYC gene may be inserted into a yeast cell, resulting in a modified cell with a non-native (increased) number of PYC gene copies. An exogenous genetic component with a non-native sequence comprises a sequence that is not found in the genome of a native cell. For example, an exogenous PYC gene from a particular species may be inserted into a yeast cell of another species. An exogenous gene is integrated into the host cell genome in a functional manner, meaning that it is capable of producing an active protein in the host cell. However, in various examples the exogenous gene may be introduced into the cell as part of a vector that is stably maintained in the host cytoplasm. In other examples the exogenous genetic component can be in a native location but can have a modification to its promoter or terminator.

In various examples, the yeast cells provided herein comprise one or more heterologous malonic acid, malonate, and esters of malonic acid fermentation pathway genes. In various examples, the genetically modified yeast cells disclosed herein comprise a single heterologous gene. In other embodiments, the yeast cells comprise multiple heterologous genes. In these embodiments, the yeast cells may comprise multiple copies of a single heterologous gene and/or copies of two or more different heterologous genes. Yeast cells comprising multiple heterologous genes may comprise any number of heterologous genes. For example, these yeast cells may comprise 1 to 20 heterologous genes, and in various examples they may comprise 1 to 7 heterologous genes. Multiple copies of a heterologous gene may be integrated at a single locus such that they are adjacent to one another. Alternatively, they may be integrated at several loci within the host cell’s genome.

In various examples, the yeast cells provided herein include one or more exogenous malonic acid, malonate, and esters of malonic acid fermentation pathway genes. In various examples, the genetically modified yeast cells disclosed herein comprise a single exogenous gene. In other embodiments, the yeast cells comprise multiple exogenous genes. In these embodiments, the yeast cells may comprise multiple copies of a single exogenous gene and/or copies of two or more different exogenous genes. Yeast cells comprising multiple exogenous genes may comprise any number of exogenous genes. For example, these yeast cells may comprise 1 to 20 exogenous genes, and in various examples they may comprise 1 to 7 exogenous genes. Multiple copies of an exogenous gene may be integrated at a single locus such that they are adjacent to one another. Alternatively, they may be integrated at several loci within the host cell’s genome.

In various examples, the yeast cells provided herein comprise one or more endogenous malonic acid, malonate, and esters of malonic acid fermentation pathway genes. In certain of these embodiments, the cells may be engineered to overexpress one or more of these endogenous genes, meaning that the modified cells express the endogenous gene at a higher level than a native cell under at least some conditions. In certain of these embodiments, the endogenous gene being overexpressed may be operatively linked to one or more exogenous regulatory elements. For example, one or more exogenous strong promoters may be introduced into a cell such that they are operatively linked to one or more endogenous malonic acid, malonate, and esters of malonic acid pathway genes.

Malonic acid, malonate, and esters of malonic acid fermentation pathway genes in the modified yeast cells provided herein may be operatively linked to one or more regulatory elements such as a promoter or terminator. As used herein, the term “promoter” refers to an untranslated sequence located upstream (e.g., 5’) to the translation start codon of a gene (generally within about 1 to 1000 base pairs (bp), within about 1 to 500 bp) which controls the start of transcription of the gene. The term “terminator” as used herein refers to an untranslated sequence located downstream (e.g., 3’) to the translation finish codon of a gene (generally within about 1 to 1000 bp, within about 1 to 500 bp, and especially within about 1 to 100 bp) which controls the end of transcription of the gene. A promoter or terminator is “operatively linked” to a gene if its position in the genome relative to that of the gene is such that the promoter or terminator, as the case may be, performs its transcriptional control function. Suitable promoters and terminators are described, for example, in WO99/14335, WO00/71738, WO02/42471, WO03/102201, WO03/102152 and WO03/049525 (all incorporated by reference herein in their entirety).

Regulatory elements linked to malonic acid, malonate, and esters of malonic acid fermentation pathway genes in the cells provided herein may be endogenous, exogenous or heterologous. For example, an exogenous malonic acid, malonate, and esters of malonic acid fermentation pathway gene may be inserted into a yeast cell such that it is under the transcriptional control of an endogenous promoter and/or terminator. Alternatively, the exogenous malonic acid, malonate, and esters of malonic acid fermentation pathway gene may be linked to one or more exogenous regulatory elements. For example, an exogenous gene may be introduced into the cell as part of a gene expression construct that comprises one or more exogenous regulatory elements. In various examples, exogenous regulatory elements, or at least the functional portions of exogenous regulatory elements, may comprise native sequences. In other embodiments, exogenous regulatory elements may comprise non-native sequences. In these embodiments, the exogenous regulatory elements may comprise a sequence with a relatively high degree of sequence identity to a native regulatory element. For example, an exogenous gene may be linked to an exogenous promoter or terminator having at least 50%, at least 60%, at least 70%, at least 80%, or at least 90% sequence identity to a native promoter or terminator. Sequence identity percentages for nucleotide or amino acid sequences can be calculated by methods known in the art, such as for example using BLAST (National Center for Biological Information (NCBI) Basic Local Alignment Search Tool) version 2.2.1 software with default parameters. For example, a sequences having an identity score of at least 90%, using the BLAST version 2.2.1 algorithm with default parameters is considered to have at least 90% sequence identity. The BLAST software is available from the NCBI, Bethesda, Md.

The determination of “corresponding” amino acids from two or more glucoamylases can be determined by alignments of all or portions of their amino acid sequences. Sequence alignment and generation of sequence identity include global alignments and local alignments, which typically use computational approaches. In order to provide global alignment, global optimization forcing sequence alignment spanning the entire length of all query sequences is used. By comparison, in local alignment, shorter regions of similarity within long sequences are identified.

As used herein, an “equivalent position” means a position that is common to the two sequences (e.g., a template GA sequence and a GA sequence having the desired substitution(s)) that is based on an alignment of the amino acid sequences of one glucoamylase or as alignment of the three-dimensional structures. Thus either sequence alignment or structural alignment, or both, may be used to determine equivalence.

In some modes of practice, the BLAST algorithm is used to compare and determine sequence similarity or identity. In addition, the presence or significance of gaps in the sequence which can be assigned a weight or score can be determined. These algorithms can also be used for determining nucleotide sequence similarity or identity. Parameters to determine relatedness are computed based on art known methods for calculating statistical similarity and the significance of the match determined. Gene products that are related are expected to have a high similarity, such as greater than 50% sequence identity. Exemplary parameters for determining relatedness of two or more sequences using the BLAST algorithm can be as follows.

Inspection of nucleic acid or amino acid sequences for two nucleic acids or two polypeptides will reveal sequence identity and similarities between the compared sequences. Sequence alignment and generation of sequence identity include global alignments and local alignments which are carried out using computational approaches. An alignment can be performed using BLAST (National Center for Biological Information (NCBI) Basic Local Alignment Search Tool) version 2.2.31 software with default parameters. Amino acid % sequence identity between amino acid sequences can be determined using standard protein BLAST with the following default parameters: Max target sequences: 100; Short queries: Automatically adjust parameters for short input sequences; Expect threshold: 10; Word size: 6; Max matches in a query range: 0; Matrix: BLOSUM62; Gap Costs: (Existence: 11, Extension: 1); Compositional adjustments: Conditional compositional score matrix adjustment; Filter: none selected; Mask: none selected. Nucleic acid % sequence identity between nucleic acid sequences can be determined using standard nucleotide BLAST with the following default parameters: Max target sequences: 100; Short queries: Automatically adjust parameters for short input sequences; Expect threshold: 10; Word size: 28; Max matches in a query range: 0; MMatch/Mismatch Scores: 1, -2; Gap costs: Linear; Filter: Low complexity regions; Mask: Mask for lookup table only. A sequence having an identity score of XX% (for example, 80%) with regard to a reference sequence using the NCBI BLAST version 2.2.31 algorithm with default parameters is considered to be at least XX% identical or, equivalently, have XX% sequence identity to the reference sequence.

In certain aspects, a regulatory element (e.g., a promoter) linked to a malonic acid, malonate, and esters of malonic acid fermentation pathway gene in the cells provided herein may be foreign to the pathway gene. A regulatory element that is foreign to a pathway gene is a regulatory element that is not linked to the gene in its natural form. A regulatory element foreign to a pathway gene can be native or heterologous, depending on the pathway gene and its relation to the yeast cell. In some instances, a native malonic acid, malonate, and esters of malonic acid fermentation pathway gene is operatively linked to a regulatory element (e.g., a promoter) that is foreign to the pathway gene. In other instances, a heterologous malonic acid, malonate, and esters of malonic acid fermentation pathway gene is operatively linked to an exogenous regulatory element (e.g., a promoter) that is foreign to the pathway gene.

In those embodiments wherein multiple exogenous genes are inserted into a host cell, each exogenous gene may be under the control of a different regulatory element, or two or more exogenous genes may be under the control of the same regulatory elements. For example, where a first exogenous gene is linked to a first regulatory element, a second exogenous gene may also be linked to the first regulatory element, or it may be linked to a second regulatory element. The first and second regulatory elements may be identical or share a high degree of sequence identity, or they be wholly unrelated.

Examples of promoters that may be linked to one or more malonic acid, malonate, and esters of malonic acid fermentation pathway genes in the yeast cells provided herein include, but are not limited to, promoters for PDC1, phosphoglycerate kinase (PGK), xylose reductase (XR), xylitol dehydrogenase (XDH), L-(+)-lactate-cytochrome c oxidoreductase (CYB2), translation elongation factor-1 (TEF1), translation elongation factor-2 (TEF2), enolase (ENO1), glyceraldehyde-3-phosphate dehydrogenase (GAPDH), and orotidine 5’-phosphate decarboxylase (URA3) genes. In these examples, the malonic acid, malonate, and esters of malonic acid fermentation pathway genes may be linked to native, exogenous or heterologous promoters for PDC1, PGK, XR, XDH, CYB2, TEF1, TEF2, ENO1, GAPDH, or URA3 genes. Where the promoters are exogenous, they may be identical to or share a high degree of sequence identity (e.g., at least about 80%, at least about 85%, at least about 90%, at least about 95%, or at least about 99%) with native promoters for PDC1, PGK, XR, XDH, CYB2, TEF1, TEF2, ENO1, GAPDH, or URA3 genes.

Examples of terminators that may be linked to one or more malonic acid, malonate, and esters of malonic acid fermentation pathway genes in the yeast cells provided herein include, but are not limited to, terminators for PDC1, XR, XDH, transaldolase (TAL), transketolase (TKL), ribose 5-phosphate ketol-isomerase (RKI), CYB2, or iso-2-cytochrome c (CYC) genes or the galactose family of genes (especially the GAL10 terminator). In these examples, the malonic acid, malonate, and esters of malonic acid fermentation pathway genes may be linked to native, exogenous or heterologous terminators for PDC1, XR, XDH, TAL, TKL, RKI, CYB2, ENO1, TDH3, TEF1, TEF2, or CYC genes or galactose family genes. Where the terminators are exogenous, they may be identical to or share a high degree of sequence identity (e.g., at least about 80%, at least about 85%, at least about 90%, at least about 95%, or at least about 99%) with native terminators for PDC1, XR, XDH, TAL, TKL, RKI, CYB2, ENO1, TDH3, TEF1, TEF2, or CYC genes or galactose family genes. In various examples, malonic acid, malonate, and esters of malonic acid fermentation pathway genes are linked to a terminator that comprises a functional portion of a native GAL10 gene native to the host cell or a sequence that shares at least 80%, at least 85%, at least 90%, or at least 95% sequence identity with a native GAL10 terminator.

Exogenous genes may be inserted into a yeast host cell via any method known in the art. In various embodiments, the genes are integrated into the host cell genome. Exogenous genes may be integrated into the genome in a targeted or a random manner. In those embodiments where the gene is integrated in a targeted manner, it may be integrated into the loci for a particular gene, such that integration of the exogenous gene is coupled to deletion or disruption of a native gene. For example, introduction of an exogenous malonic acid, malonate, and esters of malonic acid pathway gene may be coupled to deletion or disruption of one or more genes encoding enzymes involved in other fermentation product pathways. Alternatively, the exogenous gene may be integrated into a portion of the genome that does not correspond to a gene.

Targeted integration and/or deletion may utilize an integration construct. The term “construct” as used herein refers to a DNA sequence that is used to transform a host cell. The construct may be, for example, a circular plasmid or vector, a portion of a circular plasmid or vector (such as a restriction enzyme digestion product), a linearized plasmid or vector, or a PCR product prepared using a plasmid or genomic DNA as a template. Methods for transforming a yeast cell with an exogenous construct are described in, for example, WO99/14335, WO00/71738, WO02/42471,WO03/102201, WO03/102152, and WO03/049525. An integration construct can be assembled using two cloned target DNA sequences from an insertion site target. The two target DNA sequences may be contiguous or non-contiguous in the native host genome. In this context, “non-contiguous” means that the DNA sequences are not immediately adjacent to one another in the native genome, but instead are separated by a region that is to be deleted. “Contiguous” sequences as used herein are directly adjacent to one another in the native genome. Where targeted integration is to be coupled to deletion or disruption of a target gene, the integration construct may also be referred to as a deletion construct. In a deletion construct, one of the target sequences may include a region 5’ to the promoter of the target gene, all or a portion of the promoter region, all or a portion of the target gene coding sequence, or some combination thereof. The other target sequence may include a region 3’ to the terminator of the target gene, all or a portion of the terminator region, and/or all or a portion of the target gene coding sequence. Where targeted integration is not to be coupled to deletion or disruption of a native gene, the target sequences are selected such that insertion of an intervening sequence will not disrupt native gene expression. An integration or deletion construct is prepared such that the two target sequences are oriented in the same direction in relation to one another as they natively appear in the genome of the host cell. Where an integration or deletion construct is used to introduce an exogenous gene into a host cell, a gene expression cassette is cloned into the construct between the two target gene sequences to allow for expression of the exogenous gene. The gene expression cassette contains the exogenous gene, and may further include one or more regulatory sequences such as promoters or terminators operatively linked to the exogenous gene. Deletion constructs can also be constructed that do not contain a gene expression cassette. Such constructs are designed to delete or disrupt a gene sequence without the insertion of an exogenous gene.

An integration or deletion construct may comprise one or more selection marker cassettes cloned into the construct between the two target gene sequences. The selection marker cassette contains at least one selection marker gene that allows for selection of transformants. A “selection marker gene” is a gene that encodes a protein needed for the survival and/or growth of the transformed cell in a selective culture medium, and therefore can be used to apply selection pressure to the cell. Successful transformants will contain the selection marker gene, which imparts to the successfully transformed cell at least one characteristic that provides a basis for selection. Typical selection marker genes encode proteins that (a) confer resistance to antibiotics or other toxins (e.g., resistance to bleomycin or zeomycin (e.g., Streptoalloteichushindustanus ble gene), aminoglycosides such as G418 or kanamycin (e.g., kanamycin resistance gene from transposon Tn903), or hygromycin (e.g., aminoglycoside antibiotic resistance gene from E.coli)), (b) complement auxotrophic deficiencies of the cell (e.g., deficiencies in leucine (e.g., K. marxianus LEU2 gene), uracil (e.g., K. marxianus, S.cerevisiae, or I.orientalis URA3 gene), or tryptophan (e.g., K. marxianus, S.cerevisiae, or I.orientalis TRP gene)), (c) enable the cell to synthesize critical nutrients not available from simple media, or (d) confer the ability for the cell to grow on a particular carbon source (e.g., MEL5 gene from S.cerevisiae, which encodes the alpha-galactosidase (melibiase) enzyme and confers the ability to grow on melibiose as the sole carbon source). Various selection markers include the URA3 gene, zeocin resistance gene, G418 resistance gene, MEL5 gene, and hygromycin resistance gene. Another selection marker is an L-lactate:ferricytochrome c oxidoreductase (CYB2) gene cassette, provided that the host cell either natively lacks such a gene or that its native CYB2 gene(s) are first deleted or disrupted. A selection marker gene is operatively linked to one or more promoter and/or terminator sequences that are operable in the host cell. In various examples, these promoter and/or terminator sequences are exogenous promoter and/or terminator sequences that are included in the selection marker cassette. Suitable promoters and terminators are as described herein.

An integration or deletion construct is used to transform the host cell. Transformation may be accomplished using, for example, electroporation and/or chemical transformation (e.g., calcium chloride, lithium acetate-based, etc.) methods. Selection or screening based on the presence or absence of the selection marker may be performed to identify successful transformants. In successful transformants, homologous recombination events at the locus of the target site results in the disruption or the deletion of the target site sequence. Where the construct targets a native gene for deletion or disruption, all or a portion of the native target gene, its promoter, and/or its terminator may be deleted during this recombination event. The expression cassette, selection marker cassette, and any other genetic material between the target sequences in the integration construct is inserted into the host genome at the locus corresponding to the target sequences. Analysis by PCR or Southern analysis can be performed to confirm that the desired insertion/deletion has taken place.

In some embodiments, cell transformation may be performed using DNA from two or more constructs, PCR products, or a combination thereof, rather than a single construct or PCR product. In these embodiments, the 3’ end of one integration fragment overlaps with the 5’ end of another integration fragment. In one example, one construct will contain the first sequence from the locus of the target sequence and a non-functional part of the marker gene cassette, while the other will contain the second sequence from the locus of the target sequence and a second non-functional part of the marker gene cassette. The parts of the marker gene cassette are selected such that they can be combined to form a complete cassette. The cell is transformed with these pieces simultaneously, resulting in the formation of a complete, functional marker or structural gene cassette. Successful transformants can be selected for on the basis of the characteristic imparted by the selection marker. In another example, the selection marker resides on one fragment but the target sequences are on separate fragments, so that the integration fragments have a high probability of integrating at the site of interest. In other embodiments, transformation from three linear DNAs can be used to integrate exogenous genetic material. In these embodiments, one fragment overlaps on the 5’ end with a second fragment and on the 3’ end with a third fragment.

An integration or deletion construct may be designed such that the selection marker gene and some or all of its regulatory elements can become spontaneously deleted as a result of a subsequent homologous recombination event. A convenient way of accomplishing this is to design the construct such that the selection marker gene and/or regulatory elements are flanked by repeat sequences. Repeat sequences are identical DNA sequences, native or non-native to the host cell, and oriented on the construct in the same or opposite direction with respect to one another. The repeat sequences are advantageously about 50 to 1500 bp in length, and do not have to encode for anything. Inclusion of the repeat sequences permits a homologous recombination event to occur, which results in deletion of the selection marker gene and one of the repeat sequences. Since homologous recombination occurs with relatively low frequency, it may be necessary to grow transformants for several rounds on nonselective media to allow for the spontaneous homologous recombination to occur in some of the cells. Cells in which the selection marker gene has become spontaneously deleted can be selected or screened on the basis of their loss of the selection characteristic imparted by the selection marker gene. In certain cases, expression of a recombinase enzyme may enhance recombination between the repeated sites.

An exogenous malonic acid, malonate, and esters of malonic acid fermentation pathway gene in the modified yeast cells provided herein may be derived from a source gene from any suitable source. For example, an exogenous gene may be derived from a yeast, fungal, bacterial, plant, insect, or mammalian source. As used herein, an exogenous gene that is “derived from” a native source gene encodes a polypeptide that 1) is identical to a polypeptide encoded by the native gene, 2) shares at least 50%, at least 60%, at least 70%, at least 80%, at least 85%, at least 90%, at least 95%, at least 97%, or at least 99% sequence identity with a polypeptide encoded by the native gene, and/or 3) has the same function in a malonic acid, malonate, and esters of malonic acid pathway as the polypeptide encoded by the native gene. For example, a malonic acid, malonate, and esters of malonic acid fermentation pathway gene that is derived from a Danausplexippus malonic acid, malonate, and esters of malonic acid fermentation pathway gene may encode a polypeptide comprising the amino acid sequence of SEQ ID NO: 36, a polypeptide with at least 50%, at least 60%, at least 70%, at least 80%, at least 85%, at least 90%, at least 95%, at least 97%, or at least 99% sequence identity to the amino acid sequence of SEQ ID NO: 36, and/or a polypeptide that has the ability to catalyze the conversion of aspartate to beta-alanine. A gene derived from a native gene may comprise a nucleotide sequence with at least 50%, at least 60%, at least 70%, at least 80%, at least 85%, at least 90%, at least 95%, at least 97%, or at least 99% sequence identity to the coding region of the native gene. In various examples, a gene derived from a native gene may comprise a nucleotide sequence that is identical to the coding region of the source gene. For example, a malonic acid, malonate, and esters of malonic acid dehydrogenase gene that is derived from a Danausplexippus ADC gene may comprise the nucleotide sequence of SEQ ID NO: 51 or a nucleotide sequence with at least 50%, at least 60%, at least 70%, at least 80%, at least 85%, at least 90%, at least 95%, at least 97%, or at least 99% sequence identity to the nucleotide sequence of SEQ ID NO: 51.

In various examples of the modified yeast cells provided herein, the native source gene from which the exogenous malonic acid, malonate, and esters of malonic acid fermentation pathway gene that is derived produces a polypeptide that is involved in a malonic acid, malonate, and esters of malonic acid fermentation pathway. In other embodiments, however, the native source gene may encode a polypeptide that is not involved in a malonic acid, malonate, and esters of malonic acid fermentation pathway or that catalyzes a reverse reaction in a malonic acid, malonate, and esters of malonic acid fermentation pathway. In these embodiments, the exogenous malonic acid, malonate, and esters of malonic acid pathway gene will have undergone one or more targeted or random mutations versus the native source gene that result in modified activity and/or substrate preference. For example, a native source gene may be mutated to generate a gene that encodes a polypeptide with increased activity in a desired reaction direction and/or decreased activity in a non-desired direction in a malonic acid, malonate, and esters of malonic acid fermentation pathway. For example, where the native source gene encodes a polypeptide capable of catalyzing both a forward and reverse reactions in a malonic acid, malonate, and esters of malonic acid fermentation pathway, the gene may be modified such that the resultant exogenous gene has increased activity in the forward direction and decreased activity in the reverse direction. Similarly, a native source gene may be mutated to produce a gene that encodes a polypeptide with different substrate preference than the native polypeptide. For example, a malonic acid, malonate, and esters of malonic acid pathway gene may be mutated to produce a polypeptide with the ability to act on a substrate that is either not preferred or not acted on at all by the native polypeptide. In these embodiments, the polypeptide encoded by the exogenous malonic acid, malonate, and esters of malonic acid pathway gene may catalyze a reaction that the polypeptide encoded by the native source gene is completely incapable of catalyzing. A native source gene may also be mutated such that the resultant malonic acid, malonate, and esters of malonic acid pathway gene exhibits decreased feedback inhibition at the DNA, RNA, or protein level in the presence of one or more downstream malonic acid, malonate, and esters of malonic acid pathway intermediates or side products.

In various examples of the modified yeast cells provided herein, an exogenous malonic acid, malonate, and esters of malonic acid pathway gene may be derived from the host yeast species. For example, where the host cell is Saccharomycescerevisiae, an exogenous gene may be derived from an Saccharomycescerevisiae gene. In these embodiments, the exogenous gene may comprise a nucleotide sequence identical to the coding region of the native gene, such that incorporation of the exogenous gene into the host cell increases the copy number of a native gene sequence and/or changes the regulation or expression level of the gene if under the control of a promoter that is different from the promoter that drives expression of the gene in a wild-type cell. In other embodiments, the exogenous malonic acid, malonate, and esters of malonic acid pathway gene may comprise a nucleotide sequence that differs from the coding region of a native malonic acid, malonate, and esters of malonic acid pathway gene, but nonetheless encodes a polypeptide that is identical to the polypeptide encoded by the native malonic acid, malonate, and esters of malonic acid pathway gene. In still other embodiments, the exogenous malonic acid, malonate, and esters of malonic acid pathway gene may comprise a nucleotide sequence that encodes a polypeptide with at least 50%, at least 60%, at least 70%, at least 80%, at least 85%, at least 90%, at least 95%, at least 97%, or at least 99% sequence identity to a polypeptide encoded by one or more native malonic acid, malonate, and esters of malonic acid pathway genes. In certain of these embodiments, the exogenous gene comprises a nucleotide sequence with at least 50%, at least 60%, at least 70%, at least 80%, at least 85%, at least 90%, at least 95%, at least 97%, or at least 99% sequence identity to one or more native genes. In still other embodiments, the exogenous malonic acid, malonate, and esters of malonic acid gene may encode a polypeptide that has less than 50% sequence identity to a polypeptide encoded by a native malonic acid, malonate, and esters of malonic acid pathway gene but which nonetheless has the same function as the native polypeptide in a malonic acid, malonate, and esters of malonic acid fermentation pathway (e.g., the ability to catalyze the same reaction). A native source gene may be subjected to mutagenesis if necessary to provide a coding sequence starting with the usual eukaryotic starting codon (ATG), or for other purposes.

In other embodiments, the exogenous malonic acid, malonate, and esters of malonic acid pathway gene may be derived from a species that is different than that of the host yeast cell. In certain of these embodiments, the exogenous malonic acid, malonate, and esters of malonic acid pathway gene may be derived from a different yeast species than the host cell. For example, where the host cell is Saccharomycescerevisiae. In other embodiments, the exogenous malonic acid, malonate, and esters of malonic acid pathway gene may be derived from a fungal, bacterial, plant, insect, or mammalian source. For example, where the host cell is Saccharomycescerevisiae, the exogenous gene may be derived from a bacterial source such as E.coli. In those embodiments where the exogenous malonic acid, malonate, and esters of malonic acid pathway gene is derived from a non-yeast source, the exogenous gene sequence may be codon-optimized for expression in a yeast host cell.

In those embodiments where the exogenous malonic acid, malonate, and esters of malonic acid pathway gene is derived from a species other than the host cell species, the exogenous gene may encode a polypeptide identical to a polypeptide encoded by a native malonic acid, malonate, and esters of malonic acid pathway gene from the source organism. In certain of these embodiments, the exogenous malonic acid, malonate, and esters of malonic acid pathway gene may be identical to a native malonic acid, malonate, and esters of malonic acid pathway gene from the source organism. In other embodiments, the exogenous gene may share at least 50%, at least 60%, at least 70%, at least 80%, at least 85%, at least 90%, at least 95%, at least 97%, or at least 99% sequence identity to a native malonic acid, malonate, and esters of malonic acid pathway gene from the source organism. In other embodiments, the exogenous malonic acid, malonate, and esters of malonic acid pathway gene may encode a polypeptide that shares at least 50%, at least 60%, at least 70%, at least 80%, at least 85%, at least 90%, at least 95%, at least 97%, or at least 99% sequence identity with a polypeptide encoded by a native malonic acid, malonate, and esters of malonic acid pathway gene from the source organism. In certain of these embodiments, the exogenous gene may comprise a nucleotide sequence with at least 50%, at least 60%, at least 70%, at least 80%, at least 85%, at least 90%, at least 95%, at least 97%, or at least 99% sequence identity to one or more native malonic acid, malonate, and esters of malonic acid pathway genes from the source organism. In still other embodiments, the exogenous malonic acid, malonate, and esters of malonic acid gene may encode a polypeptide that has less than 50% sequence identity to a polypeptide encoded by a native malonic acid, malonate, and esters of malonic acid pathway gene from the source organism, but which nonetheless has the same function as the native polypeptide from the source organism in a malonic acid, malonate, and esters of malonic acid fermentation pathway.

In various examples, the yeast cells provided herein express one or more malonic acid, malonate, and esters of malonic acid pathway genes encoding enzymes selected from the group consisting of ACC (catalyzes the conversion of acetyl-CoA to malonyl-CoA), alanine 2,3 aminomutase (AAM, catalyzes the conversion of alanine to β-alanine), alanine dehydrogenase (catalyzes the conversion of pyruvate to alanine), aldehyde dehydrogenase (catalyzes the conversion of 3-HPA to 3-HP), KGD (catalyzes the conversion of OAA to malonate-semialdehyde), AAT (catalyzes the conversion of OAA to aspartate), ADC (catalyzes the conversion of aspartate to β-alanine), BCKA (catalyzes the conversion of OAA to malonate-semialdehyde), BAAT (catalyzes the conversion of β-alanine to malonate-semialdehyde), 4-aminobutyrate aminotransferase (gabT, catalyzes the conversion of P-alanine to malonate-semialdehyde), β-alanyl-CoA ammonia lyase (catalyzes the conversion of β-alanyl-CoA to acrylyl-CoA), Co-A acylating malonate-semialdehyde dehydrogenase (catalyzes the conversion of malonyl-CoA to malonate-semialdehyde), CoA synthetase (catalyzes the conversion of β-alanine to β-alanyl-CoA or the conversion of lactate to lactyl-CoA), CoA transferase (catalyzes the conversion of β-alanine to β-alanyl-CoA and/or the conversion of lactate to lactyl-CoA), glycerol dehydratase (catalyzes the conversion of glycerol to 3-HPA), IPDA (catalyzes the conversion of OAA to malonate-semialdehyde), LDH (catalyzes the conversion of pyruvate to lactate), lactyl-CoA dehydratase (catalyzes the conversion of lactyl-CoA to acrylyl-CoA), malate decarboxylase (catalyzes the conversion of malate to 3-HP), malate dehydrogenase (catalyzes the conversion of OAA to malate), malonyl-CoA reductase (catalyzes the conversion of malonyl-CoA to malonate-semialdehyde or 3-HP), OAA formatelyase (also known as pyruvate-formate lyase and ketoacid formate-lyase, catalyzes the conversion of OAA to malonyl-CoA), OAA dehydrogenase (catalyzes the conversion of OAA to malonyl CoA); PPC (catalyzes the conversion of PEP to OAA), pyruvate/alanine aminotransferase (catalyzes the conversion of pyruvate to alanine), PYC (catalyzes the conversion of pyruvate to OAA), PDH (catalyzes the conversion of pyruvate to acetyl-CoA), 2-keto acid decarboxylase (catalyzes the conversion of OAA to malonate-semialdehyde), 3-HP-CoA dehydratase (also known as acrylyl-CoA hydratase, catalyzes the conversion of acrylyl-CoA to 3-HP-CoA), 3-HPDH (catalyzes the conversion of malonate-semialdehyde to 3-HP), 3-HP-CoA hydrolase (catalyzes the conversion of 3-HP-CoA to 3-HP), HIBADH (catalyzes the conversion of malonate-semialdehyde to 3-HP), 3-hydroxyisobutyryl-CoA hydrolase (catalyzes the conversion of 3-HP-CoA to 3-HP), 4-hydroxybutyrate dehydrogenase (catalyzes the conversion of malonate-semialdehyde to 3-HP), and malonate-semialdehyde dehydrogenase (catalyzes the conversion of malonate-semialdehyde to malonic acid, malonate, or esters of malonic acid). For each of these enzyme activities, the reaction of interest in parentheses may be a result of native or non-native activity.

A “pyruvate carboxylase gene” or “PYC gene” as used herein refers to any gene that encodes a polypeptide with pyruvate carboxylase activity, meaning the ability to catalyze the conversion of pyruvate, CO₂, and ATP to OAA, ADP, and phosphate. In various examples, a PYC gene may be derived from a yeast source.

A “PEP carboxylase gene” or “PPC gene” as used herein refers to any gene that encodes a polypeptide with PEP carboxylase activity, meaning the ability to catalyze the conversion of PEP and CO2 to OAA and phosphate. In various examples, a PPC gene may be derived from a bacterial PPC gene. In certain of these embodiments, the gene may have undergone one or more mutations versus the native gene in order to generate an enzyme with improved characteristics. For example, the gene may have been mutated to encode a PPC polypeptide with increased resistance to aspartate feedback versus the native polypeptide. In other embodiments, the PPC gene may be derived from a plant source.

An “aspartate aminotransferase gene” or “AAT gene” as used herein refers to any gene that encodes a polypeptide with aspartate aminotransferase activity, meaning the ability to catalyze the conversion of OAA to aspartate. Enzymes having aspartate aminotransferase activity are classified as EC 2.6.1.1. In various examples, an AAT gene may be derived from a yeast source such as Saccharomycescerevisiae.

An “aspartate decarboxylase gene” or “ADC gene” or “panD” gene as used herein refers to any gene that encodes a polypeptide with aspartate decarboxylase activity, meaning the ability to catalyze the conversion of aspartate to β-alanine. Enzymes having aspartate decarboxylase activity are classified as EC 4.1.1.11. In various examples, an ADC gene may be derived from a bacterial source. Because an active aspartate decarboxylase may require proteolytic processing of an inactive proenzyme, in these embodiments the yeast host cell should be selected to support formation of an active enzyme coded by a bacterial ADC gene. The panD or ADC genes may be heterologous.

A “β-alanine aminotransferase gene” or “BAAT gene” as used herein refers to any gene that encodes a polypeptide with β-alanine aminotransferase activity, meaning the ability to catalyze the conversion of β-alanine to malonate-semialdehyde. Enzymes having β-alanine aminotransferase activity are classified as EC 2.6.1.19. In various examples, a BAAT gene may be derived from a yeast source. For example, a BAAT gene may be derived from the Saccharomycescerevisiae homolog to the pyd4 gene.

A BAAT gene may also be a “4-aminobutyrate aminotransferase” or “gabT gene” meaning that it has native activity on 4-aminobutyrate as well as β-alanine. Alternatively, a BAAT gene may be derived by random or directed engineering of a native gabT gene from a bacterial or yeast source to encode a polypeptide with BAAT activity. For example, a BAAT gene may be derived from the S.avermitilis gabT.

A “3-HP dehydrogenase gene” or “3-HPDH gene” as used herein refers to any gene that encodes a polypeptide with 3-HP dehydrogenase activity, meaning the ability to catalyze the conversion of malonate-semialdehyde to 3-HP. Enzymes having 3-HP dehydrogenase activity are classified as EC 1.1.1.59 if they utilize an NAD(H) cofactor, and as EC 1.1.1.298 if they utilize an NADP(H) cofactor. Enzymes classified as EC 1.1.1.298 are alternatively referred to as malonate-semialdehyde reductases. In some examples, the microorganism can be free of a 3-HP dehydrogenase gene such that substantially no malonate-semialdehyde is converted to 3-HP. Alternatively, if the 3-HPDH gene is present, is expression can be substantially mitigated such that a minimal or predetermined amount of 3-HP is produced and the majority of the malonate-semialdehyde is instead converted into malonic acid, malonate, esters of malonic acid, or mixtures thereof.

In various examples, a 3-HPDH gene may be derived from a yeast source. For example, a 3-HPDH gene may be derived from the Saccharoniycescerevisiae homolog to the YMR226C gene. In other embodiments, the 3-HPDH gene may be derived from a bacterial source. For example, a 3-HPDH gene may be derived from an E.coli ydfG gene.

A “3-hydroxyisobutyrate dehydrogenase gene” or “HIBADH gene” as used herein refers to any gene that encodes a polypeptide with 3-hydroxyisobutyrate dehydrogenase activity, meaning the ability to catalyze the conversion of 3-hydroxyisobutyrate to methylmalonate-semialdehyde. Enzymes having 3-hydroxyisobutyrate dehydrogenase activity are classified as EC 1.1.1.31. Some 3-hydroxyisobutyrate dehydrogenases also have 3-HPDH activity. In various examples, an HIBADH gene may be derived from a bacterial source. For example, an HIBADH gene may be derived from an A.faecalis M3A gene.

A “4-hydroxybutyrate dehydrogenase gene” as used herein refers to any gene that encodes a polypeptide with 4-hydroxybutyrate dehydrogenase activity, meaning the ability to catalyze the conversion of 4-hydroxybutanoate to succinate-semialdehyde. Enzymes having 4-hydroxybutyrate dehydrogenase activity are classified as EC 1.1.1.61. Some 4-hydroxybutyrate dehydrogenases also have 3-HPDH activity. In various examples, a 4-hydroxybutyrate dehydrogenase gene may be derived from a bacterial source. For example, a 4-hydroxybutyrate dehydrogenase gene may be derived from a R.eutropha H16 4hbd gene.

A “malate dehydrogenase gene” as used herein refers to any gene that encodes a polypeptide with malate dehydrogenase activity, meaning the ability to catalyze the conversion of OAA to malate. In various examples, a malate dehydrogenase gene may be derived from a bacterial or yeast source.

A “malate decarboxylase gene” as used herein refers to any gene that encodes a polypeptide with malate decarboxylase activity, meaning the ability to catalyze the conversion of malate to 3-HP. According various embodiments, little to none of this polypeptide will be present. Malate decarboxylase activity is not known to occur naturally. Therefore, a malate decarboxylase gene may be derived by incorporating one or more mutations into a native source gene that encodes a polypeptide with acetolactate decarboxylase activity. Polypeptides with acetolactate decarboxylase activity catalyze the conversion of 2-hydroxy-2-methyl-3-oxobutanoate to 2-acetoin, and are classified as EC 4.1.1.5. In various examples, a malate decarboxylase gene may be derived from a bacterial source. For example, a malate decarboxylase gene may be derived from an L.lactis aldB

A “branched-chain alpha-keto acid decarboxylase gene” or “BCKA gene” as used herein refers to any gene that encodes a polypeptide with branched-chain alpha-keto acid decarboxylase activity, which can serve to decarboxylate a range of alpha-keto acids from three to six carbons in length. Enzymes having BCKA activity are classified as EC 4.1.1.72. A BCKA gene may be used to derive a gene encoding a polypeptide capable of catalyzing the conversion of OAA to malonate-semialdehyde. This activity may be found in a native BCKA gene, or it may be derived by incorporating one or more mutations into a native BCKA gene. In various examples, a BCKA gene may be derived from a bacterial source. For example, a BCKA gene may be derived from a L. lactis kdcA gene.

An “indolepyruvate decarboxylase gene” or “IPDA gene” as used herein refers to any gene that encodes a polypeptide with indolepyruvate decarboxylase activity, meaning the ability to catalyze the conversion of indolepyruvate to indoleacetaldehyde. Enzymes having IPDA activity are classified as EC 4.1.1.74. An IPDA gene may be used to derive a gene encoding a polypeptide capable of catalyzing the conversion of OAA to malonate-semialdehyde. This activity may be found in a native IPDA gene, or it may be derived by incorporating one or more mutations into a native IPDA gene. In various examples, an indolepyruvate decarboxylase gene may be derived from a yeast, bacterial, or plant source.

A “pyruvate decarboxylase gene” or “PDC gene” as used herein refers to any gene that encodes a polypeptide with pyruvate decarboxylase activity, meaning the ability to catalyze the conversion of pyruvate to acetaldehyde. Enzymes having PDC activity are classified as EC 4.1.1.1. In various embodiments, a PDC gene that is incorporated into a modified yeast cell as provided herein has undergone one or more mutations versus the native gene from which it was derived such that the resultant gene encodes a polypeptide capable of catalyzing the conversion of OAA to malonate-semialdehyde. In various examples, a PDC gene may be derived from a yeast source. According to various embodiments, the engineered microorganism can have reduced pyruvate decarboxylase (PDC) activity compared to a native form of the engineered microorganism. According to some embodiments the engineered microorganism can have zero PDC activity.

An “OAA formatelyase gene” as used herein refers to any gene that encodes a polypeptide with OAA formatelyase activity, meaning the ability to catalyze the conversion of an acylate ketoacid to its corresponding CoA derivative. A polypeptide encoded by an OAA formatelyase gene may have activity on pyruvate or on another ketoacid. In various examples, an OAA formatelyase gene encodes a polypeptide that converts OAA to malonyl-CoA.

A “malonyl-CoA reductase gene” as used herein refers to any gene that encodes a polypeptide with malonyl-CoA reductase activity, meaning the ability to catalyze the conversion of malonyl-CoA to malonate-semialdehyde (also referred to as Co-A acylating malonate-semialdehyde dehydrogenase activity). In various examples, a malonyl-CoA reductase gene may be derived from a bifunctional malonyl-CoA reductase gene which also has the ability to catalyze the conversion of malonate-semialdehyde to 3-HP. According to various embodiments the engineered microorganisms can include little to none of this polypeptide.

A “pyruvate dehydrogenase gene” or “PDH gene” as used herein refers to any gene that encodes a polypeptide with pyruvate dehydrogenase activity, meaning the ability to catalyze the conversion of pyruvate to acetyl-CoA. In various examples, a PDH gene may be derived from a yeast source. For example, a PDH gene may be derived from an S.cerevisiae LAT1, PDA1, PDB1, or LPD gene. In other embodiments, a PDH gene may be derived from a bacterial source. For example, a PDH gene may be derived from an E.coli strain K12 substr. MG1655 aceE, aceF, or lpd gene, respectively, or a B.subtilis pdhA, pdhB, pdhC, or pdhD gene.

An “acetyl-CoA carboxylase gene” or “ACC gene” as used herein refers to any gene that encodes a polypeptide with acetyl-CoA carboxylase activity, meaning the ability to catalyze the conversion of acetyl-CoA to malonyl-CoA. Enzymes having acetyl-CoA carboxylase activity are classified as EC 6.4.1.2. In various examples, an acetyl-CoA carboxylase gene may be derived from a yeast source. For example, an acetyl-CoA carboxylase gene may be derived from an S.cerevisiae ACC1 gene. In other embodiments, an acetyl-CoA carboxylase gene may be derived from a bacterial source. For example, an acetyl-CoA carboxylase gene may be derived from an E.coli accA, accB, accC, or accD gene.

An “alanine dehydrogenase gene” as used herein refers to any gene that encodes a polypeptide with alanine dehydrogenase activity, meaning the ability to catalyze the NAD-dependent reductive amination of pyruvate to alanine. Enzymes having alanine dehydrogenase activity are classified as EC 1.4.1.1. In various examples, an alanine dehydrogenase gene may be derived from a bacterial source. For example, an alanine dehydrogenase gene may be derived from an B.subtilis alanine dehydrogenase gene.

A “pyruvate/alanine aminotransferase gene” as used herein refers to any gene that encodes a polypeptide with pyruvate/alanine aminotransferase activity, meaning the ability to catalyze the conversion of pyruvate and L-glutamate to alanine and 2-oxoglutarate. In various examples, a pyruvate/alanine aminotransferase gene is derived from a yeast source. For example, a pyruvate/alanine aminotransferase gene may be derived from an S.pombe pyruvate/alanine aminotransferase gene.

An “alanine 2,3 aminomutase gene” or “AAM gene” as used herein refers to a gene that encodes a polypeptide with alanine 2,3 aminomutase activity, meaning the ability to catalyze the conversion of alanine to β-alanine. Alanine 2,3 aminomutase activity is not known to occur naturally. Therefore, an alanine 2,3 aminomutase gene can be derived by incorporating one or more mutations into a native source gene that encodes a polypeptide with similar activity such as lysine 2,3 aminomutase activity (see, e.g., U.S. Pat. No. 7,309,597). In various examples, the native source gene may be a B.subtilis lysine 2,3 aminomutase gene, a P.gingivalis lysine 2,3 aminomutase gene, or a F.nucleatum (ATCC-10953) lysine 2,3 aminomutase gene.

A “CoA transferase gene” as used herein refers to any gene that encodes a polypeptide with CoA transferase activity, which in one example includes the ability to catalyze the conversion of β-alanine to β-alanyl-CoA and/or the conversion of lactate to lactyl-CoA. In various examples, a CoA transferase gene may be derived from a yeast source. In other embodiments, a CoA transferase gene may be derived from a bacterial source. For example, a CoA transferase gene may be derived from an M.elsdenii CoA transferase.

A “CoA synthetase gene” as used herein refers to any gene that encodes a polypeptide with CoA synthetase activity. In one example this includes the ability to catalyze the conversion of β-alanine to β-alanyl-CoA. In another example, this includes the ability to catalyze the conversion of lactate to lactyl-CoA. In various examples, a CoA synthetase gene may be derived from a yeast source. For example, a CoA synthetase gene may be derived from an S.cerevisiae CoA synthetase gene. In other embodiments, a CoA synthetase gene may be derived from a bacterial source. For example, a CoA synthetase gene may be derived from an E.coli CoA synthetase, R.sphaeroides, or S.enterica CoA synthetase gene.

A “β-alanyl-CoA ammonia lyase gene” as used herein refers to any gene that encodes a polypeptide with P-alanyl-CoA ammonia lyase activity, meaning the ability to catalyze the conversion of β-alanyl-CoA to acrylyl-CoA. In various examples, a β-alanyl-CoA ammonia lyase gene may be derived from a bacterial source, such as a C.propionicum β-alanyl-CoA ammonia lyase gene.

A “3-HP-CoA dehydratase gene” or “acrylyl-(CoA hydratase gene” as used herein refers to any gene that encodes a polypeptide with 3-HP-CoA dehydratase gene activity, meaning the ability to catalyze the conversion of acrylyl-CoA to 3-HP-CoA. Enzymes having 3-HP-CoA dehydratase activity are classified as EC 4.2.1.116. In various examples, a 3-HP-CoA dehydratase gene may be derived from a yeast or fungal source, such as a P.sojae 3-HP-CoA dehydratase gene. In other embodiments, a 3-HP-CoA dehydratase gene may be derived from a bacterial source. For example, a 3-HP-CoA dehydratase gene may be derived from a C.aurantiacus 3-HP-CoA dehydratase gene, an R.rubrum 3-HP-CoA dehydratase gene, or an R.capsulates 3-HP-CoA dehydratase gene encoding the amino acid sequence. In still other embodiments, a 3-HP-CoA dehydratase gene may be derived from a mammalian source. For example, a 3-HP-CoA dehydratase gene may be derived from a H.sapiens 3-HP-CoA dehydratase gene.

A “3-HP-CoA hydrolase gene” as used herein refers to any gene that encodes a polypeptide with 3-HP-CoA hydrolase activity, meaning the ability to catalyze the conversion of 3-HP-CoAto 3-HP. In various examples, a 3-HP-CoA gene may be derived from a yeast or fungal source. In other embodiments, a 3-HP-CoA gene may be derived from a bacterial or mammalian source.

A “3-hydroxyisobutyryl-CoA hydrolase gene” as used herein refers to any gene that encodes a polypeptide with 3-hydroxyisobutyryl-CoA hydrolase activity, which in one example includes the ability to catalyze the conversion of 3-HP-CoA to 3-HP. In various examples, a 3-hydroxyisobutyryl-CoA hydrolase gene may be derived from a bacterial source, such as a P.fluorescens 3-hydroxyisobutyryl-CoA hydrolase gene or a B.cereus 3-hydroxyisobutyryl-CoA hydrolase gene. In other embodiments, a 3-hydroxyisobutyryl-CoA hydrolase gene may be derived from a mammalian source, such as a H.sapiens 3-hydroxyisobutyryl-CoA hydrolase gene.

A “lactate dehydrogenase gene” or “LDH gene” as used herein refers to any gene that encodes a polypeptide with lactate dehydrogenase activity, meaning the ability to catalyze the conversion of pyruvate to lactate. In various examples, an LDH gene may be derived from a fungal, bacterial, or mammalian source.

A “lactyl-CoA dehydratase gene” as used herein refers to any gene that encodes a polypeptide with lactyl-CoA dehydratase activity, meaning the ability to catalyze the conversion of lactyl-CoA to acrylyl-CoA. In various examples, a lactyl-CoA dehydratase gene may be derived from a bacterial source. For example, a lactyl-CoAdehydratase gene may be derived from an M.elsdenii lactyl-CoA dehydratase E1, EIIa, or EIIb subunit gene.

An “aldehyde dehydrogenase gene” as used herein refers to any gene that encodes a polypeptide with aldehyde dehydrogenase activity, which in one example includes the ability to catalyze the conversion of 3-HPA to 3-HP and vice versa. In various examples, an aldehyde dehydrogenase gene may be derived from a yeast source, such as an S.cerevisiae aldehyde dehydrogenase gene or an Saccharomycescerevisiae aldehyde dehydrogenase gene. In other embodiments, an aldehyde dehydrogenase may be derived from a bacterial source, such as an E.coli aldH gene or a K.pneumoniae aldehyde dehydrogenase gene.

A “glycerol dehydratase gene” as used herein refers to any gene that encodes a polypeptide with glycerol dehydratase activity, meaning the ability to catalyze the conversion of glycerol to 3-HPA. In various examples, a glycerol dehydratase gene may be derived from a bacterial source, such as a K.pneumonia or C.freundii glycerol dehydratase gene.

A “malonate-semialdehyde dehydrogenase gene” as used herein refers to any gene that encodes a polypeptide with malonate-semialdehyde dehydrogenase (MSADh) activity, meaning the ability to catalyze the conversion of malonate-semialdehyde to malonic acid, malonate, esters of malonic acid, or mixtures thereof. In various examples, a malonate-semialdehyde dehydrogenase gene can be derived from a yeast source, such as an S.cerevisiae malonate-semialdehyde dehydrogenase gene. In other embodiments, malonate-semialdehyde dehydrogenase may be derived from a bacterial source, such as an E.coli encoding the amino acid sequence set forth in SEQ ID NO: 7. In some embodiments the malonate-semialdehyde dehydrogenase comprises at least 95% sequence identity to any one of SEQ ID Nos: 7, 9, 11, 13, 15, 17, 19, 21, 23, 27, 29, and 31. In some embodiments, the malonate-semialdehyde dehydrogenase comprises at least 90% sequence identity to SEQ ID No: 11. In some embodiments, the malonate-semialdehyde dehydrogenase comprises at least 95% sequence identity to SEQ ID No: 11. In some embodiments the malonate-semialdehyde dehydrogenase comprises at least 90% sequence identity to any one of SEQ ID Nos: 9, 11, 23, 27, 29, and 31. In some embodiments, the malonate-semialdehyde dehydrogenase comprises at least 95% sequence identity to any one of SEQ ID Nos: 9, 11, 23, 27, 29, and 31. In some embodiments, an amino acid residue of a polypeptide that aligns with amino acid residue 160 of SEQ ID NO: 11 is not phenylalanine. However, in other embodiments, an amino acid residue of a polypeptide that aligns with amino acid residue 160 of SEQ ID NO: 11 is tryptophan. The copy number of the malonate-semialdehyde dehydrogenase gene can be increased over 1X. For example, a copy number of the gene can be 2X, 3X, 4X, 5X, 6X, 7X, 8X, 9X, 10X, or higher. It is suspected that the increase in copy number can lead to a linear increase in the amount of malonic acid or malonate produced.

In various examples, the genetically modified yeast cells provided herein further comprise a deletion or disruption of one or more native genes. “Deletion or disruption” with regard to a native gene means that either the entire coding region of the gene is eliminated (deletion) or the coding region of the gene, its promoter, and/or its terminator region is modified (such as by deletion, insertion, or mutation) such that the gene no longer produces an active enzyme, produces a severely reduced quantity (at least 75% reduction, or at least 90% reduction) of an active enzyme, or produces an enzyme with severely reduced (at least 75% reduced, or at least 90% reduced) activity.

In various examples, deletion or disruption of one or more native genes results in a deletion or disruption of one or more native metabolic pathways. “Deletion or disruption” with regard to a metabolic pathway means that the pathway is either inoperative or else exhibits activity that is reduced by at least 75%, at least 85%, or at least 95% relative to the native pathway. In various examples, deletion or disruption of a native metabolic pathway is accomplished by incorporating one or more genetic modifications that result in decreased expression of one or more native genes that reduce malonic acid, malonate, esters of malonic acid, or mixtures thereof production.

In various examples, deletion or disruption of native gene can be accomplished by forced evolution, mutagenesis, or genetic engineering methods, followed by appropriate selection or screening to identify the desired mutants. In various examples, deletion or disruption of a native host cell gene may be coupled to the incorporation of one or more exogenous genes into the host cell, e.g., the exogenous genes may be incorporated using a gene expression integration construct that is also a deletion construct. In other embodiments, deletion or disruption may be accomplished using a deletion construct that does not contain an exogenous gene or by other methods known in the art.

In various examples, the genetically modified yeast cells provided herein comprise a deletion or disruption of one or more native genes encoding an enzyme involved in ethanol fermentation, including for example pyruvate decarboxylase (PDC, converts pyruvate to acetaldehyde) and/or alcohol dehydrogenase (ADH, converts acetaldehyde to ethanol) genes. These modifications decrease the ability of the yeast cell to produce ethanol, thereby maximizing malonic acid, malonate, esters of malonic acid, or mixtures thereof production. However, in various examples the genetically modified yeast cells provided herein may be engineered to co-produce malonic acid, malonate, esters of malonic acid, or mixtures thereof and ethanol. In those embodiments, native genes encoding an enzyme involved in ethanol fermentation are not deleted or disrupted, and in various examples the yeast cells may comprise one or more exogenous genes that increase ethanol production.

In various examples, the genetically modified yeast cells provided herein comprise a deletion or disruption of one or more native genes encoding an enzyme that catalyzes a reverse reaction in a malonic acid, malonate, and esters of malonic acid fermentation pathway, including for example PEP carboxykinase (PCK), enzymes with OAA decarboxylase activity, or CYB2A or CYB2B (catalyzes the conversion of lactate to pyruvate). PCK catalyzes the conversion of PEP to OAA and vice versa, but exhibits a preference for the OAA to PEP reaction. To reduce the conversion of OAA to PEP, one or more copies of a native PCK gene may be deleted or disrupted. In various examples, yeast cells in which one or more native PCK genes have been deleted or disrupted may express one or more exogenous PCK genes that have been mutated to encode a polypeptide that favors the conversion of PEP to OAA. OAA decarboxylase catalyzes the conversion of OAA to pyruvate. Enzymes with OAA decarboxylase activity have been identified, such as malic enzyme (MAE) in yeast and fungi. To reduce OAA decarboxylase activity, one or more copies of a native gene encoding an enzyme with OAA decarboxylase activity may be deleted or disrupted. In various examples, yeast cells in which one or more native OAA decarboxylation genes have been deleted or disrupted may express one or more exogenous OAA decarboxylation genes that have been mutated to encode a polypeptide that catalyzes the conversion of pyruvate to OAA.

In some specific examples, select genes or combinations of genes can be overexpressed such that the production of malonic acid or malonate, can be enhanced. For example a copy number of the genes can be 2X, 3X, 4X, 5X, 6X, 7X, 8X, 9X, 10X, or higher. In some particular examples, it was found that by over expressing the following genes, combination of genes, or sub-combinations of genes: PYC, AAT, ADC, BAAT, or MSADh enhanced the production of malonic acid or malonate. Additionally, in some specific examples, select genes or combinations of genes can be deleted such that the production of malonic acid or malonate, can be enhanced. Examples of such genes, combinations of genes, or sub-combinations of genes include: 3-HP dehydrogenase, PDC, GPD1, or DLD (corresponding to SEQ ID No: 50). Additionally, in some specific examples, select genes or combinations of genes can be deleted such that the production of malonic acid or malonate, can be enhanced. Additionally, in some specific examples, select genes or combinations of genes can be deleted as neutral insertion sites such that the production of malonic acid or malonate, can be enhanced. Examples of such genes, combinations of genes, or sub-combination of genes include: a malate dehydrogenase (MDhb), an alcohol dehydrogenase (ADH) (e.g., ADH 9090 or ADH1202), Cyb2A, and Cyb2B.

In various examples, the genetically modified yeast cells provided herein comprise a deletion or disruption of one or more native genes encoding an enzyme involved in an undesirable reaction with a malonic acid, malonate, and esters of malonic acid fermentation pathway product or intermediate.

In various examples, the genetically modified yeast cells provided herein comprise a deletion or disruption of one or more native genes encoding an enzyme that has a neutral effect on a malonic acid, malonate, and esters of malonic acid fermentation pathway. Deletion or disruption of neutral genes allows for insertion of one or more exogenous genes without affecting native fermentation pathways.

In various examples, the yeast cells provided herein are malonic acid, malonate, esters of malonic acid, or mixtures thereof resistant yeast cells. A “malonic acid, malonate, esters of malonic acid, or mixtures thereof-resistant yeast cell” as used herein refers to a yeast cell that exhibits an average glycolytic rate of at least 2.5 g/L/hr in media containing 20 g/L or greater malonic acid, malonate, esters of malonic acid, or mixtures thereof at a pH of less than 6.0, less than about 5.0, less than about 4.0, or less than about 3.0. Such rates and conditions represent an economic process for producing malonic acid, malonate, esters of malonic acid, or mixtures thereof. In certain of these embodiments, the yeast cells may exhibit malonic acid, malonate, esters of malonic acid, or mixtures thereof resistance in their native form. In other embodiments, the cells may have undergone mutation and/or selection (e.g., chemostat selection or repeated serial subculturing) before, during, or after introduction of genetic modifications related to an active malonic acid, malonate, and esters of malonic acid fermentation pathway, such that the mutated and/or selected cells possess a higher degree of resistance to malonic acid, malonate, esters of malonic acid, or mixtures thereof than wild-type cells of the same species. For example, in some embodiments, the cells have undergone mutation and/or selection in the presence of malonic acid, malonate, esters of malonic acid, or mixtures thereof or lactic acid before being genetically modified with one or more exogenous malonic acid, malonate, and esters of malonic acid pathway genes. In various examples, mutation and/or selection may be carried out on cells that exhibit malonic acid, malonate, esters of malonic acid, or mixtures thereof resistance in their native form. Cells that have undergone mutation and/or selection may be tested for sugar consumption and other characteristics in the presence of varying levels of malonic acid, malonate, esters of malonic acid, or mixtures thereof in order to determine their potential as industrial hosts for malonic acid, malonate, esters of malonic acid, or mixtures thereof production. In addition to malonic acid, malonate, esters of malonic acid, or mixtures thereof resistance, the yeast cells provided herein may have undergone mutation and/or selection for resistance to one or more additional organic acids (e.g., lactic acid) or to other fermentation products, byproducts, or media components.

Selection, such as selection for resistance to malonic acid, malonate, esters of malonic acid, or mixtures thereof or to other compounds, may be accomplished using methods well known in the art. For example, as mentioned herein, selection may be chemostat selection. Chemostat selection uses a chemostat that allows for a continuous culture of microorganisms (e.g., yeast) wherein the specific growth rate and cell number can be controlled independently. A continuous culture is essentially a flow system of constant volume to which medium is added continuously and from which continuous removal of any overflow can occur. Once such a system is in equilibrium, cell number and nutrient status remain constant, and the system is in a steady state. A chemostat allows control of both the population density and the specific growth rate of a culture through dilution rate and alteration of the concentration of a limiting nutrient, such as a carbon or nitrogen source. By altering the conditions as a culture is grown (e.g., decreasing the concentration of a secondary carbon source necessary to the growth of the inoculum strain, among others), microorganisms in the population that are capable of growing faster at the altered conditions will be selected and will outgrow microorganisms that do not function as well under the new conditions. Typically such selection requires the progressive increase or decrease of at least one culture component over the course of growth of the chemostat culture. The operation of chemostats and their use in the directed evolution of microorganisms is well known in the art (see, e.g., Novick Proc Natl Acad Sci USA 36:708-719 (1950), Harder J Appl Bacteriol 43:1-24 (1977). Other methods for selection include, but are not limited to, repeated serial subculturing under the selective conditions as described in e.g., U.S. Pat. No. 7,629,162. Such methods can be used in place of, or in addition to, using the glucose limited chemostat method described above.

Yeast strains exhibiting the best combinations of growth and glucose consumption in malonic acid, malonate, esters of malonic acid, or mixtures thereof media as disclosed in the examples below are suitable host cells for various genetic modifications relating to malonic acid, malonate, and esters of malonic acid fermentation pathways. Yeast genera that possess the potential for a relatively high degree of malonic acid, malonate, and esters of malonic acid resistance, as indicated by growth in the presence of 75 g/L malonic acid, malonate, esters of malonic acid, or mixtures thereof or higher at a pH of less than 4, include for example Saccharomycescerevisiae, Candida,Kluyveromyces, Issatchenkia,Saccharomyces, Pichia,Schizosaccharomyces, Torulaspora, and Zygosaccharomyces. Species exhibiting malonic acid, malonate, esters of malonic acid, or mixtures thereof resistance include Saccharomycescerevisiae, Kluyveromyceslactis, Kluyveromyces marxianus, Yarrowialipolytica, Pichiakudriavzevii, Schizosaccharomycespombe.

Other wild-type yeast or fungi may be tested in a similar manner and identified to have acceptable levels of growth and glucose utilization in the presence of high levels of malonic acid, malonate, esters of malonic acid, or mixtures thereof as described herein. For example, Gross and Robbins (Hydrobiologia 433(103):91-109) have compiled a list of 81 fungal species identified in low pH (<4) environments that could be relevant to test as potential production hosts.

In various examples, the modified yeast cells provided herein are generated by incorporating one or more genetic modifications into a Crabtree-negative host yeast cell. In certain of these embodiments the host yeast cell belongs to the genus Issatchenkia, Candida, Pichia, or Kluyveromyces, and in certain of these embodiments the host cell belongs to the I. orientalis/P.fermentans clade. In certain of embodiments, the host cell is Saccharomycescerevisiae or C.lambica, or S.bulderi.

The I. orientalis/P.fermentans clade is the most terminal clade that contains at least the species I.orientalis, Pichiagaleiformis, Pichia sp. YB-4149 (NRRL designation), Candidaethanolica, Pichiadeserticola, Pichiamembranifaciens, and P.fermentans. Members of the I. orientalis/P.fermentans clade are identified by analysis of the variable D1/D2 domain of the 26S ribosomal DNA of yeast species, using the method described by Kurtzman and Robnett in “Identification and Phylogeny of Ascomycetous Yeasts from Analysis of Nuclear Large Subunit (26S) Ribosomal DNA Partial Sequences,” Antonie van Leeuwenhoek 73:331-371, 1998, incorporated herein by reference (see especially p. 349). Analysis of the variable D1/D2 domain of the 26S ribosomal DNA from hundreds of ascomycetes has revealed that the I. orientalis/P.fermentans clade contains very closely related species. Members of the I. orientalis/P.fermentans clade exhibit greater similarity in the variable D1/D2 domain of the 26S ribosomal DNA to other members of the clade than to yeast species outside of the clade. Therefore, other members of the I. orientalis/P.fermentans clade can be identified by comparison of the D1/D2 domains of their respective ribosomal DNA and comparing to that of other members of the clade and closely related species outside of the clade, using Kurtzman and Robnett’s methods.

A suitable host cell may possess one or more favorable characteristics in addition to malonic acid, malonate, esters of malonic acid, or mixtures thereof resistance and/or low pH growth capability. For example, potential host cells exhibiting malonic acid, malonate, esters of malonic acid, or mixtures thereof resistance may be further selected based on glycolytic rates, specific growth rates, thermotolerance, tolerance to biomass hydrolysate inhibitors, overall process robustness, and so on. These criteria may be evaluated prior to any genetic modification relating to a malonic acid, malonate, and esters of malonic acid fermentation pathway, or they may be evaluated after one or more such modifications have taken place.

Because most yeast are native producers of ethanol, elimination or severe reduction in the enzyme catalyzing the first step in ethanol production from pyruvate (PDC) is required for sufficient yield of an alternate product. In Crabtree-positive yeast such as Saccharomyces, a deleted or disrupted PDC gene causes the host to acquire an auxotrophy for two-carbon compounds such as ethanol or acetate, and causes a lack of growth in media containing glucose. Mutants capable of overcoming these limitations can be obtained using progressive selection for acetate independence and glucose tolerance (see, e.g., van Maris Appl Environ Microbiol 70:159 (2004)). Therefore, in various examples a suitable yeast host cell is a Crabtree-negative yeast cell, in which PDC deletion strains are able to grow on glucose and retain C2 prototrophy.

The level of gene expression and/or the number of exogenous genes to be utilized in a given cell will vary depending on the yeast species selected. For fully genome-sequenced yeasts, whole-genome stoichiometric models may be used to determine which enzymes should be expressed to develop a desired pathway malonic acid, malonate, and esters of malonic acid fermentation pathway. Whole-genome stoichiometric models are described in, for example, Hjersted et al., “Genome-scale analysis of Saccharomycescerevisiae metabolism and ethanol production in fed-batch culture,” Biotechnol. Bioeng. 2007; and Famili et al., “ Saccharomycescerevisiae phenotypes can be predicted by using constraint-based analysis of a genome-scale reconstructed metabolic network,” Proc. Natl. Acad. Sci. 2003, 100(23): 13134-9.

For yeasts without a known genome sequence, sequences for genes of interest (either as overexpression candidates or as insertion sites) can be obtained. Routine experimental design can be employed to test expression of various genes and activity of various enzymes, including genes and enzymes that function in a malonic acid, malonate, and esters of malonic acid pathway. Experiments may be conducted wherein each enzyme is expressed in the yeast individually and in blocks of enzymes up to and including all pathway enzymes, to establish which are needed (or desired) for improved malonic acid, malonate, and esters of malonic acid production. One illustrative experimental design tests expression of each individual enzyme as well as of each unique pair of enzymes, and further can test expression of all required enzymes, or each unique combination of enzymes. A number of approaches can be taken, as will be appreciated.

In various examples, fermentation methods are provided for producing malonic acid, malonate, esters of malonic acid, or mixtures thereof from a genetically modified yeast cell as provided herein. In some embodiments the fermentation methods can include simultaneous saccharification and fermentation. In some embodiments the fermentation method can carried out in aerobic, microaerobic or anaerobic conditions. By “microaerobic” it is meant that some oxygen is fed to the fermentation, and the microorganisms take up the oxygen fast enough such that the dissolved oxygen concentration averages less than about 2% of the saturated oxygen concentration under atmospheric air for at least five hours of the fermentation. Also, the average oxygen transfer rate of a microaerobic fermentation can be in a range of from about 3 mmol L⁻¹ h⁻¹ to about 80 mmol L⁻¹ h⁻¹, about 10 mmol l⁻¹ h⁻¹ to about 60 mmol l⁻¹ h⁻¹, about 25 to about 45 mmol l⁻¹ h⁻¹, less than, equal to, or greater than about 3 mmol l⁻¹ h⁻¹, 5, 10, 15, 20, 25, 30, 35, 40, 45, 50, 55, 60, 65, 70, 75, or about 80 mmol l⁻¹ h⁻¹. According to various embodiments, the oxygen transfer rate in the method can be proportional to the rate of production of malonic acid, malonate, or esters of malonic acid.

In various examples, these methods comprise culturing a genetically modified yeast cell as provided herein in the presence of at least one carbon source, allowing the cell to produce malonic acid, malonate, esters of malonic acid, or mixtures thereof for a period of time, and then isolating malonic acid, malonate, esters of malonic acid, or mixtures thereof produced by the cell from culture. The carbon source may be any carbon source that can be fermented by the provided yeast. The carbon source may be a twelve carbon sugar such as sucrose, a hexose sugar such as glucose or fructose, glycan or other polymer of glucose, glucose oligomers such as maltose, maltotriose and isomaltotriose, panose, and fructose oligomers. If the cell is modified to impart an ability to ferment pentose sugars, the fermentation medium may include a pentose sugar such as xylose, xylan or other oligomer of xylose, and/or arabinose. Such pentose sugars are suitably hydrolysates of a hemicellulose-containing biomass. In the case of oligomeric sugars, it may be necessary to add enzymes to the fermentation broth in order to digest these to the corresponding monomeric sugar for fermentation by the cell. In various examples, more than one type of genetically modified yeast cell may be present in the culture. Likewise, in various examples one or more native yeast cells of the same or a different species than the genetically modified yeast cell may be present in the culture.

In various examples, culturing of the cells provided herein to produce malonic acid, malonate, esters of malonic acid, or mixtures thereof may be divided up into phases. For example, the cell culture process may be divided into a cultivation phase, a production phase, and a recovery phase. One of ordinary skill in the art will recognize that the conditions used for these phases may be varied based on factors such as the species of microorganism being used, the specific malonic acid, malonate, and esters of malonic acid fermentation pathway utilized by the microorganism, the desired yield, or other factors.

The medium will typically contain nutrients as required by the particular cell, including a source of nitrogen (such as amino acids, proteins, inorganic nitrogen sources such as ammonia or ammonium salts, and the like), and various vitamins, minerals and the like. In some embodiments, the cells of the invention can be cultured in a chemically defined medium. In one example, the medium contains around 5 g/L ammonium sulfate, around 3 g/L potassium dihydrogen phosphate, around 0.5 g/L magnesium sulfate, trace elements, vitamins and around 150 g/L glucose. The pH may be allowed to range freely during cultivation, or may be buffered if necessary to prevent the pH from falling below or rising above predetermined levels. In various examples, the fermentation medium is inoculated with sufficient yeast cells that are the subject of the evaluation to produce an OD₆₀₀ of about 1.0. Unless explicitly noted otherwise, OD₆₀₀ as used herein refers to an optical density measured at a wavelength of 600 nm with a 1 cm pathlength using a model DU600 spectrophotometer (Beckman Coulter). The cultivation temperature may range from around 30-40° C., and the cultivation time may be up to around 120 hours.

In one example, the concentration of cells in the fermentation medium is typically in the range of about 0.1 to 20, from 0.1 to 5, or from 1 to 3 g dry cells/liter of fermentation medium during the production phase. The fermentation may be conducted aerobically, microaerobically, or anaerobically, depending on pathway requirements. If desired, oxygen uptake rate (OUR) can be varied throughout fermentation as a process control (see, e.g., WO03/102200). In some embodiments, the modified yeast cells provided herein are cultivated under microaerobic conditions characterized by an oxygen uptake rate from 2 to 45 mmol/L/hr, e.g., 2 to 25, 2 to 20, 2 to 15, 2 to 10, 10 to 45, 15 to 40, 20 to 35, or 25 to 35 mmol/L/hr. In various examples, the modified yeast cells provided herein may perform especially well when cultivated under microaerobic conditions characterized by an oxygen uptake rate of from 2 to 25 mmol/L/hr. The medium may be buffered during the production phase such that the pH is maintained in a range of about 3.0 to about 7.0, or from about 4.0 to about 6.0. Suitable buffering agents are basic materials that neutralize the acid as it is formed, and include, for example, calcium hydroxide, calcium carbonate, sodium hydroxide, potassium hydroxide, potassium carbonate, sodium carbonate, ammonium carbonate, ammonia, ammonium hydroxide and the like. In general, those buffering agents that have been used in conventional fermentation processes are also suitable here.

In those embodiments where a buffered fermentation is utilized, acidic fermentation products may be neutralized to the corresponding salt as they are formed. In these embodiments, recovery of the acid involves regeneration of the free acid. This may be done by removing the cells and acidulating the fermentation broth with a strong acid such as sulfuric acid. This results in the formation of a salt by-product. For example, where a calcium salt is utilized as the neutralizing agent and sulfuric acid is utilized as the acidulating agent, gypsum is produced as a salt by-product. This by-product is separated from the broth, and the acid is recovered using techniques such as liquid-liquid extraction, distillation, absorption, and others (see, e.g., T. B. Vickroy, Vol. 3, Chapter 38 of Comprehensive Biotechnology, (ed. M. Moo-Young), Pergamon, Oxford, 1985; R. Datta, et al., FEMS Microbiol Rev, 1995, 16:221-231; U.S. Pat. Nos. 4,275,234, 4,771,001, 5,132,456, 5,420,304, 5,510,526, 5,641,406, and 5,831,122, and WO93/00440.

In other embodiments, the pH of the fermentation medium may be permitted to drop during cultivation from a starting pH that is at or above the pKa of malonic acid, malonate, esters of malonic acid, or mixtures thereof, typically 4.5 or higher, to at or below the pKa of the acid fermentation product, e.g., less than 4.5 or 4.0, such as in the range of about 1.5 to about 4.5, in the range of from about 2.0 to about 4.0, or in the range from about 2.0 to about 3.5.

In still other embodiments, fermentation may be carried out to produce a product acid by adjusting the pH of the fermentation broth to at or below the pKa of the product acid prior to or at the start of the fermentation process. The pH may thereafter be maintained at or below the pKa of the product acid throughout the cultivation. In various examples, the pH may be maintained at less than 4.5 or 4.0, such as in a range of about 1.5 to about 4.5, in a range of about 2.0 to about 4.0, or in a range of about 2.0 to about 3.5.

In various examples of the methods provided herein, the genetically modified yeast cells produce relatively low levels of ethanol. In various examples, ethanol may be produced in a yield of 10% or less, in a yield of 2% or less, or even 0% ethanol. In certain of these embodiments, ethanol is not detectably produced. In other embodiments, however, malonic acid, malonate, esters of malonic acid, or mixtures thereof and ethanol may be coproduced. In these embodiments, ethanol may be produced at a yield of greater than 10%, greater than 25%, or greater than 50%.

In various examples of the methods provided herein, the final yield of malonic acid, malonate, esters of malonic acid, or mixtures thereof on the carbon source is at least 10%, at least 20%, at least 30%, at least 40%, at least 50%, or greater than 50% of the theoretical yield. The concentration, or titer, of malonic acid, malonate, esters of malonic acid, or mixtures thereof will be a function of the yield as well as the starting concentration of the carbon source. In various examples, the titer may reach at least 1-3, at least 5, at least 10, at least 20, at least 30, at least 40, at least 50, at least 100 g/L, at least 110 g/L, at least 120 g/L, at least 130 g/L, at least 140 g/L, at least 150 g/L, at least 160 g/L, at least 170 g/L, at least 180 g/L, at least 190 g/L, at least 200 g/L, at least 210 g/L, at least 220 g/L, at least 230 g/L, at least 240 g/L, at least 250 g/L, or in a range of from about 9 g/L to about 250 g/L, about 20 g/L to about 220 g/L, about 50 g/L to about 200 g/L, or about 100 g/L to about 150 g/L, at some point during the fermentation, and suitably at the end of the fermentation. In various examples, the final yield of malonic acid, malonate, esters of malonic acid, or mixtures thereof may be increased by altering the temperature of the fermentation medium, particularly during the production phase.

Once produced, any method known in the art can be used to isolate malonic acid, malonate, esters of malonic acid, or mixtures thereof from the fermentation medium. For example, common separation techniques can be used to remove the biomass from the broth, and common isolation procedures (e.g., extraction, distillation, and ion-exchange procedures) can be used to obtain the malonic acid, malonate, esters of malonic acid, or mixtures thereof from the microorganism-free broth. In addition, malonic acid, malonate, esters of malonic acid, or mixtures thereof can be isolated while it is being produced, or it can be isolated from the broth after the product production phase has been terminated.

Malonic acid, malonate, esters of malonic acid, or mixtures thereof produced using the methods disclosed herein can be chemically converted into other organic compounds. For example, malonic acid, malonate, esters of malonic acid, or mixtures thereof can be hydrogenated to form 1,3 propanediol, a valuable polyester monomer. Propanediol also can be created from malonic acid, malonate, esters of malonic acid, or mixtures thereof using polypeptides having oxidoreductase activity in vitro or in vivo. Hydrogenating an organic acid such as malonic acid, malonate, esters of malonic acid, or mixtures thereof can be performed using any method such as those used to hydrogenate succinic acid and/or lactic acid. For example, malonic acid, malonate, esters of malonic acid, or mixtures thereof can be hydrogenated using a metal catalyst. In another example, malonic acid, malonate, esters of malonic acid, or mixtures thereof can be dehydrated to form acrylic acid using any known method for performing dehydration reactions. For example, malonic acid, malonate, esters of malonic acid, or mixtures thereof can be heated in the presence of a catalyst (e.g., a metal or mineral acid catalyst) to form acrylic acid.

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention. It will be understood that many variations can be made in the procedures herein described while still remaining within the bounds of the present invention. It is the intention of the inventors that such variations are included within the scope of the invention.

### EXAMPLES

Various embodiments of the present disclosure can be better understood by reference to the following Examples which are offered by way of illustration. The present disclosure is not limited to the Examples given herein.

### Example 1: Creation of Strains of Saccharomyces Cerevisiae Used in the Testing of Enzymes

### Strain 1.1

Strain 1.1 is a Saccharomycescerevisiae CEN.PK 113-7D haploid strain in which the URA3 open reading frame has been deleted from the genome using methods known in the art, making the strain unable to grown on media that does not contain uracil.

### Strain 1.2

Strain 1.1 is transformed with SEQ ID NO: 1 and SEQ ID NO 2. SEQ ID NO: 1 contains: i) 5’ homology to the integration locus FCY1, ii) an expression cassette for a beta-alanine aminotransferase PYD4 from Pichiakudriavzevii, SEQ ID NO: 3, expressed by the TDH3 promoter, and iii) the 5’ half of a ScURA3 expression cassette flanked by a loxP recombination site. SEQ ID NO: 2 contains: i) 3’ homology to the integration locus FCY1, ii) an expression cassette for a beta-alanine aminotransferase PYD4 from Pichiakudriavzevii, SEQ ID NO: 3, expressed by the TDH3 promoter, and iii) the 3’ half of a ScURA3 expression cassette flanked by a loxP recombination site. Transformants are selected on synthetic complete media lacking uracil. (ScD-Ura). Resulting transformants are streaked for single colony isolation on ScD-Ura. A single colony is selected. Correct integration of SEQ ID NO: 1 and SEQ ID NO: 2 into the FCY1 integration locus is verified by PCR in the single colony. A PCR verified isolate is designated Strain 1.2.

### Strain 1.3

Strain 1.2 is transformed with SEQ ID NO: 4. SEQ ID NO: 4 contains the following elements: i) an expression cassette for an aminoglycoside O-phosphotransferase gene; ii) an expression cassette for a cre recombinase from P1 bacteriophage; iii) an expression cassette containing the native URA3, and iv) the Saccharomyces cerevisiae CEN6 centromere. Transformants are selected on YPD media containing 200 mg/L G418 sulfate. Resulting transformants are streaked for single colony isolation on YPD media containing 200 mg/L G418 sulfate. A single colony is selected. The colony is grown on YPD media to allow for loss of the plasmid. Loss of the ScURA3 expression cassette is verified by PCR. The PCRverified isolate is designated Strain 1.3.

### Strain 1.4

Strain 1.3 is transformed with SEQ ID NO: 5. SEQ ID NO: 5 contains i) 5’ homology to the integration locus YMR226c, ii) an expression cassette for the Aspergillusnidulans acetamidase gene, and iii) 3’ homology to the integration locus YMR226c. Transformants are selected on synthetic complete media lacking uracil. (ScD-Ura). Resulting transformants are streaked for single colony isolation on ScD-Ura. A single colony is selected. Correct integration of SEQ ID NO: 5 is verified by PCR in the single colony. A PCR verified isolate is designated Strain 1.4.

### Strain 1.5, Strains 1.7 thru 1.12, Strain 1.20

Strain 1.1 is transformed with the first Seq ID, SEQ ID NO:6, from the Construct Seq ID column of Table 3-2. The Seq ID’s listed in Table 3-2 contain i) an expression cassette for an aminoglycoside O-phosphotransferase gene; ii) an expression cassette for a malonate-semialdehyde dehydrogenase; iii) an expression cassette containing the native URA3, and iv) the Saccharomyces cerevisiae 2 micron origin of replication. Transformants are selected on synthetic complete media lacking uracil. (ScD-Ura). Resulting transformants are streaked for single colony isolation on ScD-Ura. A single colony is selected. A PCR verified isolate is designated Strain 1.5 as given in the Strain column of Table 3-2. This process is repeated for each of the Seq ID NO’s 10, 12, 14, 16, 18, 20, and 22 from the Construct Seq ID column of Table 3-2 resulting in Strains 1.7 thru strains 1.12 and Strain 1.20 designated in the Strain column of Table 3-2.

### Strain 1.6, Strains 1.13 thru 1.16

Strain 1.4 is transformed with the second Seq ID, SEQ ID NO: 8, from the Construct Seq ID column of Table 3-2. The Seq ID’s listed in Table 3-2 contain i) an expression cassette for an aminoglycoside O-phosphotransferase gene; ii) an expression cassette for a malonate-semialdehyde dehydrogenase; iii) an expression cassette containing the native URA3, and iv) the Saccharomyces cerevisiae 2 micron origin of replication. Transformants are selected on synthetic complete media lacking uracil. (ScD-Ura). Resulting transformants are streaked for single colony isolation on ScD-Ura. A single colony is selected. A PCR verified isolate is designated Strain 1.6 as given in the Strain column of Table 3-2. This process is repeated for each of the Seq ID NO’s 24, 26, 28, and 30 from the Construct Seq ID column of Table 3-2 resulting in Strains 1.13 thru 1.16 designated in the Strain column of Table 3-2.

### Strain 1.17

Strain 1.4 is transformed with SEQ ID NO: 6. SEQ ID NO: 6 contains i) an expression cassette for an aminoglycoside O-phosphotransferase gene; ii) an expression cassette encoding for a polypeptide from E.coli, SEQ ID NO: 7; iii) an expression cassette containing the native URA3, and iv) the Saccharomyces cerevisiae 2 micron origin of replication. Transformants are selected on synthetic complete media lacking uracil. (ScD-Ura). Resulting transformants are streaked for single colony isolation on ScD-Ura. A single colony is selected. A PCR verified isolate is designated Strain 1.17.

### Strain 1.18

Strain 1.4 is transformed with SEQ ID NO: 8. SEQ ID NO: 8 contains i) an expression cassette for an aminoglycoside O-phosphotransferase gene; ii) an expression cassette encoding for a polypeptide from Acetobacterghanensis, SEQ ID NO: 9; iii) an expression cassette containing the native URA3, and iv) the Saccharomyces cerevisiae 2 micron origin of replication. Transformants are selected on synthetic complete media lacking uracil. (ScD-Ura). Resulting transformants are streaked for single colony isolation on ScD-Ura. A single colony is selected. A PCR verified isolate is designated Strain 1.18.

### Strain 1.19

Strain 1.4 is transformed with SEQ ID NO: 10. SEQ ID NO: 10 contains i) an expression cassette for an aminoglycoside O-phosphotransferase gene; ii) an expression cassette encoding for a polypeptide from Paraburkholderia xenovorans, SEQ ID NO: 11; iii) an expression cassette containing the native URA3, and iv) the Saccharomyces cerevisiae 2 micron origin of replication. Transformants are selected on synthetic complete media lacking uracil. (ScD-Ura). Resulting transformants are streaked for single colony isolation on ScD-Ura. A single colony is selected. A PCR verified isolate is designated Strain 1.19.

### Example 2: Construction of Strains of Pichia Kudriavzevii for the Testing Of Enzymes

### Strain 2.1

Strain 2.1 is Strain C in WO2017024150, which is deleted for both alleles of the URA3 gene, making the strain unable to grown on media that does not contain uracil.

### Strain 2.2

Strain 2.1 is transformed with SEQ ID NO: 32. SEQ ID NO: 32 contains the following elements: i) 5’ homology to the integration locus PDC1, ii) an expression cassette containing the native URA3, and iii) 3’ homology to the integration locus PDC1. Transformants are selected on ScD -Ura media. Resulting transformants are streaked for single colony isolation on ScD -Ura media. A single colony is selected and correct integration of SEQ ID NO 32 into the PDC 1 locus is verified by PCR. The PCR verified isolate is designated Strain 2.2.

### Strain 2.3

Strain 2.2 is transformed with SEQ ID NO: 33. SEQ ID NO: 33 contains the following elements: i) 5’ homology to the integration locus PDC1, ii) an expression cassette containing the ScMEL5 expressed by the native PGK1 promoter, and iii) 3’ homology to the integration locus PDC1. Transformants are selected on YNB + 20 g/L Melibiose + X-α-gal media. Resulting transformants are streaked for single colony isolation on YNB + 20 g/L Melibiose + X-α-gal media. A single colony is selected and correct integration of SEQ ID NO: 32 and SEQ ID NO: 33 into the PDC1 locus is verified by PCR. The PCR verified isolate is designated Strain 2.3.

### Strain 2.4

Strain 2.3 is transformed with SEQ ID NO: 34. SEQ ID NO: 34 contains the following elements: i) a Cre recombinase expressed by the native PDC1 promoter, ii) an expression cassette containing the ScSUC2 expressed by the native PGK1 promoter, and iii) an autonomously replicating sequence (ARS). Transformants are selected on YNB + 20 g/L Sucrose + X-α-gal media. Resulting transformants are streaked for single colony isolation on YPD + X-α-gal media. A single white colony is selected and correct recycling of markers in SEQ ID NO: 32 & 34 at the PDC1 locus is verified by PCR. The PCR verified isolate is designated Strain 2.4.

### Strain 2.5

Strain 2.4 is transformed with SEQ ID NO: 35 and SEQ ID NO: 37. SEQ ID NO: 35 contains: i) 5’ homology to the integration locus MDHB, ii) an expression cassette for an aspartate decarboxylase ADC from Danaus plexippus, SEQ ID NO: 36, expressed by the PDC1 promoter, and iii) the 5’ half of a IoURA3 expression cassette. SEQ ID NO 37 contains: i) 3’ half of a IoURA3 expression cassette flanked by a IoURA3 promoter fragment for recombination ii) an expression cassette for an aspartate decarboxylase ADC from Danaus plexippus, SEQ ID NO: 36, expressed by the TDH3 promoter, and iii) 3’ homology to the integration locus MDHB,. Transformants are selected on synthetic complete media lacking uracil. (ScD-Ura). Resulting transformants are streaked for single colony isolation on ScD-Ura. A single colony is selected. Correct integration of SEQ ID NO: 35 and SEQ ID NO: 37 into the MDHB integration locus is verified by PCR in the single colony. A PCR verified isolate is designated Strain 2.5.

### Strain 2.6

Strain 2.5 is transformed with SEQ ID NO: 38 and SEQ ID NO: 39. SEQ ID NO: 38 contains: i) 5’ homology to the integration locus MDHB, ii) an expression cassette for an aspartate decarboxylase ADC from Danaus plexippus, SEQ ID NO: 36, expressed by the PDC1 promoter, and iii) the 5’ half of a hygromycin resistance HPH expression cassette flanked by loxP recombination site. SEQ ID NO: 39 contains: i) 3’ half of a hygromycin resistance HPH expression cassette flanked by a loxP recombination site ii) an expression cassette for an aspartate decarboxylase ADC from Danaus plexippus, SEQ ID NO: 36, expressed by the TDH3 promoter, and iii) 3’ homology to the integration locus MDHB,. Transformants are selected on YPD media containing hygromycin. (YPD + Hygro300). Resulting transformants are streaked for single colony isolation on YPD + Hygro300 and a single colony is selected. Correct integration of SEQ ID NO: 38 and SEQ ID NO: 39 into the MDHB integration locus is verified by PCR in the single colony. A PCR verified isolate is designated Strain 2.6.

### Strain 2.7

Strain 2.6 is transformed with SEQ ID NO: 40. SEQ ID NO: 40 contains the following elements: i) 5’ homology to the integration locus YMR226c, ii) an expression cassette containing the ScMEL5 expressed by the native PGK1 promoter, and iii) 3’ homology to the integration locus YMR226c. Transformants are selected on YNB + Melibiose + X-α-gal solid media. Resulting transformants are streaked for single colony isolation on YNB + Melibiose + X-α-gal media. A single colony is selected and correct integration of SEQ ID NO: 40 into the YMR226c locus is verified by PCR. The PCR verified isolate is designated Strain 2.7.

### Strain 2.8

Strain 2.7 is grown overnight in YPD media. The resulting culture is plated onto ScD + 5-fluoroorotic acid (FOA) agar plates for selection of IoURA3 marker loop outs. Resulting colonies are picked and struck for isolation on Sc-FOA solid media, single colonies are then PCR verified for IoURA3 loop out. The PCR verified isolate is designated Strain 2.8.

TABLE 2-2

ScD + 5-fluoroorotic acid (FOA) agar plates

| | |
|---|---|
| Bacto™ Agar | 20.0 g |
| Yeast Nitrogen Base W/O AA | 6.7 g |
| Sc-Ura AA Dropout Mix | 1.9 g |
| Anhydrous Glucose | 20.0 g |
| Uracil | 1.0 g |
| Uradine | 1.0 g |
| 5-Fluoroorotic Acid (FOA) (Zymo Research #F9003) | 15 ml |
| Distilled Water | 1L |

### Strain 2.9

Strain 2.8 is transformed with SEQ ID NO: 41 and SEQ ID NO: 42. SEQ ID NO: 41 contains: i) 5’ homology to the integration locus YMR226c, and ii) the 5’ half of a IoURA3 expression cassette flanked by loxP sites for recombination. SEQ ID NO: 42 contains: i) the 3’ half of a IoURA3 expression cassette flanked by loxP sites for recombination ii) an expression cassette for a malonate-semialdehyde dehydrogenase, SEQ ID NO: 27, expressed by the TDH3 promoter, iii) an expression cassette for a malonate-semialdehyde dehydrogenase, SEQ ID NO: 27, expressed by the TEF2 promoter, and iv) 3’ homology to the integration locus YMR226c. Transformants are selected on synthetic complete media lacking uracil. (ScD-Ura). Resulting transformants are streaked for single colony isolation on ScD-URA and a single colony is selected. Correct integration of SEQ ID NO: 41 and SEQ ID NO: 42 into the YMR226c integration locus is verified by PCR in the single colony. A PCR verified isolate is designated Strain 2.9.

### Strain 2.10

Strain 2.8 is transformed with SEQ ID NO: 43 and SEQ ID NO: 42. SEQ ID NO 43 contains: i) 5’ homology to the integration locus YMR226c, ii) an expression cassette for a malonate-semialdehyde dehydrogenase, SEQ ID NO: 27, expressed by the PENO1 promoter, iii) an expression cassette for a malonate-semialdehyde dehydrogenase, SEQ ID NO: 27, expressed by the PDC1 promoter and iv) the 5’ half of a IoURA3 expression cassette flanked by loxP sites for recombination. SEQ ID NO: 42 contains: i) the 3’ half of a IoURA3 expression cassette flanked by loxP sites for recombination ii) an expression cassette for a malonate-semialdehyde dehydrogenase, SEQ ID NO: 27, expressed by the TDH3 promoter, iii) an expression cassette for a malonate-semialdehyde dehydrogenase, SEQ ID NO: 27, expressed by the TEF2 promoter, and iv) 3’ homology to the integration locus YMR226c. Transformants are selected on synthetic complete media lacking uracil. (ScD-Ura). Resulting transformants are streaked for single colony isolation on ScD-URA and a single colony is selected. Correct integration of SEQ ID NO: 43 and SEQ ID NO: 42 into the YMR226c integration locus is verified by PCR in the single colony. A PCR verified isolate is designated Strain 2.10.

### Strain 2.11

Strain 2.8 is transformed with SEQ ID NO: 41 and SEQ ID NO: 44. SEQ ID NO: 41 contains: i) 5’ homology to the integration locus YMR226c, and ii) the 5’ half of a IoURA3 expression cassette flanked by loxP sites for recombination. SEQ ID NO: 44 contains: i) the 3’ half of a IoURA3 expression cassette flanked by loxP sites for recombination ii) an expression cassette for a malonate-semialdehyde dehydrogenase, SEQ ID NO: 45, expressed by the TDH3 promoter, iii) an expression cassette for a malonate-semialdehyde dehydrogenase, SEQ ID NO: 45, expressed by the TEF2 promoter, and iv) 3’ homology to the integration locus YMR226c. Transformants are selected on synthetic complete media lacking uracil. (ScD-Ura). Resulting transformants are streaked for single colony isolation on ScD-URA and a single colony is selected. Correct integration of SEQ ID NO: 41 and SEQ ID NO: 44 into the YMR226c integration locus is verified by PCR in the single colony. A PCR verified isolate is designated Strain 2.11.

### Strain 2.12

Strain 2.8 is transformed with SEQ ID NO: 41 and SEQ ID NO: 46. SEQ ID NO: 41 contains: i) 5’ homology to the integration locus YMR226c, and ii) the 5’ half of a IoURA3 expression cassette flanked by loxP sites for recombination. SEQ ID NO: 46 contains: i) the 3’ half of a IoURA3 expression cassette flanked by loxP sites for recombination ii) an expression cassette for a malonate-semialdehyde dehydrogenase, SEQ ID NO: 29, expressed by the TDH3 promoter, iii) an expression cassette for a malonate-semialdehyde dehydrogenase, SEQ ID NO: 29, expressed by the TEF2 promoter, and iv) 3’ homology to the integration locus YMR226c. Transformants are selected on synthetic complete media lacking uracil. (ScD-Ura). Resulting transformants are streaked for single colony isolation on ScD-URA and a single colony is selected. Correct integration of SEQ ID NO: 41 and SEQ ID NO: 46 into the YMR226c integration locus is verified by PCR in the single colony. A PCR verified isolate is designated Strain 2.12.

### Strain 2.13

Strain 2.8 is transformed with SEQ ID NO: 41 and SEQ ID NO: 47. SEQ ID NO: 41 contains: i) 5’ homology to the integration locus YMR226c, and ii) the 5’ half of a IoURA3 expression cassette flanked by loxP sites for recombination. SEQ ID NO: 47 contains: i) the 3’ half of a IoURA3 expression cassette flanked by loxP sites for recombination ii) an expression cassette for a malonate-semialdehyde dehydrogenase, SEQ ID NO: 11, expressed by the TDH3 promoter, iii) an expression cassette for a malonate-semialdehyde dehydrogenase, SEQ ID NO: 11, expressed by the TEF2 promoter, and iv) 3’ homology to the integration locus YMR226c. Transformants are selected on synthetic complete media lacking uracil. (ScD-Ura). Resulting transformants are streaked for single colony isolation on ScD-URA and a single colony is selected. Correct integration of SEQ ID NO: 41 and SEQ ID NO: 47 into the YMR226c integration locus is verified by PCR in the single colony. A PCR verified isolate is designated Strain 2.13.

### Example 3: Screening of Enzymes with the Malonate-Semialdehyde Dehydrogenase Assay for Enzyme Produced by Saccharomyces Cerevisiae

### Production and Lysis of Saccharomyces Cerevisiae Enzyme

The capability of the enzyme to convert malonate-semialdehyde (MSA) to malonic acid is evaluated by the following protocol.

The malonate-semialdehyde dehydrogenase (MSADh) candidate gene is synthesized and cloned into the yeast expression vector. The resulting MSADh expression vector is transformed into Saccharomycescerevisiae Strain by methods as described in the state of the art. The strain is taken from an ScD-Ura agar plate and used to inoculate 3 mL of fresh SCD-Ura media (SC-Ura, 100 g/L glucose, 0.1 M MES). The culture is incubated at 30° C., 250 rpm, 70% humidity overnight (16 hours). The overnight culture is used to inoculate 25 mL of fresh SCD-Ura media to an OD₆₀₀ of 0.2 and incubated at 30° C., 250 rpm, 70% humidity for 8 hours. 10 OD equivalent of the cells are harvested by centrifugation at 4000 rpm for 10 minutes at 4° C. The pellets are washed with 0.5 mL cold water, centrifuged at 4000 rpm for 10 minutes at 4° C., and stored at -80° C.

Pellets are thawed and lysed with 0.25 mL lysis solution (Yeastbuster, EMDmillipore) with 1X THP ( EMDmillipore), 1X HALT protease inhibitor ( ThermoScientific), and 0.5 ul benzonase) for 20 minutes at room temperature with gentle rocking. Cell debris is removed by centrifugation and the supernatant is desalted using a Zeba spin column ( ThermoScientific) equilibrated with 1X PBS. Protein concentration is determined using Pierce660 protein assay ( ThermoScientific) and normalized to 3 mg/mL.

TABLE 3-1

SCD-Ura Plates

| | |
|---|---|
| Difco™ Yeast Nitrogen Base without amino acids (BD #291940) | 6.7 g |
| Glucose | 20 g |
| Agar | 20 g |
| SC-Ura Mixture (MP Biomedicals #4410-622) | 2 g |
| Distilled H₂O | to 1 L |

Autoclave at 110° C. for 25 min

### Malonate-Semialdehyde Dehydrogenase Assay for Enzyme Produced by Saccharomyces Cerevisiae

The activity of an MSADh is assayed by monitoring concentration of NADH spectrophotometrically at 340 nm in 50 mM HEPES pH8, 1 mM DTT, 1 mM NAD+, and 3 mM malonate-semialdehyde. Vmax corresponding to the steepest slope is determined by SoftMax Pro 7 (version 7.0.2) software and converted to activity (nmol min⁻¹ mg⁻¹) using methods as known in the art. SEQ ID 25 has an additional alanine to valine substitution; this substitution has been shown to have minimal effect on activity.

The strains in Table 3-2 are screened using the malonate-semialdehyde dehydrogenase assay for enzyme produced by Saccharomyces cerevisiae.

TABLE 3-2

| Construct SEQ ID | MSADh Amino Acid SEQ ID | Strain | MSADh activity (nmol min⁻¹ mg⁻¹) |
|---|---|---|---|
| 6 | 7 | Strain 1.5 | 24.6 |
| 8 | 9 | Strain 1.6 | 27.9 |
| 10 | 11 | Strain 1.7 | 22.4 |
| 12 | 13 | Strain 1.8 | 4.2 |
| 14 | 15 | Strain 1.20 | 8.9 |
| 16 | 17 | Strain 1.9 | 7.2 |
| 18 | 19 | Strain 1.10 | 8.2 |
| 20 | 21 | Strain 1.11 | 9.5 |
| 22 | 23 | Strain 1.12 | 49.3 |
| 24 | 25 | Strain 1.13 | 166.9 |
| 26 | 27 | Strain 1.14 | 167.7 |
| 28 | 29 | Strain 1.15 | 71.7 |
| 30 | 31 | Strain 1.16 | 73.1 |

The results from the malonate-semialdehyde dehydrogenase assay demonstrated that the enzymes of table 3-2 showed non-zero enzymatic activity and were, therefore, suitable candidates to include in the various host microorganisms described herein. From those results, all of the enzymes showed promise as leads for further modification. For example, the enzymes represented by SEQ IDs 7, 9, 11, and 23 alone showed some of the highest activity. In particular, those enzymes were modified with the substitution from a phenylalanine to a tryptophan at select amino acid residue locations as represented by SEQ IDs 25, 27, 29, and 31. The results obtained in conjunction with these substitutions showed that it was possible to increase the activity of select enzymes and suggests that changing phenylalanine to tryptophan at the corresponding position in these enzymes could increase the activity of the enzyme.

### Example 4: Assessing in Vivo Enzyme Activity by Measuring Conversion Of Beta-Alanine to Malonic Acid in Yeasts Containing Heterologous Enzymes

Strains 1.17 thru 1.19 are streaked out for single colonies on URA selection plates and incubated at room temperature for 2-3 days until single colonies are visible. A 10 microliter loop-full of cells from the selection plates is scraped into a 250 ml baffled Erlenmeyer shake flask containing 25 ml sterile seed medium and incubated at 34° C. at 250 RPM and 70% humidity in an Infors Multitron shaking incubator with a 2.5 cm throw (model AJ125C) for 16-20 hours. Optical density (OD600) is measured. Optical density is measured at wavelength 600 nm with a 1 cm pathlength using a model Genesys20 Spectrophotometer (Thermo Scientific, model 4001/4). Dry cell mass is calculated from the measured OD600 value using an experimentally derived conversion factor of 1.94 OD600 units per 1 g/L dry cell mass.

A second 250 ml baffled Erlenmeyer shake flask with 25 ml sterile seed medium is inoculated with cells from the first shake flask to reach an initial OD600 of 0.5. This shake flask is incubated under the same conditions as above for 4-8 hours.

A fresh 250 ml non-baffled shake flask with a vented screw cap with gas permeable membrane containing 25 ml sterile production media is inoculated with a volume of cells from the second seed flask to result in an initial OD600 of 0.2.

The seed medium is a sterilized aqueous solution of yeast nitrogen base without amino acids (BD #291940) (6.7 g/L), ScD amino acids without ura mixture (MP Biomedicals #4410-622) (2 g/L), and glucose (20 g/L). The shake flask production medium is a sterilized, 5.8 pH aqueous solution of urea (2.3 g/L), magnesium sulphate heptahydrate (0.5 g/L), potassium phosphate monobasic (3 g/L), trace element solution (1 ml/L), vitamin solution (1 ml/L), maltodextrin (100 g/L), and 2-(N-Morpholino) ethanesulfonic acid (MES) (39.05 g/L). Amyloglucosidase from Aspergillusniger (Sigma A7095) (50 ul/L) is added immediately prior to inoculation. For strains lacking the URA3 gene (URA-) 100 mg/L uracil is added to the media. The trace element solution is a sterilized, pH 4.0 aqueous solution of EDTA (15.0 g/L), zinc sulfate heptahydrate (4.5 g/L), manganese chloride dehydrate (1.2 g/L), cobalt(II) chloride hexahydrate (0.3 g/L), copper(II)sulfate pentahydrate (0.3 g/L), disodium molybdenum dehydrate (0.4 g/L), calcium chloride dehydrate (4.5 g/L), iron sulphate heptahydrate (3 g/L), boric acid (1.0 g/L), and potassium iodide (0.1 g/L). The vitamin solution is a sterilized, pH 6.5 aqueous solution of biotin (D-; 0.05 g/L), calcium pantothenate (D+; 1 g/L), nicotinic acid (5 g/L), myo-inositol (25 g/L), thiamine hydrochloride (1 g/L), pyridoxine hydrochloride (1 g/L), and p-aminobenzoic acid (0.2 g/L).

The inoculated flask is incubated at 34° C. at 250 RPM and 70% humidity in an Infors Multitron shaking incubator with a 2.5 cm throw (model AJ125C) for 72 hours. After 16 hours incubation, 10 g/L beta-alanine (Alfa Aesar A16665) is added to the shake flask. A 0.35 ml sample is taken immediately after inoculation. Samples of 0.7 ml are taken at 24, 48 hours after inoculation. At 72 hours, 1 ml sample is taken. Malonic acid concentration in the samples are determined by high performance liquid chromatography with refractive index detector for all time points, and additionally by high pressure ion chromatography for the sample taken at 72 hours.

TABLE 4-1

| Strain no | MSADh Amino Acid SEQ ID | Malonic Acid (PPM) | Standard Deviation (PPM) |
|---|---|---|---|
| Strain 1.17 | 7 | 0.8 | 0.53 |
| Strain 1.18 | 9 | 88.6 | 3.58 |
| Strain 1.19 | 11 | 17.9 | 1.74 |

The results displayed in Table 4-1 demonstrate 1) that Strains 1.17 thru 1.19 each comprising one of the enzymes generated are able to convert beta-alanine to malonic acid and 2) demonstrates the in vivo activity of SEQ ID NOs: 7, 9 and 11 in a yeast host. Moreover, the data shows that SEQ ID NOs: 9 and 11 are preferred over SEQ ID NO: 7 when the host organism is a yeast.

### Example 5: Malonic Acid Production From a Fermentation with a Controlled Release of Glucose Substrate From Maltodextrin

Strains 2.11 and 2.12 are streaked out for single colonies on X-gal selection plates and incubated at room temperature for 2-3 days until single colonies are visible. A 10 microliter loop-full of cells from the selection plates is scraped into a 250 ml baffled Erlenmeyer shake flask containing 40 ml sterile seed medium and incubated at 30° C. at 250 RPM and 70% humidity in an Infors Multitron shaking incubator with a 2.5 cm throw (model AJ125C) for 16-20 hours. Optical density (OD600) is measured. Optical density is measured at wavelength 600 nm with a 1 cm pathlength using a model Genesys20 Spectrophotmeter (Thermo Scientific, model 4001/4). Dry cell mass is calculated from the measured OD600 value using an experimentally derived conversion factor of 1.77 OD600 units per 1 g/L dry cell mass.

A new 250 ml baffled Erlenmeyer shake flask with 30 ml sterile seed medium is inoculated with cells from the first shake flask to reach an initial OD600 of 1.2. This shake flask is incubated under the same conditions as above for 4-8 hours.

A new 250 ml non-baffled shake flask with a vented screw cap with gas permeable membrane containing 30 ml sterile production media is inoculated with cells from the second seed flask to an initial OD600 of 0.1.

The seed medium is a sterilized, pH 6.2 aqueous solution of urea (2.3 g/L), magnesium sulphate heptahydrate (0.5 g/L), potassium phosphate monobasic (3 g/L), trace element solution (1 ml/L), vitamin solution (1 ml/L), glucose (25 g/L), ), and 2-(N-Morpholino) ethanesulfonic acid (MES) (13.7 g/L). The shake flask production medium is a sterilized, 6.2 pH aqueous solution of urea (2.3 g/L), magnesium sulphate heptahydrate (0.5 g/L), potassium phosphate monobasic (3 g/L), trace element solution (1 ml/L), vitamin solution (1 ml/L), maltodextrin (100 g/L), and 2-(N-Morpholino) ethanesulfonic acid (MES) (39.05 g/L). Amyloglucosidase from Aspergillusniger (Sigma A7095) (25 ul/L) is added immediately prior to inoculation. For strains lacking the URA3 gene (URA-) 100 mg/L uracil is added to the media. The trace element solution is a sterilized, pH 4.0 aqueous solution of EDTA (15.0 g/L), zinc sulfate heptahydrate (4.5 g/L), manganese chloride dehydrate (1.2 g/L), cobalt(II) chloride hexahydrate (0.3 g/L), copper(II)sulfate pentahydrate (0.3 g/L), disodium molybdenum dehydrate (0.4 g/L), calcium chloride dehydrate (4.5 g/L), iron sulphate heptahydrate (3 g/L), boric acid (1.0 g/L), and potassium iodide (0.1 g/L). The vitamin solution is a sterilized, pH 6.5 aqueous solution of biotin (D-; 0.05 g/L), calcium pantothenate (D+; 1 g/L), nicotinic acid (5 g/L), myo-inositol (25 g/L), pyridoxine hydrochloride (1 g/L), and p-aminobenzoic acid (0.2 g/L).

The inoculated flask is incubated at 30° C. at 325 RPM and 70% humidity in an Infors Multitron shaking incubator with a 2.5 cm throw (model AJ125C) for 72 hours. A sample of 0.35 ml is taken at 0 hours incubation. Samples of 0.7 ml are taken at 24, 48, and 72 hours incubation. Malonic acid concentration in the samples are determined by high performance liquid chromatography (with refractive index detector).

TABLE 5-1

| Strain no | MSADh Amino Acid SEQ ID | Malonic acid (g/L) | stdev (g/L) |
|---|---|---|---|
| Strain 2.11 | 45 | 6.6 | 0.078 |
| Strain 2.12 | 29 | 5.4 | 0.003 |

This study simulated a fed batch fermentation protocol where maltodextrin was continually hydrolyzed to supply a consistent feed of glucose. The study showed that the malonate-semialdehyde dehydrogenase enzymes produced in the yeast host cells were capable of generating viable malonic acid concentrations. The study further showed that substitution of a phenylalanine to a tryptophan at position 157 of SEQ ID NO: 29 in the malonate-semialdehyde dehydrogenases results in sufficient in vivo activity to produce malonic acid. Additionally, the study showed that further changing an isoleucine to a threonine at position 289 of SEQ ID NO: 45 either alone or in conjunction with a phenylalanine to a tryptophan at position 157 of SEQ ID NO: 45 was capable of producing even more malonic acid. These results also show further examples of enzymes that can be used to produce viable amounts of malonic acid.

### Example 6: Malonic Acid Production From a Fermentation with a Controlled Release of Glucose Substrate from Maltodextrin

Strains 2.9, 2.10 and 2.13 are characterized in shake flasks according to Example 5 with the following changes: The seed shake flask and the production shake flask are incubated at 34° C.

TABLE 6-1

| strain no | MSADh Amino Acid SEQ ID | malonic acid (g/L) | stdev (g/L) |
|---|---|---|---|
| Strain 2.13 | 11 | 0.3 | 0.032 |
| Strain 2.9 | 27 | 5.6 | 0.023 |
| Strain 2.10 | 27 | 11.4 | 0.052 |

This study simulated a fed batch fermentation protocol where maltodextrin was continually hydrolyzed to supply a consistent feed of glucose. This study showed that SEQ ID NO: 27 in a Pichiakudriavzevii host was capable of producing viable amounts of malonic acid. Moreover, the results showed again that changing the phenylalanine at residue position 160 to a tryptophan can improve the performance of the enzyme. The results also showed that increasing the concentration of the enzyme resulted in an increase in malonic acid concentration to exceed 10 g/L. Specifically, from the study it could be determined that increasing the copy number of malonate-semialdehyde dehydrogenase gene could increase the production of malonic acid or malonate. For example a copy number of the genes can be 2X, 3X, 4X, 5X, 6X, 7X, 8X, 9X, 10X, or higher. It is suspected that further increases in enzyme copy will follow a monotonic relationship with malonic acid production, but the examples are not so bound.

### Example 7: Malonic Acid Production From a Fermentation with a Controlled Release of Glucose Substrate from Maltodextrin in BatchFermenters.

Fermentation of Strain 2.10 is carried out in a Sartorius Ambr250 automated bioreactor system. The working volume is 200 mL. The inoculum is comprised of a two stage shake flask seed. The first stage seed is comprised of 250 mL baffled Erlenmeyer shake flasks containing 25 mL sterile seed media (composition listed in Table 7-1). A slurry made by dispensing a loop full of solid culture from a YPD plate streaked with Strain 2.10 and incubated at room temperature for 3 days, into 5 mL of sterile seed media is used to inoculate the shake flasks. The flasks are incubated at 30° C. at 300 RPM and 70% humidity in an Infors Multitron shaking incubator with a 2.5 cm throw (model AJ125C) for 16-20 hours. The second stage seed is comprised of 250 mL baffled Erlenmeyer shake flasks containing 25 mL sterile seed media and inoculated with culture from the first stage seed to an initial optical density (600 nm) of 1. The flasks are incubated at 34° C. at 300 RPM and 70% humidity in an Infers Multitron shaking incubator with a 2.5 cm throw (model AJ125C) for 4 hours. The culture from the second stage shake flask is harvested when the optical density (600 nm) of the biomass is in the range of 4-8. The harvested culture is used to inoculate the Ambr250 bioreactors at an initial optical density (600 nm) of 0.06-0.07. Optical density is measured at wavelength 600 nm with a 1 cm pathlength using a model Genesys20 Spectrophotometer (Thermo Scientific, model 4001/4). Dry cell mass is calculated from the measured OD600 value using an experimentally derived conversion factor of 1.77 OD600 units per 1 g/L, dry cell mass.

The fermentation media composition is listed in Table 7-2. The fermentation process is run at a temperature of 34° C. The fermenters are sparged with air at a flow rate of 33.3 standard mL/min and agitation is set to 1650 rpm. pH is controlled at 4.13 using 300 g/L KOH base. The process is run in a simultaneous saccharification and fermentation mode with maltodextrin as the carbon source and aminoglycosidase from Aspergillusniger (Sigma A7095) as the saccharification enzyme. 0.025 µL of aminoglycosidase per liter media is added immediately prior to inoculation. The fermentation is operated such that after a desired cell density is attained, dissolved oxygen limitation is achieved, and subsequently maintained (i.e. Dissolved oxygen (DO) < 2 %) throughout the rest of the fermentation. The onset of dissolved oxygen limitation marks the beginning of the production phase. The total fermentation time is roughly 93 hours, while the production time is 72 h. The malonic acid production metrics during this fermentation are presented in Table 7-3.

TABLE 7-1

Seed media composition

| | |
|---|---|
| Chemical | Concentration |
| glucose | 50.0 g/L |
| Glycerol | 0.375 g/L |
| MES | 13.7 g/L |
| 25x DMu salts: Table 7-1a. | 40 mL/L, |
| 1000x DM1 Full Vitamin Solution: Table 7-1b | 1 mL/L. |
| 1000x DM1 TE Solution: Table 7-1C | 1 mL/L. |

TABLE 7-1a

25X DMu salts

| | |
|---|---|
| Chemical | g/L, @ 25X |
| Urea | 57.0 |
| KH₂PO₄ | 75.0 |
| MgSO₄*7H₂O | 12.5 |
| Deionized water | Volume to 1L |

TABLE 7-1b

1000X DM1 Full Vitamin solution

| Chemical | g/L |
|---|---|
| Biotin (D-) | 0.05 |
| Ca D(+) pantothenate | 1.00 |
| Nicotinic acid | 5.00 |
| Myo-inositol | 25.00 |
| Thiamine hydrochloride | 1.00 |
| Pyridoxine hydrochloride | 1.00 |
| p-aminobenzoic acid | 0.20 |

TABLE 7-1c

1000XDM1 TE solution

| Chemical | g/L |
|---|---|
| C₁₀H₁₄N₂Na₂O₈.2H₂O | 15.00 |
| ZnSO₄.7H₂O | 4.50 |
| MnCl₂.2H₂O | 1.24 |
| CoCl₂.6H₂O | 0.30 |
| CuSO₄.5H₂O | 0.30 |
| Na₂MoO₄.2H₂O | 0.40 |
| CaCl₂.2H₂O | 4.50 |
| FeSO₄.7H₂O | 3.00 |
| H₃BO₃ | 1.00 |
| KI | 0.10 |

TABLE 7-2

Fermentation media composition

| Chemical | Concentration (g/kg) |
|---|---|
| Maltodextrin | 100 |
| Glycerol | 0.1 |
| Lubrizol(Dystar, BCC627) Antifoam (1:100 dilution) | 0.2 |
| Bulk salts aqueous solution (98 g/kg NH₄OH, 113 g/kg KOH, 310 g/kgH₃PO₄) | 3.5 |
| Urea (49.4 % solution) | 2.9 |
| MgSO₄.7H₂O | 0.25 |
| 1000X DM1 Full Vitamin solution | 1 |
| 1000X DM1 TE solution | 1 |

The Ambr250 system records real time measurements of temperature, airflow, agitation, pH, dissolved oxygen, and offgas composition. Oxygen uptake rate (OUR) and carbon dioxide exchange rate (CER) are calculated based on offgas analysis.

Samples are obtained at several time points during the course of the fermentation. These samples are used for optical density (600 nm) measurement and analyzed using high performance liquid chromatography (HPLC).

TABLE 7-3

Fermentation metrics for malonic acid production using Strain 2.10 in Ambr250 bioreactor

| Strain | MSADh Amino Acid SEQ ID | Average production phase OUR (mmol L⁻¹ h⁻¹) | Malonic acid titer at End of Fermentation (g/L) |
|---|---|---|---|
| Strain 2.10 | 27 | 17.4 | 10.2 |

This study simulated a fed batch fermentation protocol where maltodextrin was continually hydrolyzed to supply a consistent feed of glucose. This study showed that SEQ ID NO: 27 having a substitution at residue position 160 from a phenylalanine to a tryptophan in a Pichiakudriavzevii host microorganism was capable of producing viable amounts of malonic acid. Moreover, the results showed that a malonic acid concentration exceeding 10 g/L could be achieved where a medium in the fermenter was kept at a pH of 4.13. This pH is superior to most bacterial organic acid fermentations where the pH is typically maintained above 5 or even above 6.

The terms and expressions that have been employed are used as terms of description and not of limitation, and there is no intention in the use of such terms and expressions of excluding any equivalents of the features shown and described or portions thereof, but it is recognized that various modifications are possible within the scope of the embodiments of the present disclosure. Thus, it should be understood that although the present disclosure has been specifically disclosed by specific embodiments and optional features, modification and variation of the concepts herein disclosed may be resorted to by those of ordinary skill in the art, and that such modifications and variations are considered to be within the scope of embodiments of the present disclosure.

### Additional Embodiments.

The following exemplary embodiments are provided, the numbering of which is not to be construed as designating levels of importance:
Embodiment 1 provides an engineered microorganism capable of producing malonic acid, malonate, esters of malonic acid, or mixtures thereof, the engineered microorganism comprising: a heterologous malonate-semialdehyde dehydrogenase that comprises at least 90% sequence identity to any one of SEQ ID Nos: 7, 9, 11, 13, 15, 17, 19, 21, 23, 27, 29, and 31, wherein the engineered microorganism is capable of producing about 9 g/L to about 250 g/L of malonic acid, malonate, esters of malonic acid, or mixtures thereof at a pH between 2 and 7.0.
Embodiment 2 provides the engineered microorganism according to Embodiment 1, wherein the malonate-semialdehyde dehydrogenase comprises at least 95% sequence identity to any one of SEQ ID Nos: 7, 9, 11, 13, 15, 17, 19, 21, 23, 27, 29, and 31.
Embodiment 3 provides the engineered microorganism of any one of Embodiments 1 or 2, wherein the malonate-semialdehyde dehydrogenase catalyzes the conversion of a malonate-semi aldehyde to malonic acid, malonate, esters of malonic acid, or mixtures thereof.
Embodiment 4 provides the engineered microorganism of any one of Embodiments 1-3, comprising at least 90% sequence identity to SEQ ID No: 7.
Embodiment 5 provides the engineered microorganism of any one of Embodiments 1-4, comprising at least 95% sequence identity to SEQ ID No: 7.
Embodiment 6 provides the engineered microorganism of any one of Embodiments 4 or 5, comprising at least 90% sequence identity to any one of SEQ ID Nos: 11 or 27
Embodiment 7 provides the engineered microorganism of any one of Embodiments 4-6, comprising at least 95% sequence identity to any one of SEQ ID Nos: 11 or 27.
Embodiment 8 provides the engineered microorganism of any one of Embodiments 1-7, wherein an amino acid residue of a polypepti de that aligns with amino acid residue 160 of SEQ ID NO: 11 is not phenylalanine.
Embodiment 9 provides the engineered microorganism of any one of Embodiments 1-8, wherein an amino acid residue of a polypeptide that aligns with amino acid residue 160 of SEQ ID NO: 11 is tryptophan.
Embodiment 10 provides the engineered microorganism of any one of Embodiments 1-9, wherein the engineered microorganism is capable of producing about 10 g/L to about 200 g/L of the malonic acid, malonate, esters of malonic acid, or mixtures thereof.
Embodiment 11 provides the engineered microorganism of any one of Embodiments 1-10, wherein the engineered microorganism is capable of producing about 50 g/L to about 150 g/L of the malonic acid, malonate, esters of malonic acid, or mixtures thereof.
Embodiment 12 provides the engineered microorganism of any one of Embodiments 1-11, wherein the engineered microorganism further comprises:
   a polypeptide capable of converting phosphoenolpyruvate (PEP) to oxaloacetate (OAA);
   a polypeptide capable of converting pyruvate to oxaloacetate (OAA);
   a polypeptide capable of converting oxaloacetate (OAA) to aspartate;
   a polypeptide capable of converting aspartate to beta alanine;
   a polypeptide capable of converting a beta alanine to malonate-semialdehyde, or
   a mixture thereof.
Embodiment 13 provides the engineered microorganism of any one of Embodiments 1-12, wherein the engineered microorganism further comprises:
   at least one of a polypeptide capable of converting phosphoenolpyruvate (PEP) to oxaloacetate (OAA) and a polypeptide capable of converting pyruvate to oxaloacetate (OAA);
   a polypeptide capable of converting oxaloacetate (OAA) to aspartate;
   a polypeptide capable of converting aspartate to beta alanine; and
   a polypeptide capable of converting a beta alanine to malonate-semialdehyde.
Embodiment 14 provides the engineered microorganism of Embodiment 13, wherein the polypeptide capable of converting phosphoenolpyruvate (PEP) to oxaloacetate (OAA) comprises a phosphoenolpyruvate carboxytransphosphorylase or phosphoenolpyruvate carboxylase.
Embodiment 15 provides the engineered microorganism of any one of Embodiments 13 or 14, wherein the polypeptide capable of converting pyruvate to oxaloacetate (OAA) comprises a pyruvate carboxylase.
Embodiment 16 provides the engineered microorganism of any one of Embodiments 13-15, wherein the polypeptide capable of converting oxaloacetate (OAA) to aspartate comprises an aspartate aminotransferase (AAT).
Embodiment 17 provides the engineered microorganism of any one of Embodiments 13-16, wherein the polypeptide capable of converting beta alanine to malonate-semialdehyde comprises a beta-alanine aminotransferase or a β-alanine-pyruvate aminotransferase.
Embodiment 18 provides the engineered microorganism of any one of Embodiments 1-17, wherein the engineered microorganism has reduced pyruvate decarboxylase (PDC) activity compared to a native form of the engineered microorganism.
Embodiment 19 provides the engineered microorganism of any one of Embodiments 1-18, wherein the engineered microorganism has reduced GPD activity compared to a native form of the engineered microorganism.
Embodiment 20 provides the engineered microorganism of any one of Embodiment 1-19, wherein the engineered microorganism further comprises an exogenous gene encoding a polypeptide capable of converting aspartate to beta-alanine.
Embodiment 21 provides the engineered microorganism of Embodiment 20, wherein the polypeptide capable of converting aspartate to beta-alanine is panD) or aspartate decarboxylase (ADC) and is optionally heterologous.
Embodiment 22 provides the engineered microorganism of any one of Embodiments 1-21, wherein the engineered microorganism has reduced malonyl-CoA reductase, 3-HPDH, HIBADH, 4-hydroxybutyrate dehydrogenase, or 3-HP dehydrogenase activity as compared to a native form of the engineered microorganism.
Embodiment 23 provides the engineered microorganism of any one of Embodiments 1-22, wherein the engineered microorganism comprises a fungus.
Embodiment 24 provides the engineered microorganism of Embodiment 23, wherein the fungus comprises a yeast.
Embodiment 25 provides the engineered microorganism of any one of Embodiments 1-24, wherein the microorganism comprises Saccharomycescerevisiae, Kluyveromyceslactis, Kluyveromyces marxianus, Yarrowialipolytica, Pichiakudriavzevii, Schizosaccharomycespombe, or a mixture thereof.
Embodiment 26 provides the engineered microorganism of any one of Embodiments 1-25, wherein the engineered microorganism comprises a bacteria.
Embodiment 27 provides the engineered microorganism of any one of Embodiments 1-26, wherein the engineered microorganism comprises Streptococcus, Lactobacillus, Bacillus, Escherichia, Salmonella, Neisseria, Acetobactor, Arthrobacter, Aspergillus, Bifdobacterium, Corynebacterium, Pseudomonas, or a mixture thereof.
Embodiment 28 provides the engineered microorganism of any one of Embodiments 1-27, wherein the engineered microorganism comprises Escherichiacoli or Pichiakudriavzevii.
Embodiment 29 provides the engineered microorganism of any one of Embodiments 1-28, wherein the engineered microorganism is capable of growing at a pH of less than about 6 in the presence of malonic acid, malonate, esters of malonic acid, or mixtures thereof at a concentration of about 20 g/L.
Embodiment 30 provides the engineered microorganism of any one of Embodiments 1-29, wherein the engineered microorganism is capable of growing at a pH of less than about 4 in the presence of malonic acid, malonate, esters of malonic acid, or mixtures thereof at a concentration of about 20 g/L.
Embodiment 31 provides an engineered microorganism capable of producing malonate, the engineered microorganism comprising: a heterologous gene, which encodes the malonate-semialdehyde dehydrogenase of any one of Embodiments 1-30.
Embodiment 32 provides the engineered microorganism of any one of Embodiments 1-31, wherein the engineered microorganism further comprises:
   an exogenous gene encoding a polypeptide capable of converting phosphoenolpyruvate (PEP) to oxaloacetate (OAA);
   an exogenous gene encoding a polypeptide capable of converting pyruvate to oxaloacetate (OAA);
   an exogenous gene encoding a polypeptide capable of converting oxaloacetate (OAA) to aspartate;
   an exogenous gene encoding a polypeptide capable of converting aspartate to beta alanine;
   an exogenous gene encoding a polypeptide capable of converting a beta alanine to malonate-semialdehyde, or
   a mixture thereof.
Embodiment 33 provides the engineered microorganism of any one of Embodiments 1-32, wherein the engineered microorganism is capable of producing about 9 g/L to about 250 g/L of malonic acid, malonate, esters of malonic acid, or mixtures thereof at a pH between 2.5 and 4.0.
Embodiment 34 provides the engineered microorganism of any one of Embodiments 1-32, wherein the engineered microorganism is capable of producing about 9 g/L to about 250 g/L of malonic acid, malonate, esters of malonic acid, or mixtures thereof at a pH between 3.5 and 6.0.
Embodiment 35 provides the engineered microorganism of any one of Embodiments 1-34, wherein the engineered microorganism further comprises:
   at least one of an exogenous gene encoding a polypeptide capable of converting phosphoenolpyruvate (PEP) to oxaloacetate (OAA) and an exogenous gene encoding a polypeptide capable of converting pyruvate to oxaloacetate
   (OAA); an exogenous gene encoding a polypeptide capable of converting oxaloacetate (OAA) to aspartate;
   an exogenous gene encoding a polypeptide capable of converting aspartate to beta alanine; and
   an exogenous gene encoding a polypeptide capable of converting a beta alanine to malonate-semialdehyde.
Embodiment 36 provides an engineered microorganism capable of producing malonic acid, malonate, esters of malonic acid, or mixtures thereof, the engineered microorganism comprising: a heterologous malonate-semialdehyde dehydrogenase comprising at least 90% sequence identity to SEQ ID No: 11, wherein the amino acid residue of the polypeptide that aligns with amino acid residue 160 of SEQ ID NO: 11 is not phenylalanine and the engineered microorganism is capable of producing about 9 g/L to about 250 g/L of malonic acid, malonate, esters of malonic acid, or mixtures thereof.
Embodiment 37 provides an engineered microorganism capable of producing malonic acid, malonate, esters of malonic acid, or mixtures thereof, the engineered microorganism comprising: a heterologous malonate-semialdehyde dehydrogenase comprising at least 90% sequence identity to SEQ ID No: 11 , wherein the amino acid residue of the polypeptide that aligns with amino acid residue 160 of SEQ ID NO: 11 is tryptophan and the engineered microorganism is capable of producing about 9 g/L to about 250 g/L of malonic acid, malonate, esters of malonic acid, or mixtures thereof.
Embodiment 38 provides a fermentation method for producing malonic acid, malonate, esters of malonic acid, or mixtures thereof, the method comprising:
   culturing an engineered microorganism comprising a heterologous malonate-semialdehyde dehydrogenase that comprises at least 90% sequence identity to any one of SEQ ID Nos: 7, 9, 11, 13, 15, 17, 19, 21, 23, 27, 29, and 31 in the presence of a medium comprising at least one carbon source; and
   producing about 9 g/L to about 250 g/L of malonic acid, malonate, esters of malonic acid, or mixtures thereof.
Embodiment 39 provides the fermentation method of Embodiment 38, further comprising isolating the malonic acid, malonate, esters of malonic acid, or mixtures thereof.
Embodiment 40 provides the fermentation method of any one of Embodiments 38 or 39, wherein the at least one carbon source is selected from glucose, xylose, arabinose, sucrose, fructose, cellulose, glucose oligomers, and glycerol.
Embodiment 41 provides the fermentation method of any one of Embodiments of 38-40, wherein the at least one carbon source comprises glucose.
Embodiment 42 provides the fermentation method of any one of Embodiments 38-41, wherein the medium at the end of fermentation is at a pH of less than 6.
Embodiment 43 provides the fermentation method of any one of Embodiments 38-42, wherein the medium at the end of fermentation is at a pH in a range of from about 3 to about 4.5.
Embodiment 44 provides the fermentation method of any one of Embodiments 38-43, wherein the fermentation method comprises a batch or fed batch method.
Embodiment 45 provides the fermentation method of Embodiment 44, wherein the fermentation method comprises simultaneous saccharification and fermentation.
Embodiment 46 provides the fermentation method of any one of Embodiments 38-45, wherein the method is carried out in aerobic, microaerobic or anaerobic conditions.
Embodiment 47 provides the fermentation method of Embodiment 46, wherein an oxygen transfer rate is between 10 mmol 1⁻¹ h⁻¹ and 60 mmol 1⁻¹ h⁻¹.
Embodiment 48 provides the fermentation method of any one of Embodiments 46 or 47, wherein an oxygen transfer rate is between 25 mmol 1⁻¹ h⁻¹ and 45 mmol 1⁻¹ h⁻¹.
Embodiment 49 provides the fermentation method of any one of Embodiments 38-48, wherein less than about 5 g/L of ethanol is produced after about 36 hours.
Embodiment 50 provides the fermentation method of any one of Embodiments 38-49, wherein the engineered microorganism further comprises malonate-semialdehyde.
Embodiment 51 provides the fermentation method of any one of Embodiments 38-50, wherein the malonate-semialdehyde dehydrogenase comprises at least 95% sequence identity to any one of SEQ ID Nos: 7, 9, 11, 13, 15, 17, 19, 21, 23, 27, 29, and 31.
Embodiment 52 provides the fermentation method of any one of Embodiments 38-51, wherein the malonate-semialdehyde dehydrogenase comprises at least 90% sequence identity to SEQ ID No: 7.
Embodiment 53 provides the fermentation method of any one of Embodiments 38-52, wherein the malonate-semialdehyde dehydrogenase comprises at least 95% sequence identity to SEQ ID No: 7.
Embodiment 54 provides the fermentation method of any one of Embodiments 38-53, wherein the malonate-semialdehyde dehydrogenase comprises at least 90% sequence identity to any one of SEQ ID Nos: 11 or 27.
Embodiment 55 provides the fermentation method of any one of Embodiments 38-54, wherein the malonate-semialdehyde dehydrogenase comprises at least 95% sequence identity to any one of SEQ ID Nos: 11 or 27.
Embodiment 56 provides the fermentation method of any one of Embodiments 38-55, wherein an amino acid residue of a polypeptide that aligns with amino acid residue 160 of SEQ ID NO: 11 is not phenylalanine.
Embodiment 57 provides the fermentation method of any one of Embodiments 38-56, wherein an amino acid residue of a polypeptide that aligns with amino acid residue 160 of SEQ ID NO: 11 is tryptophan.
Embodiment 58 provides the fermentation method of any one of Embodiments 38-57, wherein the engineered microorganism is capable of producing about 10 g/L to about 200 g/L of the malonic acid, malonate, esters of malonic acid, or mixtures thereof.
Embodiment 59 provides the fermentation method of any one of Embodiments 38-58, wherein the engineered microorganism is capable of producing about 50 g/L to about 150 g/L of the malonic acid, malonate, esters of malonic acid, or mixtures thereof.
Embodiment 60 provides the fermentation method of any one of Embodiments 38-59, wherein the engineered microorganism further comprises:
   a polypeptide capable of converting phosphoenolpyruvate (PEP) to oxaloacetate (OAA);
   a polypeptide capable of converting pyruvate to oxaloacetate (OAA);
   a polypeptide capable of converting oxaloacetate (OAA) to aspartate;
   a polypeptide capable of converting aspartate to beta alanine;
   a polypeptide capable of converting a beta alanine to malonate-semialdehyde, or
   a mixture thereof.
Embodiment 61 provides the fermentation method of any one of Embodiments 38-60, wherein the engineered microorganism further comprises:
   at least one of a polypeptide capable of converting phosphoenolpyruvate (PEP) to oxaloacetate (OAA) and a polypeptide capable of converting pyruvate to oxaloacetate (OAA);
   a polypeptide capable of converting oxaloacetate (OAA) to aspartate;
   a polypeptide capable of converting aspartate to beta alanine; and
   a polypeptide capable of converting a beta alanine to malonate-semialdehyde.
Embodiment 62 provides the fermentation method of any one of Embodiments 60 or 61, wherein the polypeptide capable of converting phosphoenolpyruvate (PEP) to oxaloacetate (OAA) comprises a phosphoenolpyruvate carboxytransphosphorylase or phosphoenolpyruvate carboxylase.
Embodiment 63 provides the fermentation method of any one of Embodiments 60-62, wherein the polypeptide capable of converting pyruvate to oxaloacetate (OAA) comprises a pyruvate carboxylase.
Embodiment 64 provides the fermentation method of any one of Embodiments 60-63, wherein the polypeptide capable of converting oxaloacetate (OAA) to aspartate comprises an aspartate aminotransferase (AAT).
Embodiment 65 provides the fermentation method of any one of Embodiments 60-64, wherein the polypeptide capable of converting beta alanine to malonate-semialdehyde comprises a beta-alanine aminotransferase or a β-alanine-pyruvate aminotransferase.
Embodiment 66 provides the fermentation method of any one of Embodiments 38-65, wherein the engineered microorganism has reduced pyruvate decarboxylase (PDC) activity compared to a native form of the engineered microorganism.
Embodiment 67 provides the fermentation method of any one of Embodiments 38-66, wherein the engineered microorganism has reduced GPD activity compared to a native form of the engineered microorganism.
Embodiment 68 provides the fermentation method of any one of Embodiments 38-67, wherein the engineered microorganism further comprises an exogenous gene encoding a polypeptide capable of converting aspartate to beta-alanine.
Embodiment 69 provides the fermentation method of Embodiment 68, wherein the polypeptide capable of converting aspartate to beta-alanine is panD or aspartate decarboxylase (ADC) and is optionally heterologous.
Embodiment 70 provides the fermentation method of any one of Embodiments 38-69, wherein the engineered microorganism has reduced malonyl-CoA reductase, 3-HPDH, HIBADH, 4-hydroxybutyrate dehydrogenase, or 3-HP dehydrogenase activity as compared to a native form of the engineered microorganism.
Embodiment 71 provides the fermentation method of any one of Embodiments 38-70, wherein the engineered microorganism comprises a fungus.
Embodiment 72 provides the fermentation method of Embodiment 71, wherein the fungus comprises a yeast.
Embodiment 73 provides the fermentation method of any one of Embodiments 38-72, wherein the microorganism comprises Saccharomycescerevisiae, Kluyveromyceslactis, Kluyveromyces marxiamus, Yarrowialipolytica, Pichiakudriavzevii, Schizosaccharomycespombe, or a mixture thereof.
Embodiment 74 provides the fermentation method of any one of Embodiments 38-73, wherein the engineered microorganism comprises a bacteria.
Embodiment 75 provides the fermentation method of any one of Embodiments 38-74, wherein the engineered microorganism comprises Streptococcus, Lactobacillus, Bacillus, Escherichia, Salmonella, Neisseria, Acetobactor, Arthrobacter, Aspergillus, Bifdobacterium, Corynebacterium, Pseudomanas, or a mixture thereof.
Embodiment 76 provides the fermentation method of any one of Embodiments 38-75, wherein the engineered microorganism comprises Escherichiacoli or Pichiakudriavzevii.
Embodiment 77 provides the fermentation method of any one of Embodiments 38-76, wherein the engineered microorganism is capable of growing at a pH of less than about 6 in the presence of malonic acid, malonate, esters of malonic acid, or mixtures thereof at a concentration of about 20 g/L.
Embodiment 78 provides the fermentation method of any one of Embodiments 38-77, wherein the engineered microorganism is capable of growing at a pH of less than about 4 in the presence of malonic acid, malonate, esters of malonic acid, or mixtures thereof at a concentration of about 20 g/L.
Embodiment 79 provides the fermentation method of any one of Embodiments 38-78, wherein the engineered microorganism comprises: a heterologous gene that is not present in the native form of the engineered microorganism, which encodes the malonate-semialdehyde dehydrogenase of any one of Embodiments 36-30.
Embodiment 80 provides the fermentation method of any one of Embodiments 38-79, wherein the engineered microorganism comprises:
   an exogenous gene encoding a polypeptide capable of converting phosphoenolpyruvate (PEP) to oxaloacetate (OAA);
   an exogenous gene encoding a polypeptide capable of converting pyruvate to oxaloacetate (OAA);
   an exogenous gene encoding a polypeptide capable of converting oxaloacetate (OAA) to aspartate;
   an exogenous gene encoding a polypeptide capable of converting aspartate to beta alanine;
   an exogenous gene encoding a polypeptide capable of converting a beta alanine to malonate-semialdehyde, or
   a mixture thereof.
Embodiment 81 provides the fermentation method of any one of Embodiments 38-80, wherein the engineered microorganism comprises:
   at least one of an exogenous gene encoding a polypeptide capable of converting phosphoenolpyruvate (PEP) to oxaloacetate (OAA) and an exogenous gene encoding a polypeptide capable of converting pyruvate to oxaloacetate (OAA);
   an exogenous gene encoding a polypeptide capable of converting oxaloacetate (OAA) to aspartate;
   an exogenous gene encoding a polypeptide capable of converting aspartate to beta alanine; and
   an exogenous gene encoding a polypeptide capable of converting a beta alanine to malonate-semialdehyde.
Embodiment 82 provides a malonate-semialdehyde dehydrogenase formed according to the method of any one of Embodiments 38-81.
Embodiment 83 provides a malonate-semialdehyde dehydrogenase comprising at least 90% sequence identity to any one of SEQ ID Nos: 7, 9, 11, 13, 15, 17, 19, 21, 23, 27, 29, and 31 formed according to the method of any one of Embodiments 38-82.
Embodiment 84 provides a heterologous malonate-semialdehyde dehydrogenase comprising at least 95% sequence identity to any one of SEQ ID Nos: 7, 9, 11, 13, 15, 17, 19, 21, 23, 27, 29, and 31 formed according to the method of any one of Embodiments 38-83.
Embodiment 85 provides the engineered microorganism of any one of Embodiments 1-84, wherein the engineered microorganism is capable of producing malonic acid.
Embodiment 86 provides the engineered microorganism of any one of Embodiments 1-85, wherein the engineered microorganism is capable of producing malonate.
Embodiment 87 provides the engineered microorganism of any one of Embodiments 1-86, wherein the engineered microorganism comprises a bacteria, for example Escherichiacoli.
Embodiment 88 provides the engineered microorganism of any one of Embodiments 1-86, wherein the engineered microorganism comprises Pichiakudriavzevii.

1. An engineered microorganism capable of producing malonic acid, malonate, esters of malonic acid, or mixtures thereof, the engineered microorganism comprising:
a heterologous malonate-semialdehyde dehydrogenase that comprises at least 90% sequence identity to any one of SEQ ID Nos: 7, 9, 11, 13, 15, 17, 19, 21, 23, 27, 29, and 31, wherein the engineered microorganism is capable of producing about 9 g/L to about 250 g/L of malonic acid, malonate, esters of malonic acid, or mixtures thereof at a pH between 2 and 7.0.

2. The engineered microorganism according to claim 1, wherein the malonate-semialdehyde dehydrogenase comprises at least 95% sequence identity to any one of SEQ ID Nos: 7, 9, 11, 13, 15, 17, 19, 21, 23, 27, 29, and 31.

3. The engineered microorganism of claim 2, wherein the malonate-semialdehyde dehydrogenase catalyzes the conversion of a malonate-semialdehyde to malonic acid, malonate, esters of malonic acid, or mixtures thereof.

4. (canceled)

5. (canceled)

6. The engineered microorganism of claim 1, comprising at least 90% sequence identity to any one of SEQ ID Nos: 11 or 27.

7. (canceled)

8. The engineered microorganism of claim 1, wherein an amino acid residue of a polypeptide that aligns with amino acid residue 160 of SEQ ID NO: 11 is not phenylalanine.

9. The engineered microorganism of claim 1, wherein an amino acid residue of a polypeptide that aligns with amino acid residue 160 of SEQ ID NO: 11 is tryptophan.

10. (canceled)

11. (canceled)

12. The engineered microorganism of claim 1, wherein the engineered microorganism further comprises:
a polypeptide capable of converting phosphoenolpyruvate (PEP) to oxaloacetate (OAA);
a polypeptide capable of converting pyruvate to oxaloacetate (OAA);
a polypeptide capable of converting oxaloacetate (OAA) to aspartate;
a polypeptide capable of converting aspartate to beta alanine;
a polypeptide capable of converting a beta alanine to malonate-semialdehyde, or
a mixture thereof.

13-17. (canceled)

18. The engineered microorganism of claim 1, wherein the engineered microorganism has reduced pyruvate decarboxylase (PDC) activity compared to a native form of the engineered microorganism.

19. The engineered microorganism of claim 18, wherein the engineered microorganism has reduced GPD activity compared to a native form of the engineered microorganism.

20. The engineered microorganism of claim 1, wherein the engineered microorganism further comprises an exogenous gene encoding a polypeptide capable of converting aspartate to beta-alanine.

21. The engineered microorganism of claim 20, wherein the polypeptide capable of converting aspartate to beta-alanine is panD or aspartate decarboxylase (ADC) and is optionally heterologous.

22. (canceled)

23. The engineered microorganism of claims 1, wherein the engineered microorganism comprises a fungus.

24. The engineered microorganism of claim 23, wherein the fungus comprises a yeast.

25. The engineered microorganism of claim 1, wherein the microorganism comprises Saccharomycescerevisiae, Kluyveromyceslactis, Kluyveromyces marxianus, Yarrowialipolytica, Pichiakudriavzevii, Schizosaccharomycespombe, or a mixture thereof.

26. (canceled)

27. (canceled)

28. The engineered microorganism of claim 1, wherein the engineered microorganism comprises Pichiakudriavzevii.

29. (canceled)

30. The engineered microorganism of claim 1, wherein the engineered microorganism is capable of growing at a pH of less than about 4 in the presence of malonic acid, malonate, esters of malonic acid, or mixtures thereof at a concentration of about 20 g/L.

31. (canceled)

32. (canceled)

33. The engineered microorganism of claim 1, wherein the engineered microorganism is capable of producing about 9 g/L to about 250 g/L of malonic acid, malonate, esters of malonic acid, or mixtures thereof at a pH between 2.5 and 4.0.

34. The engineered microorganism of claim 1, wherein the engineered microorganism is capable of producing about 9 g/L to about 250 g/L of malonic acid, malonate, esters of malonic acid, or mixtures thereof at a pH between 3.5 and 6.0.

35. (canceled)

36. An engineered microorganism capable of producing malonic acid, malonate, esters of malonic acid, or mixtures thereof, the engineered microorganism comprising:
a heterologous malonate-semialdehyde dehydrogenase comprising at least 90% sequence identity to SEQ ID No: 11, wherein the amino acid residue of the polypeptide that aligns with amino acid residue 160 of SEQ ID NO: 11 is not phenylalanine and the engineered microorganism is capable of producing about 9 g/L to about 250 g/L of malonic acid, malonate, esters of malonic acid, or mixtures thereof.

37. An engineered microorganism capable of producing malonic acid, malonate, esters of malonic acid, or mixtures thereof, the engineered microorganism comprising:
a heterologous malonate-semialdehyde dehydrogenase comprising at least 90% sequence identity to SEQ ID No: 11 , wherein the amino acid residue of the polypeptide that aligns with amino acid residue 160 of SEQ ID NO: 11 is tryptophan and the engineered microorganism is capable of producing about 9 g/L to about 250 g/L of malonic acid, malonate, esters of malonic acid, or mixtures thereof.

38-49. (canceled)
